(19) United States
(12) Patent Application Publication
(10) Pub. No.: US 2008/0254318 A1
(43) Pub. Date: Oct. 16, 2008

(54) Carbazole Derivative, and Light-Emitting Element and Light-Emitting Device Using the Carbzole Derivative

(75) Inventors: Harue Nakashima, Kanagawa (JP); Daisuke Kumaki, Nigata (JP); Kumi Kojima, Tokyo (JP); Satoshi Seo, Kanagawa (JP); Sachiko Kawakami, Kanagawa (JP)

Correspondence Address: ERIC ROBINSON, PMB 955, 21010 SOUTHBANK ST., POTOMAC FALLS, VA, 20165, US

(73) Assignee: Semiconductor Energy Laboratory Co., Ltd., Atsugi-shi (JP)

(21) Appl. No.: 10/585,326

(22) PCT Filed: Dec. 26, 2005
(86) PCT No.: PCT/JP2005/024212

§ 371 Date: Jul. 6, 2006

(30) Foreign Application Priority Data
Dec. 28, 2004 (JP) 2004-381155
Mar. 23, 2005 (JP) 2005-085020

(51) Int. Cl. H01J 1/63; C07D 209/88; C07D 215/38

(52) U.S. Cl. 428/690; 548/440; 546/171

(57) ABSTRACT

The present invention provides a material having excellent hole injecting and hole transporting properties. Moreover, the present invention provides a light-emitting element and a light-emitting device using the material having excellent hole injecting and hole transporting properties. The present invention provides a carbazole derivative represented by the general formula (1). By applying the carbazole derivative of the present invention to a light-emitting element or a light-emitting device, a lower driving voltage, enhanced emission efficiency, a longer lifetime and enhanced reliability of the light-emitting element or the light-emitting device can be realized.

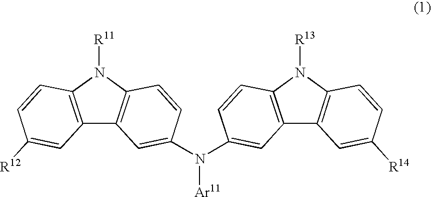

### TECHNICAL FIELD

The present invention relates to carbazole derivatives. The present invention relates to a light-emitting element having a pair of electrodes and a layer including a luminescent substance, which provides light emission when a voltage is applied thereto. Further, the present invention relates to a light-emitting device having such a light-emitting element.

### BACKGROUND ART

A light-emitting element with the use of a light-emitting material has a feature such as thin-and-light, rapid response, low DC voltage driving, and is expected to be applied for flat panel displays of the next generation. In addition, it is said that a light-emitting device in which light-emitting elements are arranged in matrix is superior to a conventional liquid crystal display device in viewing angle and visibility.

A light-emitting element is said to have the following light-emission mechanism; voltage is applied to a light-emitting layer sandwiched between a pair of electrodes, electrons injected from a cathode and holes injected from an anode are recombined in a light-emission center of the light-emitting layer to form molecular excitons, and then light is emitted by releasing energy when the molecular exciton returns to the ground state. As the excited state, a singlet-excited state and a triplet-excited state are known, and the light emission is considered possible via either one of the excited states.

Such a light-emitting element has problems about materials. In order to enhance the characteristic of such a light-emitting element, the improvement of the element structure, the development of the material, and so on have been conducted.

As an example of a material used for a layer including a luminescent substance, a material with a carbazole skeleton (a carbazole derivative) which has excellent photoconductivity, is given. Specifically, 1,3,5-[4-(N-carbazolyl)phenyl]benzene (TCBP) is given (Reference 1: Japanese Patent No. 3210481).

TCBP has been proposed as a material for forming a hole transporting layer. However, most of materials having a carbazole skeleton exhibit high ionization potential, and has a poor hole injecting property from an electrode.

On the other hand, as materials which are often used as hole injecting or hole transporting materials, for example, 4,4′-bis(N-{4-[N,N-bis(3-methyl phenyl)amino]phenyl}-N-phenylamino)biphenyl (DNTPD) is given (Reference 2: Japanese Patent Laid-Open No. H9-301934).

DNTPD can be superior in hole injecting property since its ionization potential is small. In addition, DNTPD also has a hole transporting property and is often used for a hole injecting layer or a hole transporting layer of a light-emitting element. However, since DNTPD does not exhibit favorable property, a material having more excellent property is needed.

### DISCLOSURE OF INVENTION

In view of the above problems, it is an object of the present invention to provide a material having an excellent hole injecting property and an excellent hole transporting property. Moreover, it is another object of the present invention to provide a light-emitting element and a light-emitting device using a material having an excellent hole injecting property and an excellent hole transporting property.

The present inventors have found out that a carbazole derivative represented by the general formula (1) exhibits an excellent hole injecting property and an excellent hole transporting property.

Therefore, the present invention provides a carbazole derivative represented by the general formula (1).

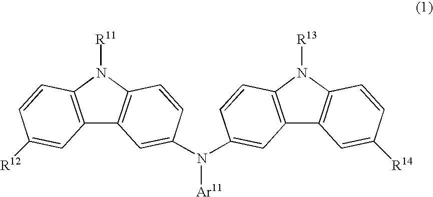

wherein R¹¹ and R¹³ are each the same or different, and represents hydrogen, an alkyl group having 1 to 6 carbon atoms, an aryl group having 6 to 25 carbon atoms, a heteroaryl group having carbon atoms 5 to 9, an arylalkyl group, or an acyl group having 1 to 7 carbon atoms; Ar¹¹ represents an aryl group having 6 to 25 carbon atoms or a heteroaryl group having carbon atoms 5 to 9; R¹² represents hydrogen, an alkyl group having carbon atoms 1 to 6, or an aryl group having carbon atoms 6 to 12; R¹⁴ represents hydrogen, an alkyl group having carbon atoms 1 to 6, an aryl group having carbon atoms 6 to 12, or a substituent shown by the general formula (2); in the substituent shown by the general formula (2), R¹⁵ represents hydrogen, an alkyl group having carbon atoms 1 to 6, an aryl group having carbon atoms 6 to 25, a heteroaryl group having carbon atoms 5 to 9, an aryl alkyl group, or an acyl group having carbon atoms 1 to 7; Ar¹² represents an aryl group having carbon atoms 6 to 25, or a heteroaryl group having carbon atoms 5 to 9; and R¹⁶ represents hydrogen, an alkyl group having carbon atoms 1 to 6, or an aryl group having carbon atoms 6 to 12.

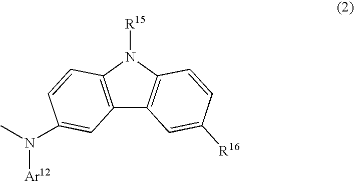

In the general formula (1), either R¹¹ or R¹³ preferably represents an aryl group having carbon atoms 6 to 25, or a heteroaryl group having carbon atoms 5 to 9. More preferably, R¹¹ and R¹³ are either an aryl group having carbon atoms 6 to 25, or a heteroaryl group having carbon atoms 5 to 9. A substituent binding with nitrogen of a carbazole skeleton employs an aryl group having carbon atoms 6 to 25, or a heteroaryl group having carbon atoms 5 to 9, and thus, an effect that a carrier transporting property is enhanced, can be obtained.

In addition, in the general formula (1), R¹² preferably represents hydrogen, tert-butyl, phenyl, or biphenyl.

In addition, in the general formula (1), R¹⁴ preferably represents hydrogen, tert-butyl, phenyl, or biphenyl.

In the general formula (1), R¹⁴ preferably represents a substituent represented by the general formula (2). A carbazole derivative having excellent heat-resistance can be obtained by using a substituent shown by the general formula (2) for R⁴. In addition, in the general formula (2), R¹⁵ preferably represents an aryl group having carbon atoms 6 to 25, or a hetero aryl group having carbon atoms 5 to 9. A substituent binding with nitrogen of a carbazole skeleton employs an aryl group having carbon atoms 6 to 25, or a heteroaryl group having carbon atoms 5 to 9, and thus, an effect that a carrier transporting property is enhanced, can be obtained. In addition, in the general formula (2), R¹⁶ preferably represents hydrogen, tert-butyl, phenyl, or biphenyl.

The present inventors have found out that a carbazole derivative represented by the general formula (3) exhibits excellent hole injecting and hole transporting properties.

Therefore, the present invention provides a carbazole derivative represented by the general formula (3).

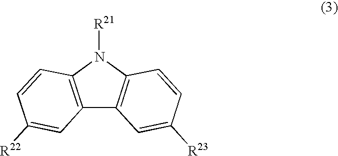

wherein R²¹ represents hydrogen, an alkyl group having 1 to 6 carbon atoms, an aryl group having 6 to 25 carbon atoms, a heteroaryl group having carbon atoms 5 to 9, an arylalkyl group, or an acyl group having 1 to 7 carbon atoms; R²² represents hydrogen, an alkyl group having carbon atoms 1 to 6, or an aryl group having carbon atoms 6 to 12; R²³ represents a substituent shown by the general formula (4); in the substituent shown by the general formula (4), R²⁴ represents hydrogen, an alkyl group having carbon atoms 1 to 6, an aryl group having carbon atoms 6 to 25, a heteroaryl group having carbon atoms 5 to 9, an aryl alkyl group, or an acyl group having carbon atoms 1 to 7; Ar²¹ represents an aryl group having carbon atoms 6 to 25, or a heteroaryl group having carbon atoms 5 to 9; and R²⁵ represents hydrogen, an alkyl group having carbon atoms 1 to 6, or an aryl group having carbon atoms 6 to 12.

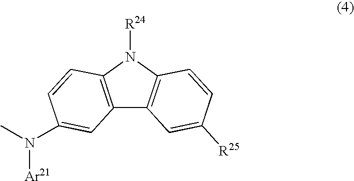

In the above structure, R²² preferably represents hydrogen, tert-butyl, phenyl, or biphenyl.

Another structure of the present invention is a carbazole derivative having a structure represented by the general formula (5).

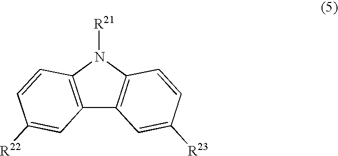

wherein R²¹ represents hydrogen, an alkyl group having carbon atoms 1 to 6, an aryl group having carbon atoms 6 to 25, a heteroaryl group having carbon atoms 5 to 9, and an arylalkyl group, or an acyl group having carbon atoms 1 to 7; R²² and R²³ represent a substituent represented by the general formula (6); in the substituent represented by the general formula (6), R²⁴ represents hydrogen, an alkyl group having carbon atoms 1 to 6, an aryl group having carbon atoms 6 to 25, a heteroaryl group having carbon atoms 5 to 9, an arylalkyl group, or an acyl group having carbon atoms 1 to 7; Ar²¹ represents an aryl group having carbon atoms 6 to 25, or a heteroaryl group having carbon atoms 5 to 9; and R²⁵ represents hydrogen, an alkyl group having carbon atoms 1 to 6, or a heteroaryl group having carbon atoms 6 to 12.

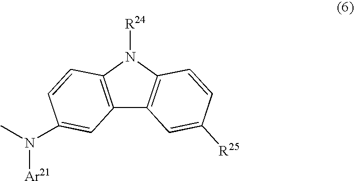

In the above structure, R²⁵ preferably represents hydrogen, tert-butyl, phenyl, or biphenyl.

In addition, in the above structure, R²⁴ preferably represents an aryl group having carbon atoms 6 to 25 or a heteroaryl group having carbon atoms 5 to 9.

In the above structure, R²¹ preferably represents an aryl group having carbon atoms 6 to 25, or a heteroaryl group having carbon atoms 5 to 9.

A substituent binding with nitrogen of a carbazole skeleton employs an aryl group having carbon atoms 6 to 25, or a heteroaryl group having carbon atoms 5 to 9, and thus, an effect that a carrier transporting property is enhanced, can be obtained.

Another structure of the present invention is a carbazole derivative having a structure shown by the general formula (7).

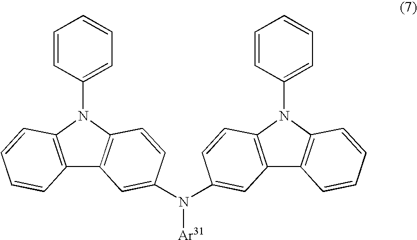

wherein Ar³¹ represents phenyl or naphthyl.

In addition, another structure of the present invention is a carbazole derivative having a structure shown by the general formula (8).

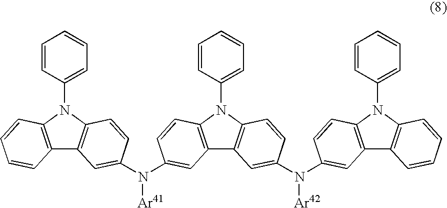

wherein Ar⁴¹ and Ar⁴² may each be the same or different, and represent phenyl or naphthyl.

In addition, a carbazole derivative of the present invention can be used for a light-emitting element. A carbazole derivative of the present invention has excellent hole injecting property and hole transporting property, and thus, it can be used as a hole transporting material. Specifically, a carbazole derivative of the present invention can be used as a material of a hole injecting layer, a material of a hole transporting layer and a host material of a light-emitting layer which are included in a layer including a luminescent substance.

Therefore, a light-emitting element of the present invention has one feature that the light-emitting element has a layer including a luminescent substance between a pair of electrodes, and the layer including a luminescent substance contains a carbazole derivative of the present invention.

A carbazole derivative of the present invention is preferably included as a hole injecting material, since the carbazole derivative of the present invention has an excellent hole injecting property. In other words, a carbazole derivative of the present invention is preferably used for a layer that is in contact with an electrode serving as an anode.

A carbazole derivative of the present invention has an excellent hole transporting property, and thus, it is preferably used as a hole transporting material. In other words, a carbazole derivative of the present invention is preferably included between an electrode serving as an anode of a pair of electrodes in a light-emitting element, and a layer having a light-emitting function included in the layer having a luminescent layer.

A carbazole derivative of the present invention can be used as a host material of a light-emitting layer. A carbazole derivative of the present invention exhibits light-emission, and thus, it can be used as a light-emitting material. Therefore, a carbazole derivative of the present invention is preferably included in a layer having a light-emitting function of the layer including a luminescent substance.

The category of the present invention includes a light-emitting device having the light-emitting element. Note that the category of the light-emitting device in this specification includes an image display device, a luminescent device, and a light source (including a lighting system). In addition, a module that has a connector such as a flexible printed circuit (FPC), a TAB (Tape Automated Bonding) tape, or a TCP (Tape Carrier Package), attached to a light-emitting device; a module that has a printed wiring board provided at the tip of a TAB tape or a TCP; and a module that has an IC (integrated circuit) directly mounted on a light-emitting element by a COG (Chip On Glass) method, are also all included in the category of the light-emitting device.

A carbazole derivative of the present invention has an excellent hole injecting property, and driving voltage can be decreased by using the carbazole derivative for a hole injecting layer of a light-emitting element as a hole injecting material.

In addition, a carbazole derivative of the present invention has an excellent hole transporting property, and thus, it can be used for a light-emitting element, as a hole transporting material.

A carbazole derivative of the present invention is superior in heat resistance, and thus, a light-emitting element having excellent durability and high heat resistance can be obtained.

In addition, since a light-emitting element of the present invention employs a carbazole derivative of the present invention, the light-emitting element can realize the decrease of driving voltage, the enhancement of an emission efficiency and enhancement of reliability.

A carbazole derivative of the present invention is superior in heat resistance, and thus, a light-emitting element having excellent durability and high heat resistance can be obtained.

Since a light-emitting device of the present invention has a light-emitting element using a carbazole derivative of the present invention, the light-emitting device has a long life time. Thus, a light-emitting device having high reliability can be provided.

### BRIEF DESCRIPTION OF THE DRAWINGS

In the accompanying drawings:

FIG. 1 shows a light-emitting element according to an aspect of the present invention;

FIG. 2 shows a light-emitting element according to an aspect of the present invention;

FIG. 3 shows a light-emitting element according to an aspect of the present invention;

FIGS. 4A and 4B each show a light-emitting device;

FIGS. 5A to 5E each show an electronic device;

FIG. 6 is a ¹H-NMR chart of 3-[N-(9-phenylcarbazole-3-yl)-N-phenylamino]-9-phenylcarbazole which is a carbazole derivative of the present invention:

FIG. 7 is a ¹H-NMR chart of 3-[N-(9-phenylcarbazole-3-yl)-N-phenylamino]-9-phenylcarbazole which is a carbazole derivative of the present invention:

FIG. 8 is a graph showing absorption spectra of 3-[N-(9-phenylcarbazole-3-yl)-N-phenylamino]-9-phenylcarbazole which is a carbazole derivative of the present invention:

FIG. 9 is a graph showing light-emission spectra of 3-[N-(9-phenylcarbazole-3-yl)-N-phenylamino]-9-phenylcarbazole which is a carbazole derivative of the present invention:

FIG. 10 is a ¹H-NMR chart of 3,6-bis[N-(9-phenylcarbazole-3-yl)-N-phenylamino]-9-phenylcarbazole which is a carbazole derivative of the present invention:

FIG. 11 is a ¹H-NMR chart of 3,6-bis[N-(9-phenylcarbazole-3-yl)-N-phenylamino]-9-phenylcarbazole which is a carbazole derivative of the present invention:

FIG. 12 is a graph showing absorption spectra of 3,6-bis[N-(9-phenylcarbazole-3-yl)-N-phenylamino]-9-phenylcarbazole which is a carbazole derivative of the present invention:

FIG. 13 is a graph showing light-emission spectra of 3,6-bis[N-(9-phenylcarbazole-3-yl)-N-phenylamino]-9-phenylcarbazole which is a carbazole derivative of the present invention:

FIG. 14 is a graph showing results in thermogravimetric measurement of 3-[N-(9-phenylcarbazole-3-yl)-N-phenylamino]-9-phenylcarbazole which is a carbazole derivative of the present invention:

FIG. 15 is a graph showing results in thermogravimetric measurement of 3,6-bis[N-(9-phenylcarbazole-3-yl)-N-phenylamino]-9-phenylcarbazole which is a carbazole derivative of the present invention:

FIG. 16 is a graph showing C-V characteristics of 3-[N-(9-phenylcarbazole-3-yl)-N-phenylamino]-9-phenylcarbazole which is a carbazole derivative of the present invention:

FIG. 17 is a graph showing C-V characteristics of 3,6-bis[N-(9-phenylcarbazole-3-yl)-N-phenylamino]-9-phenylcarbazole which is a carbazole derivative of the present invention:

FIG. 18 is a graph showing results obtained by a differential scanning calorimetry analysis of 3-[N-(9-phenylcarbazole-3-yl)-N-phenylamino]-9-phenylcarbazole which is a carbazole derivative of the present invention:

FIG. 19 is a graph showing results obtained by a differential scanning calorimetry analysis of 3,6-bis[N-(9-phenylcarbazole-3-yl)-N-phenylamino]-9-phenylcarbazole which is a carbazole derivative of the present invention:

FIG. 20 is a ¹H-NMR chart of 3-(N-phenylamino)-9-phenylcarbazole;

FIG. 21 is a ¹H-NMR chart of 3-(N-phenylamino)-9-phenylcarbazole;

FIG. 22 is a ¹H-NMR chart of 3-[N-(1-naphtyl)amino]-9-phenylcarbazole which is a carbazole derivative of the present invention:

FIG. 23 is a ¹H-NMR chart of 3-[N-(1-naphtyl)amino]-9-phenylcarbazole which is a carbazole derivative of the present invention:

FIG. 24 is a ¹H-NMR chart of 3-[N-(1-naphtyl)-N-(9-phenylcarbazole-3-yl)amino]-9-phenylcarbazole which is a carbazole-derivative of the present invention:

FIG. 25 is a ¹H-NMR chart of 3-[N-(1-naphtyl)-N-(9-phenylcarbazole-3-yl)amino]-9-phenylcarbazole which is a carbazole derivative of the present invention:

FIG. 26 is a graph showing results in thermogravimetric measurement of 3-[N-(1-naphtyl)-N-(9-phenylcarbazole-3-yl)amino]-9-phenylcarbazole which is a carbazole derivative of the present invention:

FIG. 27 is a graph showing absorption spectra of 3-[N-(1-naphtyl)-N-(9-phenylcarbazole-3-yl)amino]-9-phenylcarbazole which is a carbazole derivative of the present invention:

FIG. 28 is a graph showing light-emission spectra of 3-[N-(1-naphtyl)-N-(9-phenylcarbazole-3-yl)amino]-9-phenylcarbazole which is a carbazole derivative of the present invention:

FIG. 29 is a graph showing C-V characteristics of 3-[N-(1-naphtyl)-N-(9-phenylcarbazole-3-yl)amino]-9-phenylcarbazole which is a carbazole derivative of the present invention:

FIG. 30 is a graph showing results obtained by a differential scanning calorimetry analysis of 3-[N-(1-naphtyl)-N-(9-phenylcarbazole-3-yl)amino]-9-phenylcarbazole which is a carbazole derivative of the present invention:

FIGS. 31A and 31B are each a ¹H-NMR chart of 3-{N-[9-(4-biphenylyl)carbazole-3-yl]-N-phenylamino}-9-(4-biphenylyl)carbazole which is a carbazole derivative of the present invention:

FIGS. 32A and 32B are each a ¹³C-NMR chart of 3-{N-[9-(4-biphenylyl)carbazole-3-yl]-N-phenylamino}-9-(4-biphenylyl)carbazole which is a carbazole derivative of the present invention:

FIG. 33 is a graph showing absorption spectra of 3-{N-[9-(4-biphenylyl)carbazole-3-yl]-N-phenylamino}-9-(4-biphenylyl)carbazole which is a carbazole derivative of the present invention:

FIG. 34 is a graph showing light-emission spectra of 3-{N-[9-(4-biphenylyl)carbazole-3-yl]-N-phenylamino}-9-(4-biphenylyl)carbazole which is a carbazole derivative of the present invention:

FIGS. 35A and 35B each are a ¹H-NMR chart of 3,6-dibromo-9-(4-biphenylyl)carbazole;

FIGS. 36A and 36B each are a ¹³C-NMR chart of 3,6-dibromo-9-(4-biphenylyl)carbazole;

FIGS. 37A and 37B each are a ¹H-NMR chart of 3,6-bis[N-(1-naphtyl)-N-(9-phenylcarbazole-3-yl)amino]-9-(4-biphenylyl)carbazole;

FIGS. 38A and 38B each are ¹³C-NMR chart of 3,6-bis[N-(1-naphtyl)-N-(9-phenylcarbazole-3-yl)amino]-9-(4-biphenylyl)carbazole;

FIG. 39 is a graph showing absorption spectra of 3,6-bis[N-(1-naphtyl)-N-(9-phenylcarbazole-3-yl)amino]-9-(4-biphenylyl)carbazole;

FIG. 40 is a graph showing light-emission spectra of 3,6-bis[N-(1-naphtyl)-N-(9-phenylcarbazole-3-yl)amino]-9-(4-biphenylyl)carbazole;

FIG. 41 shows a light-emitting element in Examples;

FIG. 42 is a graph showing current density-luminance characteristics of the light-emitting element which is formed in Example 8;

FIG. 43 is a graph showing voltage-luminance characteristics of the light-emitting element which is formed in Example 8;

FIG. 44 is a graph showing current density-luminance characteristics of the light-emitting element which is formed in Example 9;

FIG. 45 is a graph showing voltage-luminance characteristics of the light-emitting element which is formed in Example 9;

FIG. 46 is a graph showing current density-luminance characteristics of the light-emitting element which is formed in Example 10;

FIG. 47 is a graph showing voltage-luminance characteristics of the light-emitting element which is formed in Example 10;

FIG. 48 is a DSC chart of 3-{N-[9-(4-biphenylyl)carbazole-3-yl]-N-phenylamino}-9-(4-biphenylyl)carbazole; and

FIG. 49 is a DSC chart of 3,6-bis[N-(1-naphtyl)-N-(9-phenylcarbazole-3-yl)amino]-9-(4-biphenylyl)carbazole.

### BEST MODE FOR CARRYING OT THE INVENTION

### Embodiment Modes

Hereinafter, the embodiment modes of the present invention will be described with reference to the accompanying drawings. The present invention can be carried out in many different modes without being limited to the following description, and it is easily understood by those skilled in the art that modes and details herein disclosed can be modified in various ways without departing from the spirit and the scope of the present invention. It should be noted that the present invention should not be interpreted as being limited to the description of the embodiment modes to be given below.

As for a pair of electrodes of a light-emitting element according to the present invention, when a voltage is applied such that a potential of one electrode thereof is higher than that of the other electrode, light is emitted. At this time, one electrode having a higher potential, is referred to as an electrode serving as an anode, and the other electrode having a lower potential, is referred to as an electrode serving as a cathode.

### Embodiment Mode 1

A carbazole derivative of the present invention is a carbazole derivative having a structure represented by the general formula (1).

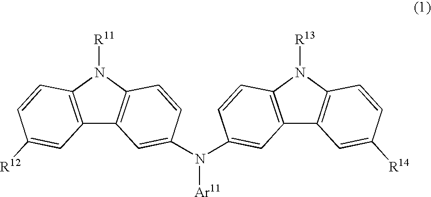

wherein R¹¹ and R¹³ may each the same or different, and represent hydrogen, an alkyl group having carbon atoms 1 to 6, an aryl group having carbon atoms 6 to 25, a hetero aryl group having carbon atoms 5 to 9, an aryl alkyl group, or an acyl group having carbon atoms 1 to 7; Ar¹¹ represents an aryl group having carbon atoms 6 to 25, or a hetero aryl group having carbon atoms 5 to 9; R¹² represents hydrogen, an alkyl group having carbon atoms 1 to 6, or an aryl group having carbon atoms 6 to 12; R¹⁴ represents hydrogen, an alkyl group having carbon atoms 1 to 6, an aryl group having carbon atoms 6 to 12, or a substituent represented by the general formula (2); in the substituent represented by the general formula (2), R¹⁵ represents hydrogen, an alkyl group having carbon atoms 1 to 6, an aryl group having carbon atoms 6 to 25, a hetero aryl group having carbon atoms 5 to 9, an aryl alkyl group, or an acyl group having carbon atoms 1 to 7; Ar¹² represents an aryl group having carbon atoms 6 to 25, or a hetero aryl group having carbon atoms 5 to 9; and R¹⁶ represents hydrogen, an alkyl group having carbon atoms 1 to 6, or an aryl group having carbon atoms 6 to 12.

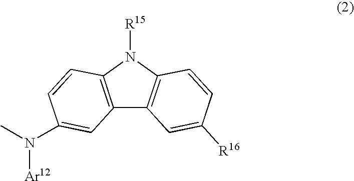

As an alkyl group having carbon atoms 1 to 6, specifically, methyl, ethyl, n-propyl, n-butyl, n-hexyl, and the like, are given. In addition, an alkyl group having a branch, such as iso-propyl or tert-butyl, may be used.

As an aryl group having carbon atoms 6 to 25, specifically, phenyl, 4-biphenylyl, 1-naphthyl, 2-naphthyl, 9-anthryl, 9-phenantryl, 1-pyrenyl, 9,9′-dimethyl-2-fluorenyl, spiro-9-9′-bifluorene-2-yl, and the like, are given. In addition, an aryl group having a substituent such as m-tolyl, p-tolyl, 2-fluoro phenyl, 3-fluoro phenyl, 4-fluoro phenyl may be used.

As a hetero aryl group having carbon atoms 5 to 9, specifically, 2-pyridyl, 8-quinolyl, 3-quinolyl and the like are given.

As an aryl alkyl group, benzyl and the like are given.

As an acyl group having carbon atoms 1 to 7, specifically, acetyl, benzoyl, a propionyl, and the like, are nominated.

In the above formula (1), either R¹¹ or R¹³ is preferably an aryl group having carbon atoms 6 to 25, or a hetero aryl group having carbon atoms 5 to 9. More preferably, R¹¹ and R¹³ are preferably an aryl group having carbon atoms 6 to 25, or a hetero aryl group having carbon atoms 5 to 9. A substituent binding with nitrogen of a carbazole skeleton employs an aryl group having carbon atoms 6 to 25, or a heteroaryl group having carbon atoms 5 to 9, and thus, an effect that a carrier transporting property is enhanced, can be obtained.

In addition, in the general formula (1), R¹² is preferably hydrogen, tert-butyl, phenyl, or biphenyl.

In the general formula (1), R¹⁴ is preferably hydrogen, tert-butyl, phenyl, or biphenyl.

In the general formula (1), R¹⁴ is preferably a substituent represented by the general formula (2). A carbazole derivative having higher heat resistance can be obtained by using the substituent represented by the general formula (2) in R⁴. In the general formula (2), R¹⁵ is preferably an aryl group having carbon atoms 6 to 25, or a hetero aryl group having carbon atoms 5 to 9. The substituent which binds with nitrogen of the carbazole skeleton employs an aryl group having carbon atoms 6 to 25 or a hetero aryl group having carbon atoms 5 to 9, and thus, an effect that a carrier transporting property is enhanced, can be obtained. In addition, in the general formula (2), R¹⁶ is preferably hydrogen, tert-butyl, or biphenyl.

Another structure of the present invention is a carbazole derivative having a structure represented by the general formula (3).

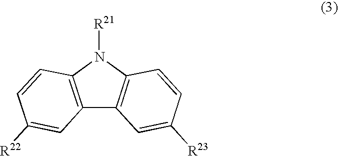

wherein R²¹ represents hydrogen, an alkyl group having carbon atoms 1 to 6, an aryl group having carbon atoms 6 to 25, a heteroaryl group having carbon atoms 5 to 9, an arylalkyl group, or an acyl group having carbon atoms 1 to 7; R²² represents hydrogen, an alkyl group having carbon atoms 1 to 6, or an aryl group having carbon atoms 6 to 12; R²³ represent a substituent represented by the general formula (4); in the substituent represented by the general formula (4), R²⁴ represents hydrogen, an alkyl group having carbon atoms 1 to 6, an aryl group having carbon atoms 6 to 25, a heteroaryl group having carbon atoms 5 to 9, an arylalkyl group, or an acyl group having carbon atoms 1 to 7; Ar²¹ represents an aryl group having carbon atoms 6 to 25, or a heteroaryl group having carbon atoms 5 to 9; and R²⁵ represents hydrogen, an alkyl group having carbon atoms 1 to 6, or a heteroaryl group having carbon atoms 6 to 12.

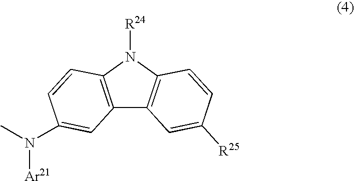

In the above structure, R²² represents hydrogen, tert-butyl, phenyl, or biphenyl.

Another structure of the present invention is a carbazole derivative having a structure represented by the general formula (5).

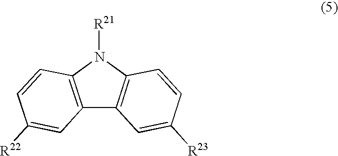

wherein R²¹ represents hydrogen, an alkyl group having carbon atoms 1 to 6, an aryl group having carbon atoms 6 to 25, a heteroaryl group having carbon atoms 5 to 9, an arylalkyl group, or an acyl group having carbon atoms 1 to 7; R²² and R²³ represent a substituent represented by the general formula (6); in the substituent represented by the general formula (6), R²⁴ represents hydrogen, an alkyl group having carbon atoms 1 to 6, an aryl group having carbon atoms 6 to 25, a heteroaryl group having carbon atoms 5 to 9, an arylalkyl group, or an acyl group having carbon atoms 1 to 7; Ar²¹ represents an aryl group having carbon atoms 6 to 25, or a heteroaryl group having carbon atoms 5 to 9; and R²⁵ represents hydrogen, an alkyl group having carbon atoms 1 to 6, or a heteroaryl group having carbon atoms 6 to 12.

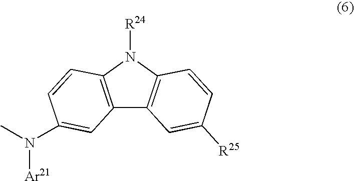

In the above structure, R²⁵ is preferably hydrogen, tert-butyl, phenyl, or biphenyl.

In the above structure, R²⁴ is preferably an aryl group having carbon atoms 6 to 25, or a hetero aryl group having carbon atoms 5 to 9.

In the above structure, R²¹ is preferably an aryl group having carbon atoms 6 to 25 or a hetero aryl group having carbon atoms 5 to 9.

The substituent which binds with nitrogen of the carbazole skeleton employs an aryl group having carbon atoms 6 to 25 or a hetero aryl group having carbon atoms 5 to 9, and thus, an effect that a carrier transporting property is enhanced, can be obtained.

An alkyl group having carbon atoms 1 to 6 or an aryl group having carbon atoms 6 to 12 is preferably bound to the sixth position of the carbazole skeleton. As a result of using a substituent of an alkyl group having carbon atoms 1 to 6 or an aryl group having carbon atoms 6 to 12, for the sixth position of the carbazole skeleton, the carbazole skeleton becomes chemically stable, and a secondary reaction can be suppressed.

Another structure of the present invention is a carbazole skeleton having a structure represented by the general formula (7).

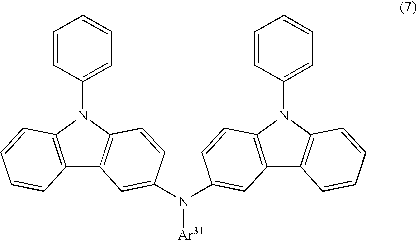

wherein Ar³¹ represents phenyl or naphthyl.

Another structure of the present invention is a carbazole skeleton having a structure represented by the general formula (8).

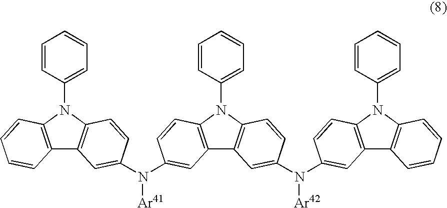

wherein Ar⁴¹ and Ar⁴² may be the same or different and represent phenyl or naphthyl.

As a specific example of carbazole derivatives of the present invention, carbazole derivatives represented by the following structural formulae (9) to (71), can be provided. Note that the present invention is not limited to these derivatives.

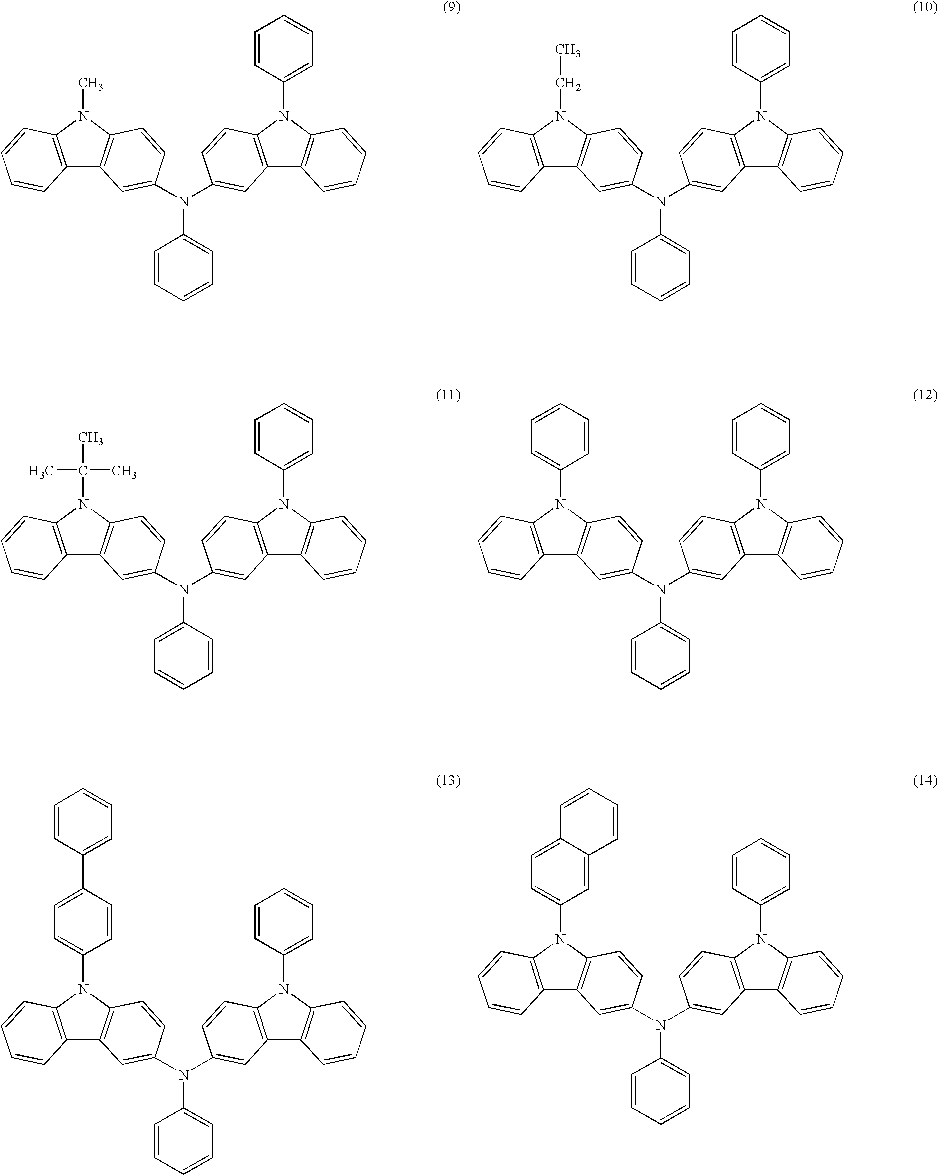

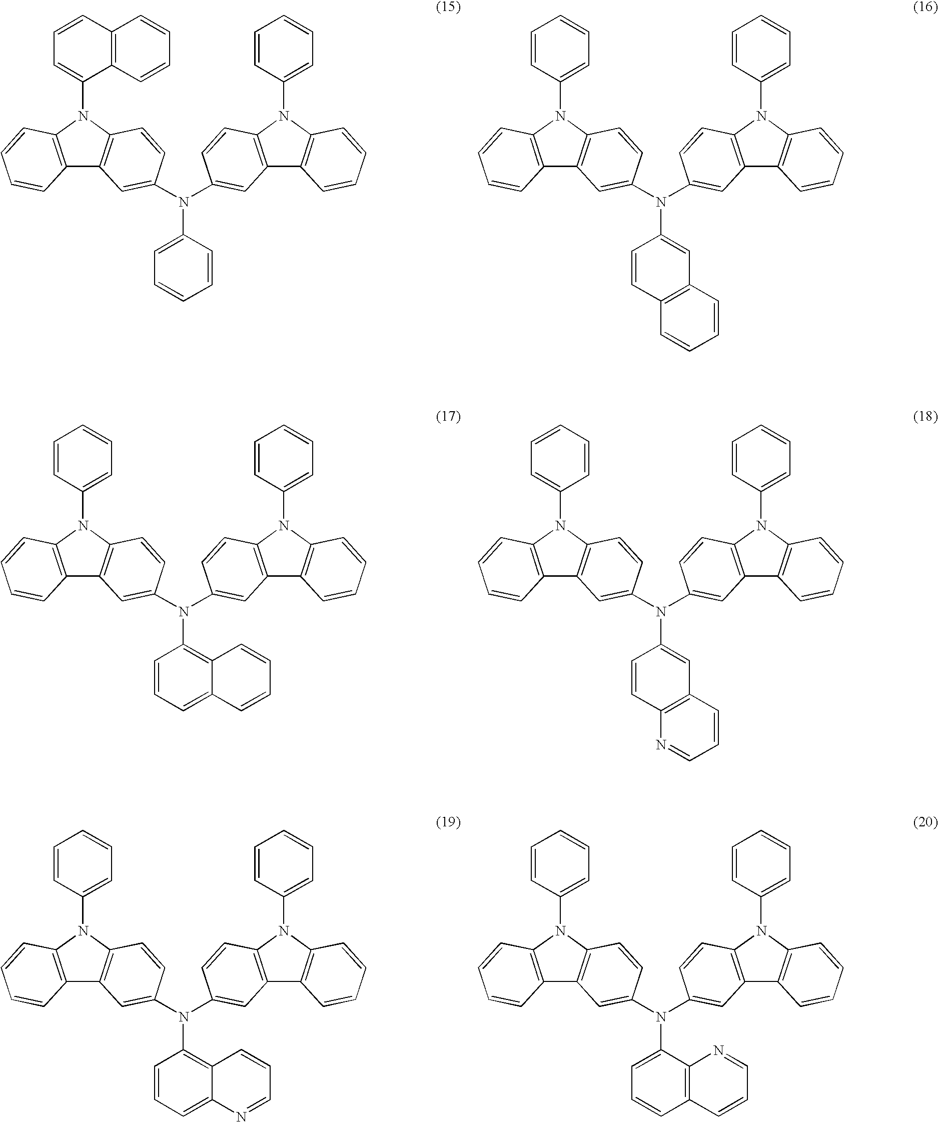

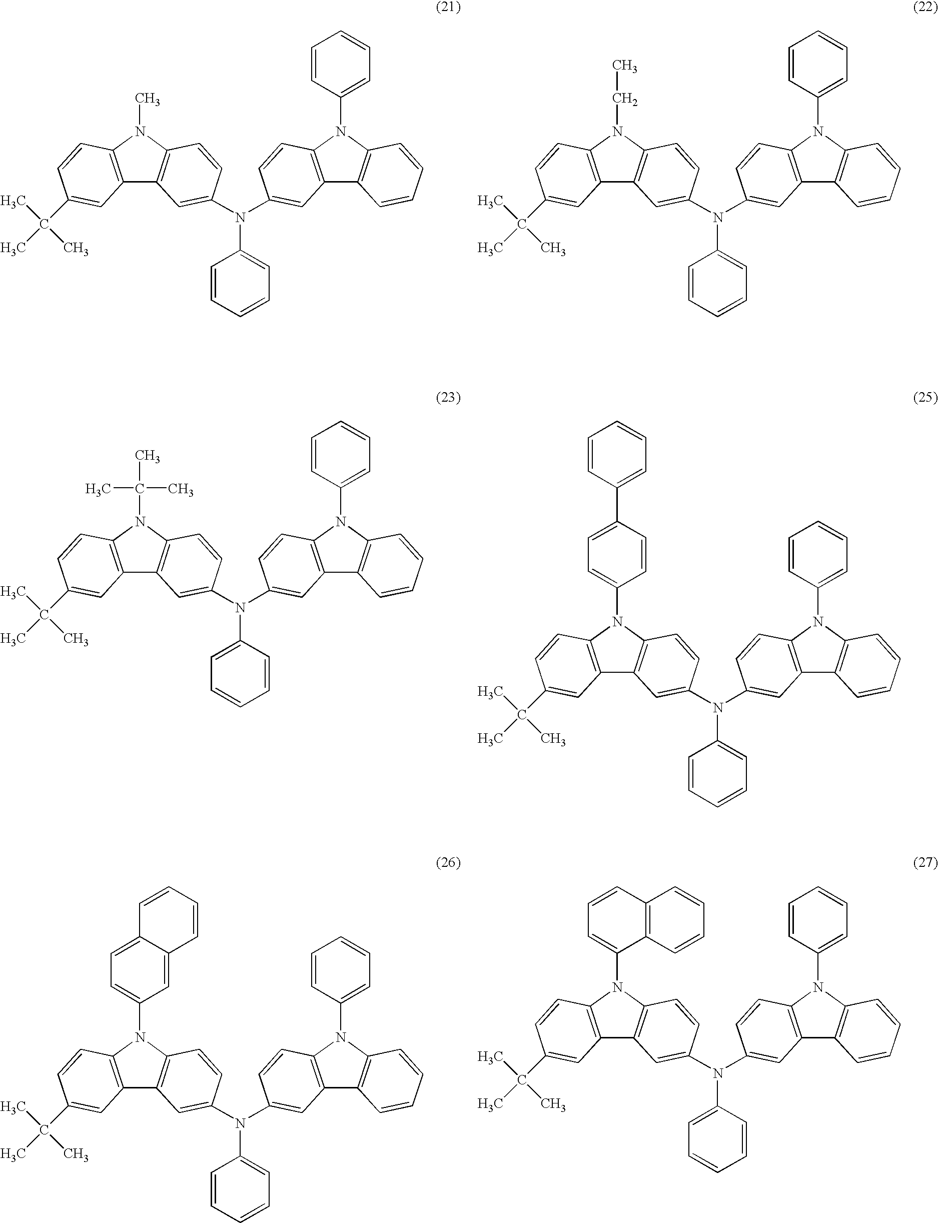

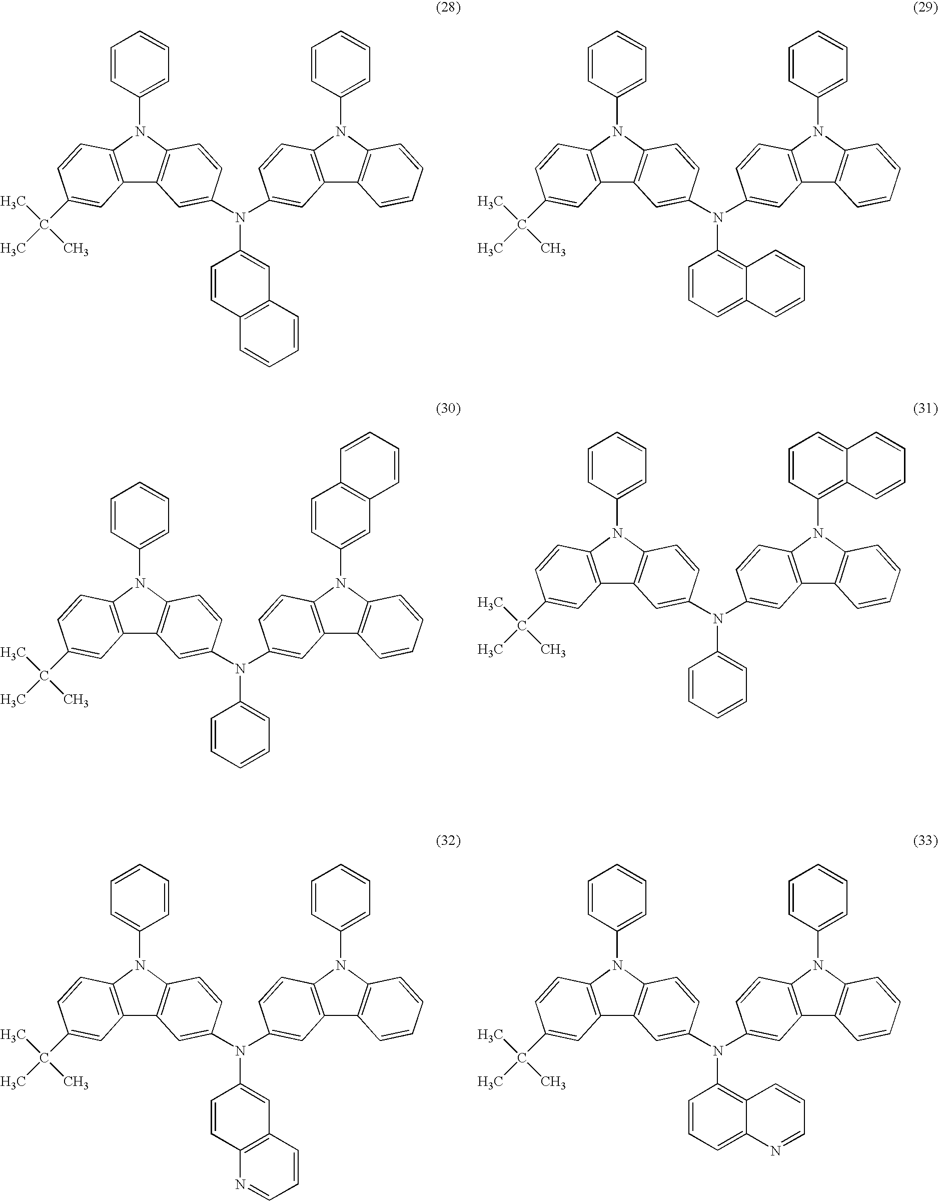

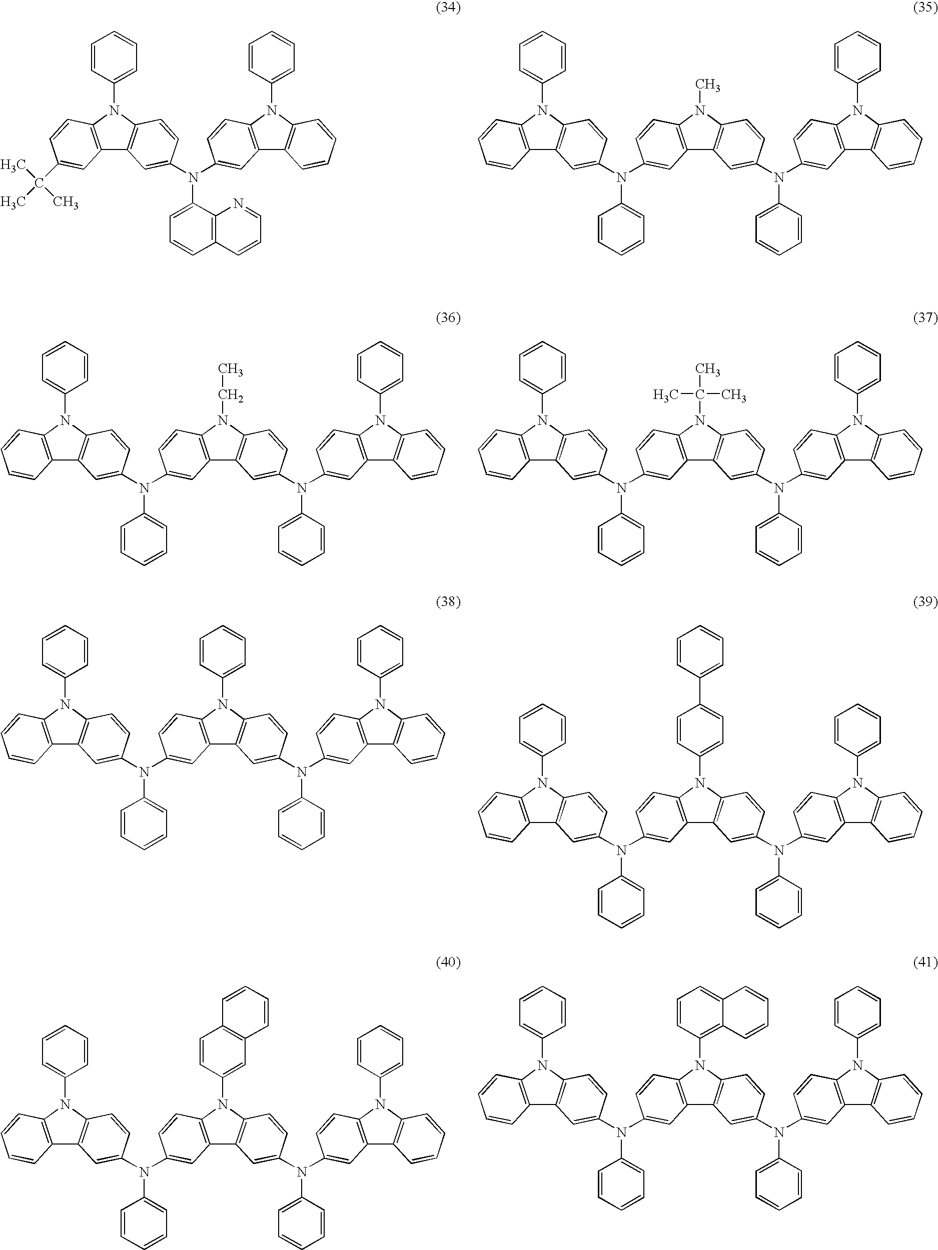

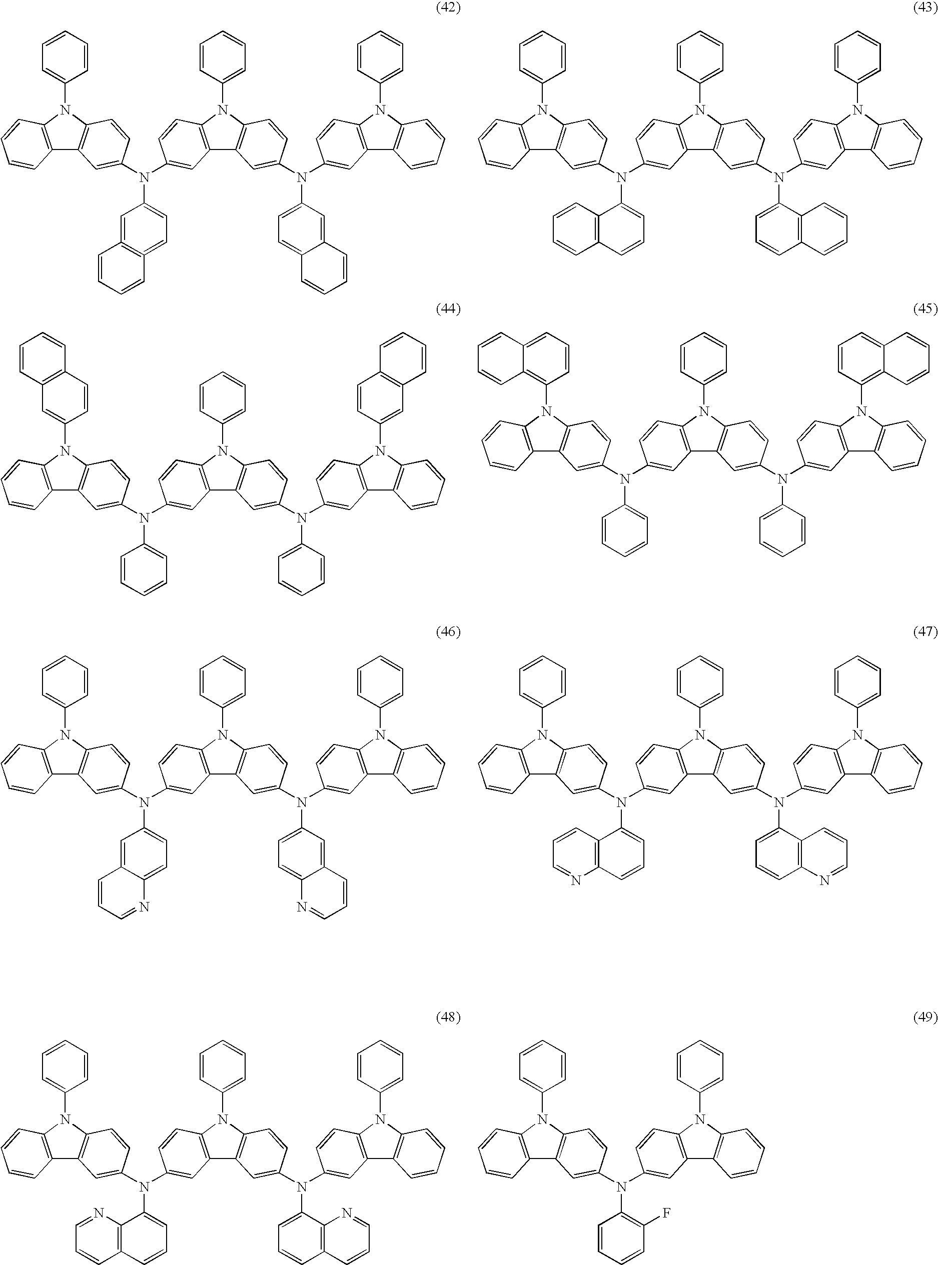

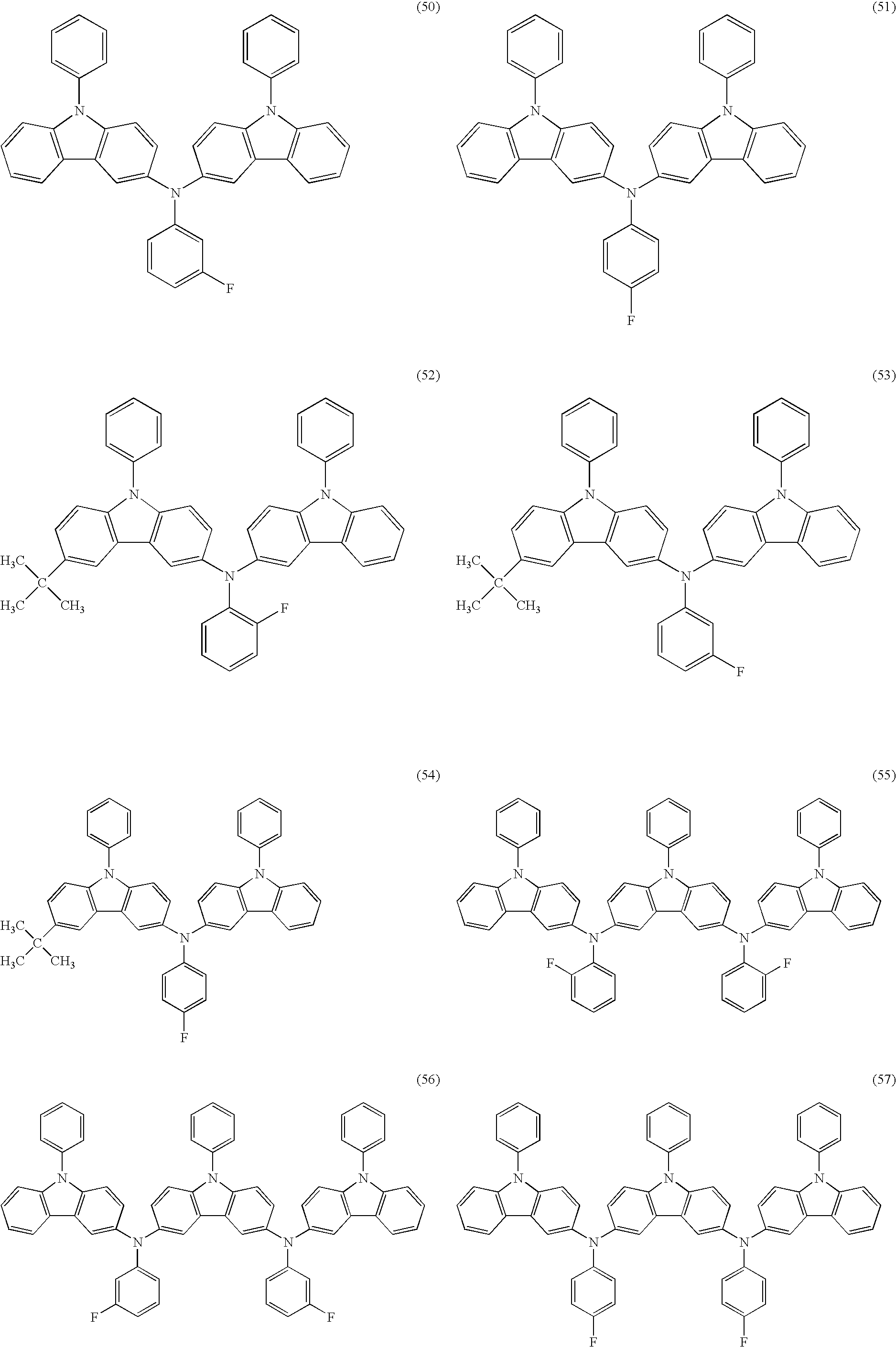

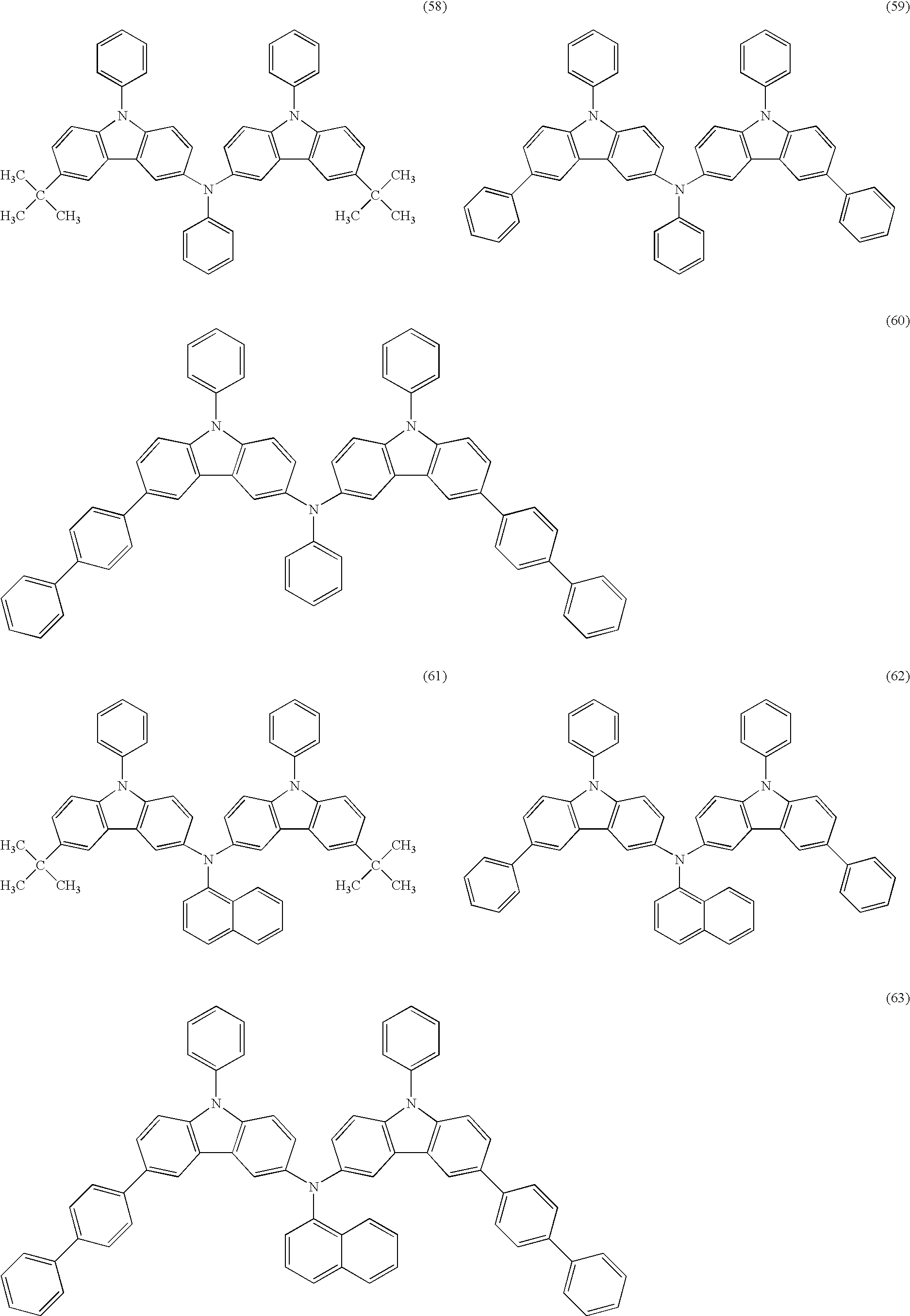

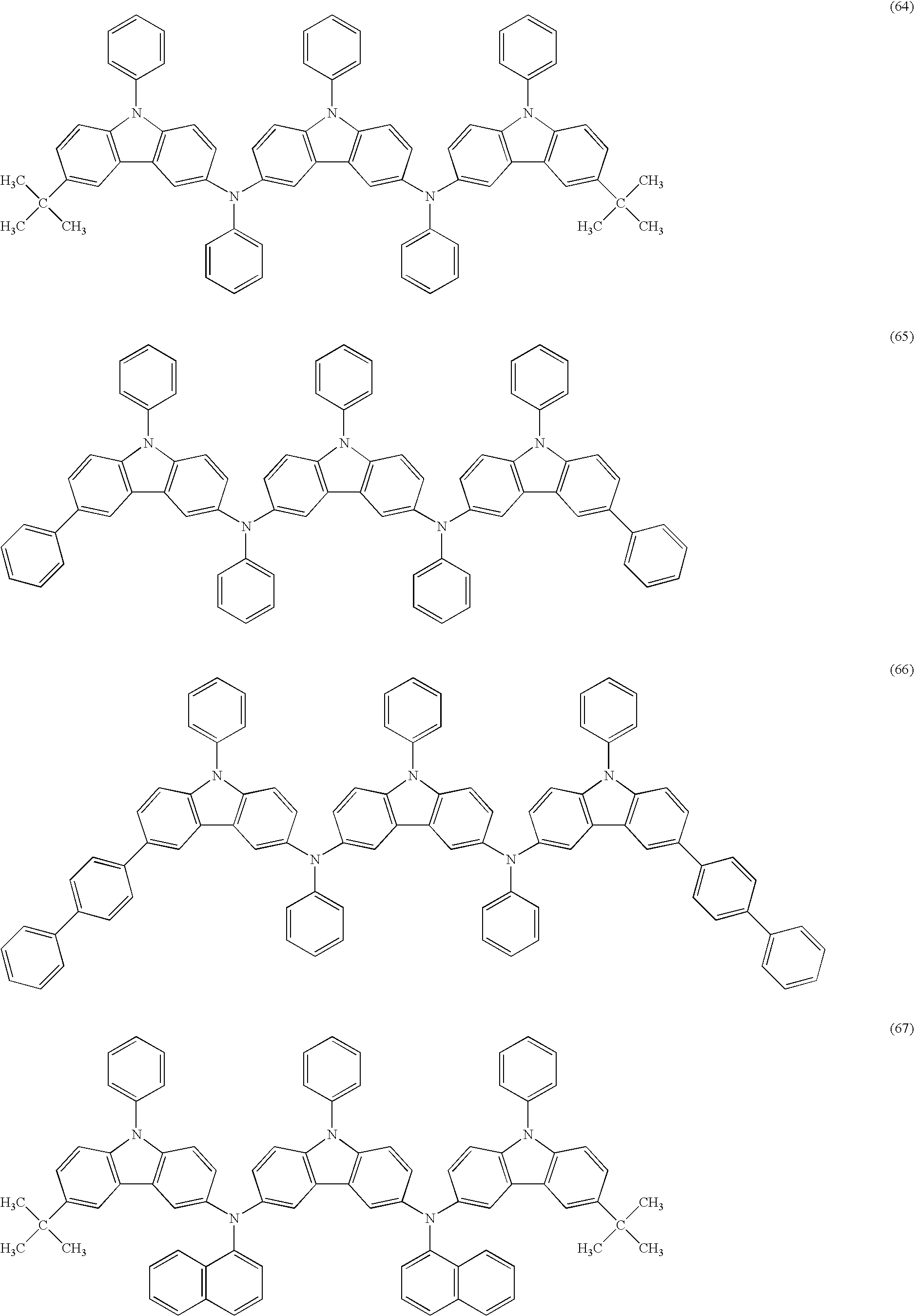

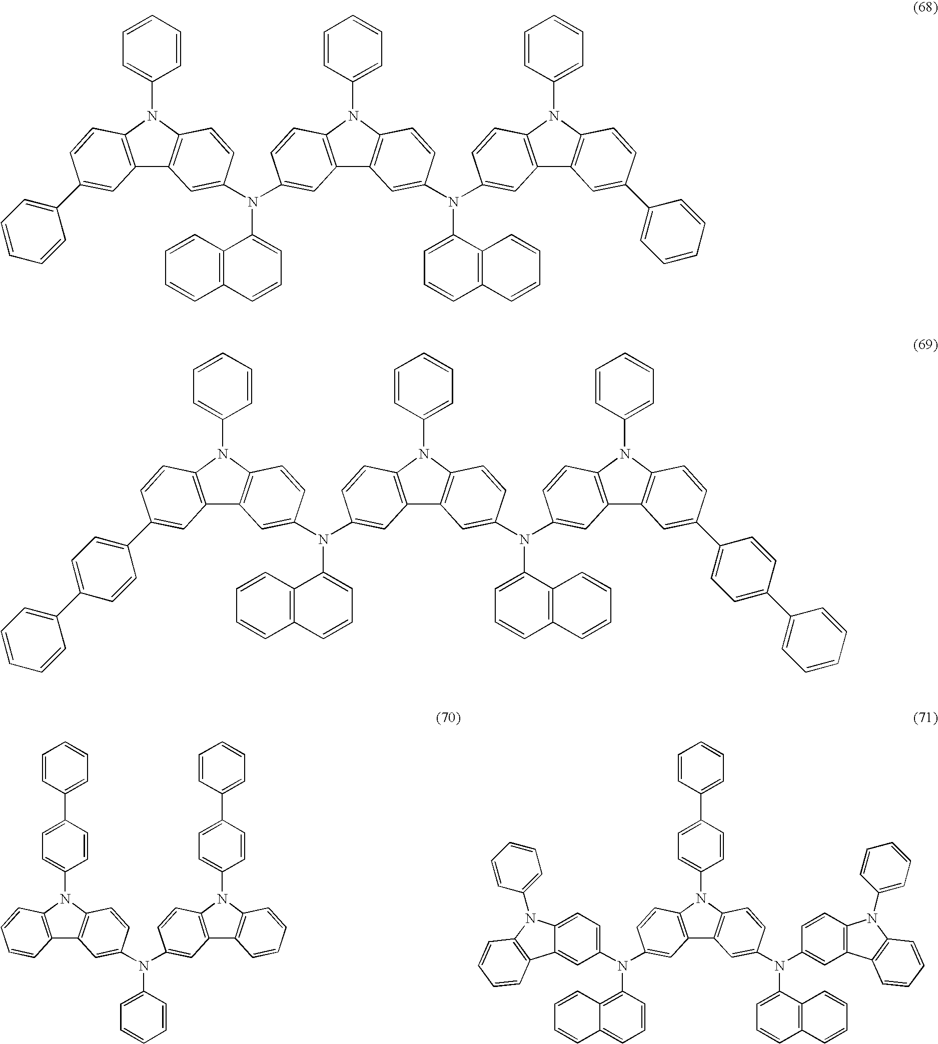

The carbazole derivatives represented by the structural formulae (9) to (20) have hydrogen in R¹² of the general formula (1), while the carbazole derivatives represented by the structural formulae (21) to (34) have an alkyl group in R¹² in the general formula (1).

The carbazole derivatives represented by the structural formulae (35) to (48) each have a structure in which the same substituents are bound with a carbazole skeleton, and the synthesis thereof can be conducted more easily than that of a carbazole derivative having a structure in which different substituents are bound. In other words, when R²² and R²³ in the general formula (3) have the same structure, which is represented by the general formula (4), the same substituents may be bound with the carbazole skeleton, and thus, the synthesis can be conducted more easily.

A carbazole derivative of the present invention may have fluorine as shown by the structural formulae (49) to (57).

In addition, as shown by the structural formulae (58) to (69), an alkyl group having carbon atoms 1 to 6 or an aryl group having carbon atoms 6 to 12 is preferably bound to the sixth position of the carbazole skeleton. As a result of using a substituent of an alkyl group having carbon atoms 1 to 6 or an aryl group having carbon atoms 6 to 12, for the sixth position of the carbazole skeleton, the carbazole skeleton becomes chemically stable, and a secondary reaction can be suppressed.

As a synthesis method of a carbazole derivative of the present invention, various reactions can be applied. For example, there are methods represented by reaction schemes (A-1) and (A-2). However, the synthesis method of a carbazole derivative of the present invention is not limited thereto.

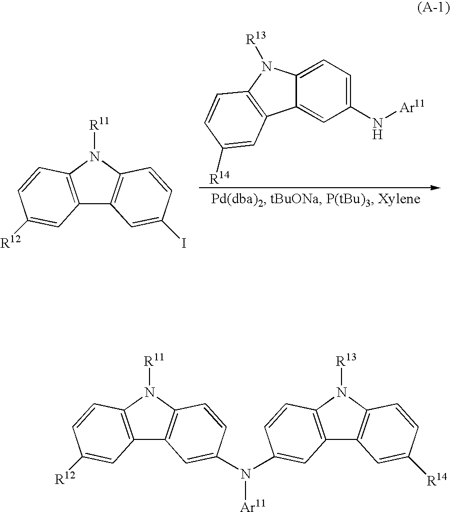

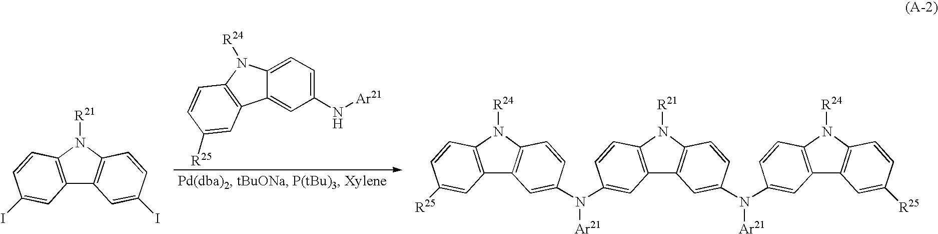

### Embodiment Mode 2

Embodiment Mode 2 will describe a light-emitting element using a carbazole derivative shown in Embodiment Mode 1.

A light-emitting element of the present invention has a structure in which a layer including a luminescent substance is interposed between a pair of electrode. The structure of the light-emitting element is not especially limited, and can adopt a known structure appropriately in accordance with the purpose.

Since a carbazole derivative of the present invention is superior in a hole injecting property, it is preferably used for a hole injecting layer as a hole injecting material. In addition, since a carbazole derivative of the present invention is also superior in a hole transporting property, it can be used as a hole transporting material. Specifically, the carbazole derivative of the present invention can be used as a material of a hole transporting layer and a host material of a light-emitting layer which are included in the layer including a luminescent substance. A carbazole derivative of the present invention can emit light of blue based light or the like, and thus, it can be used as a light-emitting material. Specifically, the carbazole derivative of the present invention can be used as a guest material of a light-emitting layer.

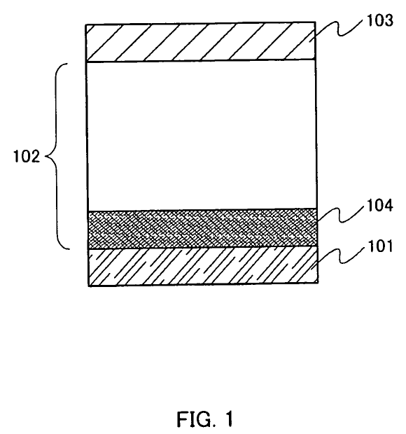

FIG. 1 schematically shows an element structure of a light-emitting element of the present invention as one example. In this embodiment mode, a case that a carbazole derivative of the present invention is used for a hole injecting layer, is described.

A light-emitting element shown in FIG. 1 has a structure in which a layer including a luminescent substance 102 is interposed between a first electrode 101 and a second electrode 103. In this embodiment mode, the first electrode 101 serves as an anode and the second electrode 103 serves as a cathode. A layer which is contact with the anode 104 included in the layer including a luminescent substance 102 includes a carbazole derivative. In other words, the layer including a carbazole derivative of the present invention serves as a hole injecting layer.

As the anode, a known material can be used, and a metal, an alloy, a conductive compound, or a mixture thereof having a high work function (specifically, 4.0 eV or more) is preferably used. Specifically, indium tin oxide (ITO), or indium tin oxide containing silicon, indium oxide containing zinc oxide (ZnO) of 2 to 20 wt % (IZO), indium oxide-tin oxide (IWZO) containing tungsten oxide and zinc oxide, and the like can be given. These conductive metal oxide films are usually formed by a sputtering method. For example, IZO (indium oxide containing zinc oxide (ZnO)) can be formed by a sputtering method using a target in which zinc oxide of 1 to 20 wt % is contained in indium oxide. In addition, indium oxide-tin oxide (IWZO) containing tungsten oxide and zinc oxide can be formed by a sputtering method using a target in which tungsten oxide of 0.5 to 5 wt % and zinc oxide of 0.1 to 1 wt % are included in indium oxide. Additionally, gold (Au), platinum (Pt), nickel (Ni), tungsten (W), chromium (Cr), molybdenum (Mo), iron (Fe), cobalt (Co), copper (Cu), palladium (Pd), a nitride of a metal material (such as titanium nitride) and the like, can be used.

On the other hand, a known material can be used as a cathode, and a metal, an alloy, a conductive compound, a mixture of them, or the like having a high work function (specifically, 3.8 eV or less) is preferably used. Specifically, a metal belonging to Group 1 or 2 of the periodic table of the elements, that is, an alkali metal such as lithium (Li) or cesium (Cs), an alkali-earth metal such as magnesium (Mg), calcium (Ca), or strontium (Sr), and an alloy (such as MgAg and AlLi) including the above metals, a rare-earth metal such as europium (Eu) or ytterbium (Yb), an alloy including the rare-earth metal, or the like can be used. However, by using an electron injecting layer which has a high electron injecting property, a material having a higher work function, that is, a material that is normally used as the anode, can also be used to form the cathode. For example, a metal such as Al, Ag, or a conductive inorganic compound such as ITO can be used to form the cathode.

For the layer including a luminescent substance 102, known materials can be used, and any of low molecular weight materials or high molecular weight materials can be used. An organic material including partially an inorganic material, as well as an organic material itself can be used as a material for forming the layer including a luminescent substance 102. Further, the layer including a luminescent substance is formed by appropriately combining a hole injecting layer, a hole transporting layer, a hole blocking layer, a light-emitting layer, an electron transporting layer, an electron injecting layer, and the like. The layer including a luminescent substance may be a single layer or have a stacked structure of a plurality of layers.

The layer including a luminescent substance can be formed by a method such as an evaporation method, an ink-jet method, a spin-coating method, or a dip-coating method, regardless of the type of the method, i.e., a wet type method or a dry type method.

Hereinafter, specific materials used for a hole injecting layer, a hole transporting layer, a light-emitting layer, an electron transporting layer, and an electron injecting layer are described.

As a hole injecting material forming a hole injecting layer, the carbazole derivative according to the present invention can be used. The carbazole derivative according to the present invention has an excellent hole injecting property. By using the carbazole derivative according to the present invention as a hole injecting material, it is possible to reduce the driving voltage of the light-emitting element.

An aromatic amine based compound (in other words, a compound having a benzene ring-nitrogen bond) is preferably used as a hole transporting material for forming the hole transporting layer. Examples of materials that are widely used include, for example, N,N′-bis(3-methylphenyl)-N,N′-diphenyl-[1,1′-biphenyl]-4,4′-diamine (hereinafter referred to as TPD), derivatives thereof such as 4,4′-bis[N-(1-naphthyl)-N-phenyl-amino]-biphenyl (hereinafter referred to as α-NPD), and star burst aromatic amine compounds such as 4,4′,4″-tris(N-carbazolyl)-triphenylamine (hereinafter referred to as TCTA), 4,4′,4″-tris(N,N-diphenyl-amino)-triphenylamine (hereinafter referred to as TDATA) and 4,4′,4″-tris[N-(3-methylphenyl)-N-phenyl-amino]-triphenylamine (referred to as MTDATA).

In addition, the carbazole derivative according to the present invention has an excellent hole transporting property, and thus can be used as a hole transporting material.

As a light-emitting material forming the light-emitting layer, specifically, various fluorescent pigments are effective in addition to metal complexes such as tris(8-quinolinolato)aluminum (hereinafter referred to as Alq₃), tris(4-methyl-8-quinolinolato)aluminum (hereinafter referred to as Almq₃), bis(10-hydroxybenzo[h]-quinolinolato)beryllium (hereinafter referred to as BeBq₂), bis(2-methyl-8-quinolinolato)-(4-hydroxy-biphenyl)-aluminum (hereinafter referred to as BAlq), bis[2-(2-hydroxyphenyl)-benzoxazolate]zinc (hereinafter referred to as Zn(BOX)₂), and bis[2-(2-hydroxyphenyl)-benzothiazolate]zinc (hereinafter referred to as Zn(BTZ)₂).

When the light-emitting layer is formed in combination with a guest material, concretely as the guest material, triplet light-emitting material materials (phosphorescent materials) such as bis(2-(2′-benzothienyl)pyridinato-N,C^{3′})(acetylacetonate)iridium (Ir(btp)₂(acac)) can be used, in addition to singlet light-emitting materials (fluorescent materials) such as 4-(dicyanomethylene)-2-methyl-6-(p-dimethylaminostyryl)-4H-pyran (DCM 1), 4-(dicyanomethylene)-2-methyl-6-(julolidine-4-yl-vinyl)-4H-pyran (DCM2), N,N-dimethylquinacridon (DMQd), 9,10-diphenylanthracene, 5,12-diphenyltetracene (DPT), coumarin 6, perylene, and rubrene.

The carbazole derivative of the present invention is a light-emitting material which can emit blue light or the like. Therefore, it is possible to use a carbazole derivative of the present invention as a guest material of the light-emitting layer. Since the carbazole derivative according to the present invention can provide luminescent color other than blue light, a carbazole derivative of the present invention is not limited to the light-emitting element that emits blue light.

In addition, a carbazole derivative of the present invention has an excellent hole transporting property, and thus can be used as a host material of the light-emitting layer.

As an electron transporting material forming the electron transporting layer, the metal complexes mentioned above such as Alq₃, tris(4-methyl-8-quinolinolato)aluminum (Almq₃), bis(2-methyl-8-quinolinolato)-4-phenylphenolato-aluminum (BAlq), tris(8-quinolinolato)gallium (Gaq₃), bis(2-methyl-8-quinolinolato)-4-phenylphenolate-gallium (BGaq), bis(10-hydroxybenzo[h]-quinolinolato)beryllium (BeBq₂), bis[2-(2-hydroxyphenyl)-benzoxazolate]zinc (Zn(BOX)₂), and bis[2-(2-hydroxyphenyl)-benzothiazolate]zinc (Zn(BTZ)₂) can be used. Further, 2-(4-biphenylyl)-5-(4-tert-buthylphenyl)-1,3,4-oxadiazole (PBD), 1,3-bis[5-(p-tert-buthylphenyl)-1,3,4-oxadiazole-2-yl]benzene (OXD-7), 3-(4-tert-buthylphenyl)-4-phenyl-5-(4-biphenylyl)-1,2,4-triazole (TAZ), 3-(4-tert-buthylphenyl)-4-(4-ethylpheyl)-5-(4-biphenylyl)-1,2,4-triazole (p-EtTAZ), bathophenanthroline (BPhen), bathocuproin (BCP), and the like can be used in addition to such metal complexes.

As an electron injecting material for the electron injecting layer, the electron transporting material mentioned above can be used. In addition, an ultrathin film of an insulator, for example, an alkali metal halide such as LiF or CsF, a alkali earth metal halide such as CaF₂, an alkali metal oxide such as Li₂O, or the like is often used. Alkali metal complexes such as lithium acetylacetonate (Li(acac)) and 8-quinolinolato-lithium (Liq) are also effective. Furthermore, a layer in which the electron transporting material mentioned above and a metal having a low work function such as Mg, Li, and Cs are mixed, can used as the electron injecting layer. In addition, a metal oxide such as molybdenum oxide (MoOₓ), vanadium oxide (VOₓ), ruthenium oxide (RuOₓ), or tungsten oxide (WOₓ), or one of a benzoxazole derivative and one or more of alkali metal, alkali earth metal, and transition metal may be included. Titanium oxide also may be used.

The carbazole derivative according to the present invention has a high HOMO level. Thus, a hole injecting barrier from the anode formed by a material having a high work function is small, and holes are easy to inject. Therefore, by including the carbazole derivative according to the present invention in a layer being in contact with the anode, the driving voltage can be reduced.

In addition, the carbazole derivative according to the present invention also has a high LUMO level. Thus, the electron injection barrier is high, and it is thus possible to inhibit the intrusion of electrons into the anode side. Accordingly, the probability of recombination of carriers is increased, and the luminous efficiency is thus improved. Namely, when the probability of recombination of carriers is increased, less current is needed to obtain a certain luminance.

Further, when lower voltage driving and lower current driving can be achieved, the advantage that the light-emitting element has longer lifetime and higher reliability, can also be obtained.

A carbazole derivative of the present invention has a high glass transition temperature, and a favorable amorphous film can be kept at a high temperature. Thus, a film having high heat resistance can be obtained. Accordingly, a light-emitting element having a high heat resistance can be provided by using a carbazole derivative of the present invention for the light-emitting element.

Moreover, a carbazole derivative of the present invention is extremely stable for an oxidation reaction. Thus, a highly reliable light-emitting element can be obtained by using a carbazole derivative of the present invention for the light-emitting element.

### Embodiment Mode 3

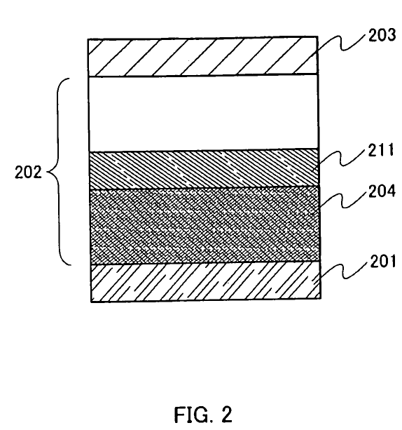

Embodiment Mode 3 will describe a case that a carbazole derivative of the present invention can be used as a hole transporting layer with reference to FIG. 2, which is different from Embodiment Mode 2.

In the light-emitting element shown in FIG. 2, a layer including a luminescent substance 202 is interposed between a first electrode 201 and a second electrode 203. In this embodiment mode, the first electrode 201 serves as an anode and the second electrode 203 serves as a cathode. A layer 204 which is closer to the anode than a light-emitting layer 211 includes a layer containing a carbazole derivative of the present invention. The layer 204 and the light-emitting layer 211 are included in the layer including a luminescent substance 202. The layer containing a carbazole derivative according to the present invention serves as a hole transporting layer.

As shown by the structure in FIG. 2, in the case that a carbazole derivative of the present invention is used as the hole transporting layer, a known material can be used as a hole injecting material for forming a hole injecting layer. Specifically, in a case of using an organic compound, a porphyrin based compound is effective, phthalocyanine (H₂-PC), copper phthalocyanine (CuPc) or the like, can be used. In addition, a chemically-doped conductive polymer compound can be used, for example, polyethylene dioxythiophene (PEDOT) which is doped with polystyrene sulfonic acid (PSS), polyaniline (PAni), or the like, can be used. In addition, inorganic semiconductive layers such as VOₓ or MoOₓ, or an ultra thin film of an inorganic insulator such as Al₂O₃ can also be employed.

It should be noted that the layer containing a carbazole derivative of the present invention is acceptable as long as the layer containing the carbazole derivative is included in the layer 204 which is closer the anode than the light-emitting layer 211, and thus, the layer containing the carbazole derivative may be in contact with the light-emitting layer 211 or not. In addition, the layer containing a carbazole derivative of the present invention may be provided so that the layer is not in contact with the first electrode 201, or the layer containing a carbazole derivative of the present invention may be formed to be in contact with the first electrode 201, and the layer may have functions of a hole injecting layer and a hole transporting layer.

A carbazole derivative of the present invention is superior in a hole transporting property, and thus, driving voltage of a light-emitting element can be reduced by using the carbazole derivative as a hole transporting layer.

In addition, the carbazole derivative according to the present invention also has a high LUMO level. Thus, the electron injection barrier is high, and it is thus possible to inhibit the intrusion of electrons into the anode side. Accordingly, the probability of recombination of carriers is increased, and the luminous efficiency is thus improved. Namely, since the probability of recombination of carriers is increased, less current is needed to obtain a certain luminance.

A carbazole derivative of the present invention has a high glass transition temperature, and a favorable amorphous film can be kept at a high temperature. Thus, a film having high heat resistance can be obtained. Accordingly, a light-emitting element having a high heat resistance can be provided by using a carbazole derivative of the present invention for the light-emitting element.

Moreover, a carbazole derivative of the present invention is extremely stable for an oxidation reaction. Thus, a highly reliable light-emitting element can be obtained by using a carbazole derivative of the present invention for the light-emitting element.

### Embodiment Mode 4

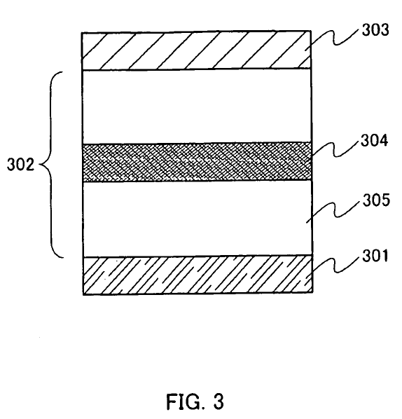

Embodiment Mode 4 will describe a case that a carbazole derivative of the present invention is used for a light-emitting layer with reference to FIG. 3.

In the light-emitting element shown in FIG. 3, a layer including a luminescent substance 302 is interposed between a first electrode 301 and a second electrode 303. In this embodiment mode, the first electrode 301 serves as an anode and the second electrode 303 serves as a cathode. A light-emitting layer 304 included in the layer including a luminescent substance 302, includes a layer containing a carbazole derivative of the present invention. The carbazole derivative of the present invention has an excellent hole transporting property, and thus can be used as a host material of the light-emitting layer. Moreover, a carbazole derivative of the present invention emits blue light or the like, it can be used as a light-emitting material.

Further, when a carbazole derivative of the present invention is used as a host material of the light-emitting layer, the layer including a carbazole derivative of the present invention may be made to also serve as a hole transporting layer. In addition, a structure is also possible, in which the layer 305 sandwiched by the first electrode 301 and the light-emitting layer 304 includes a carbazole derivative of the present invention.

A carbazole derivative of the present invention is superior in a hole transporting property, and thus, driving voltage of a light-emitting element can be reduced by using the carbazole derivative as a host material of the light-emitting layer.

In addition, the carbazole derivative of the present invention also has a high LUMO level. Thus, the electron injection barrier is high, and it is thus possible to inhibit the intrusion of electrons into the anode side. Accordingly, the probability of recombination of carriers is increased, and the luminous efficiency is thus improved. Namely, since the probability of recombination of the carriers is increased, less current is needed to obtain a certain luminance.

A carbazole derivative of the present invention has a high glass transition temperature, and a favorable amorphous film can be kept at a high temperature. Thus, a film having high heat resistance can be obtained. Accordingly, a light-emitting element having a high heat resistance can be provided by using a carbazole derivative of the present invention for the light-emitting element.

Moreover, a carbazole derivative of the present invention is extremely stable for an oxidation reaction. Thus, a highly reliable light-emitting element can be obtained by using a carbazole derivative of the present invention for the light-emitting element.

### Embodiment Mode 5

Embodiment Mode 5 will describe a light-emitting device which has the light-emitting element using the carbazole derivative according to the present invention.

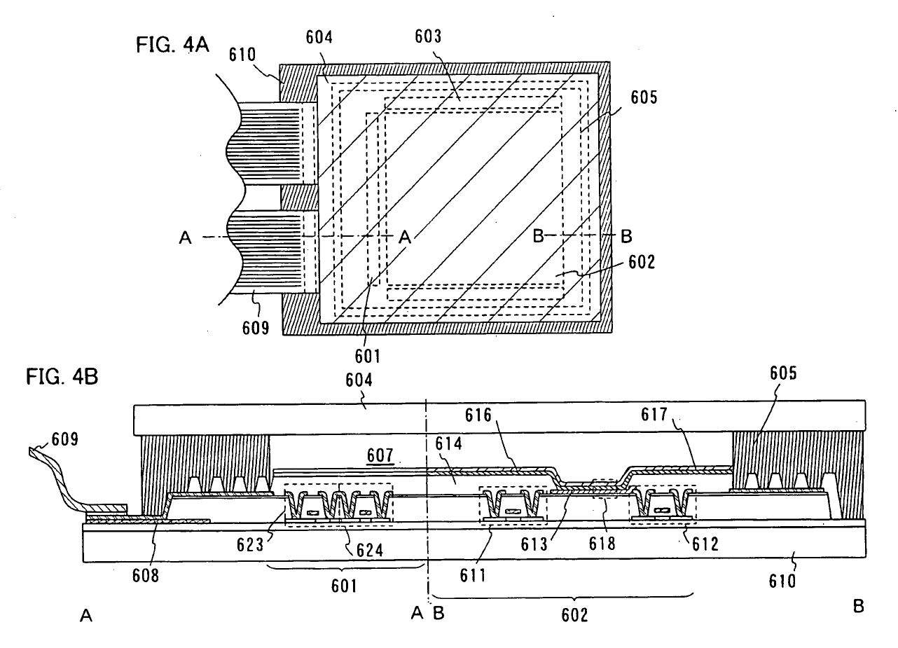

In this embodiment mode, a light-emitting device which has a light-emitting element according to the present invention in a pixel portion will be described with a reference to FIGS. 4A and 4B. FIG. 4A is a top view of the light-emitting device, and FIG. 4B is a cross sectional view along A-A′ and B-B′ in FIG. 4A. A portion 601 surrounded by a dotted line is a driver circuit portion (source side driver circuit), a portion 602 surrounded by another dotted line is a pixel portion, and a portion 603 surrounded by another dotted line is a driver circuit portion (gate side driver circuit). In addition, reference numeral 604 denotes a sealing substrate and reference numeral 605 denotes a sealing material. The inside surrounded by the sealing material 605 is an interspace 607.

A leading wiring 608 is a wiring for transmitting signals to be input to the source side driver circuit 601 and the gate side driver circuit 603, and receives signals such as a video signal, a clock signal, a start signal, and a reset signal from a FPC (Flexible Printed Circuit) 609 as an external input terminal. Although only the FPC is shown here, a printed wiring board (PWB) may be attached to the FPC. The category of the light-emitting device in the present specification includes not only light-emitting devices themselves but also light-emitting devices to which an FPC or a PWB is attached.

Next, a sectional structure of the light-emitting device will be described with reference to FIG. 4B. The driver circuit portion and the pixel portion are formed over an element substrate 610. In FIG. 4B, the source side driver circuit 601 of the driver circuit portions and one pixel in the pixel portion 602 are shown.

In the source side driver circuit 601, a CMOS circuit in which an n-channel TFT 623 and a p-channel TFT 624 are combined, is formed. The driver circuit constituted by TFTs may be formed with a known CMOS circuit, PMOS circuit, or NMOS circuit. Although this embodiment mode describes the case that driver circuits are formed over the same substrate, the driver circuit are not necessarily formed over the same substrate, and the driver circuit can be formed outside the substrate.

The pixel portion 602 includes plural pixels. Each of the pixels includes a switching TFT 611, a current controlling TFT 612, and a first electrode 613 electrically connected to a drain of the current controlling TFT 612. An insulator 614 is formed to cover an end portion of the first electrode 613. Here, a positive photosensitive acrylic resin film is used to form the insulator 614.

In addition, an upper or lower end portion of the insulator 614 is made to have a curved surface with a curvature in order to improve the coverage. For example, in the case of using positive photosensitive acrylic as a material of the insulator 614, it is preferable that only the upper end portion of the insulator 614 be made to have a curved surface with a curvature radius (0.2 μm to 3 μm). Besides, as the insulator 614, it is possible to use a negative photosensitive material which is insoluble in an etchant by light and a positive photosensitive material which is soluble in an etchant by light.

On the first electrode 613, a layer including a luminescent substance 616 and a second electrode 617 are formed. Here, it is preferable to use a material having a high work function as a material to be used for the first electrode 613 which functions as an anode. For example, it is possible to use stacked structures such as a stacked layer of a titanium nitride film and a film mainly containing aluminum, and a three-layer structure of a titanium nitride film, a film mainly containing aluminum, and a titanium nitride film, and the like, in addition to a single layer such as an ITO film, an indium tin oxide film containing silicon, an indium oxide film containing zinc oxide of 2% to 20%, a titanium nitride film, a chromium film, a tungsten film, a Zn film, or a Pt film. When a stacked structure is employed, it has a low resistance as a wiring, favorable ohmic contact can be made, and the electrode can function as an anode.

The layer including a luminescent substance 616 is formed by a known method such as an evaporation method using an evaporation mask, an inkjet method, and a spin coating method. The layer including a luminescent substance 616 contains a carbazole derivative of the present invention. As a material used by being combined with the carbazole derivative according to the present invention, a low molecular weight material, an intermediate molecular weight material (including an oligomer and an dendrimer), or a high molecular weight material may be used. In addition, as a material used for the layer including a luminescent substance, in general, an organic compound is often used as a single layer or a stacked layer. However, the present invention includes a structure in which an inorganic compound is used for a part of a film including an organic compound.

The carbazole derivative according to the present invention has an excellent hole injecting property, and it is preferably used as a hole injecting material. In addition, the carbazole derivative of the present invention has also an excellent hole transporting property, and can be used as a hole transporting material.

As a material used for the second electrode (cathode) 617 formed on the layer including a luminescent substance 616, it is preferable to use a material having a low work function (e.g., Al, Mg, Li, Ca, an alloy or a compound thereof such as MgAg, MgIn, AlLi, CaF₂, LiF, and calcium nitride). When light generated in the layer including a luminescent substance 616 passes through the second electrode 617, a stacked layer of a thin metal film with a thin thickness and a transparent conductive film (such as ITO, indium oxide containing zinc oxide of 2% to 20%, indium tin oxide containing silicon, or zinc oxide (ZnO)) may be used as the second electrode (cathode) 617.

The sealing substrate 604 and the element substrate 610 are bonded to each other with the sealing material 605, and thus, a structure can be obtained, in which a light-emitting element 618 is provided in the interspace 607 surrounded by the element substrate 610, the sealing substrate 604, and the sealing material 605. The interspace 607 is filled with filler. There is a case in that the sealing material 605 fills the interspace 607, in addition to a case in that an inert gas (such as nitrogen or argon) fills the interspace 607.

It is to be noted that it is preferable to use an epoxy resin for the sealing material 605. A material which hardly transmits water and oxygen is preferable. Further, as a material used for the sealing substrate 604, a plastic substrate including a material such as FRP (Fiberglass-Reinforced Plastics), PVF (polyvinyl fluoride), Mylar®, polyester, or acrylic can be used as well as a glass substrate and a quartz substrate.

As mentioned above, the light-emitting device which has the light-emitting element according to the present invention can be obtained.

The light-emitting device of the present invention has a light-element including the carbazole derivative which has excellent hole injecting and hole transporting properties. Therefore, low-voltage driving and low-current driving of a light-emitting element are possible and thus, it is possible to make the light-emitting device to have a longer life time and higher reliability.

Since the lower voltage driving and the lower current driving of the light-emitting device are possible, lower power consumption can also be realized.

A carbazole derivative of the present invention has a high glass transition temperature, and a favorable amorphous film can be kept at a high temperature. Thus, a film having high heat resistance can be obtained. Moreover, a carbazole derivative of the present invention is extremely stable for an oxidation reaction. Thus, a highly reliable light-emitting device can be obtained by using a carbazole derivative of the present invention for the light-emitting device.

### Embodiment Mode 6

Embodiment Mode 6 will describe various electronic devices each including a light-emitting device formed using a light-emitting element of the present invention as a part thereof.

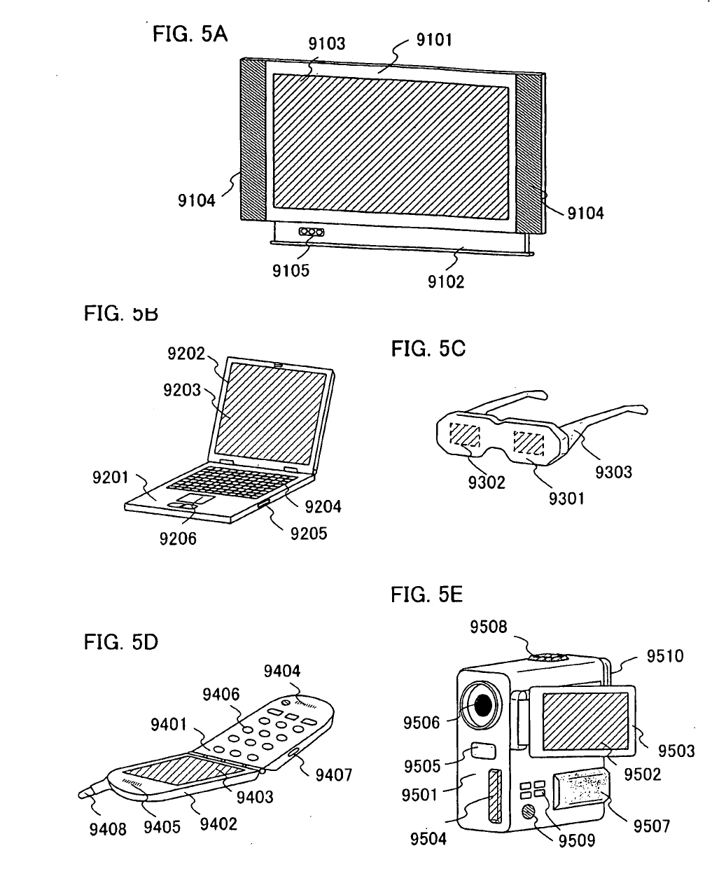

Electronic devices manufactured using a light-emitting device having a light-emitting element of the present invention are, for example, cameras such as video cameras or digital cameras, goggle type displays, navigation systems, sound reproduction devices (such car audios or audio components), computers, game machines, portable information terminals (such as mobile computers, cell phones, portable game machines, or electronic books), image reproduction devices utilizing a recording medium (such as devices which can reproduce a recording medium such as a digital versatile disk (DVD) and is equipped with a display device capable of displaying the image) and the like. These electronic devices are specifically shown in FIGS. 5A to 5E.

FIG. 5A shows a television receiving machine which includes a casing 9101, a supporting stand 9102, a display portion 9103, speaker portions 9104, a video input terminal 9105, and the like. The television receiving machine is manufactured by using a light-emitting device having a light-emitting element of the present invention for the display portion 9103. By using the light-emitting device of the present invention, a television receiving machine having a display portion with a long lifetime, low power consumption and high reliability, can be provided. It is to be noted that the category of the television receiving machine includes all types of information display devices, e.g., a display device for a computer, one for TV broadcast reception, one for advertisement display, and so on.

FIG. 5B shows a computer which includes a main body 9201, a casing 9202, a display portion 9203, a keyboard 9204, an external connection port 9205, a pointing mouse 9206, and the like. The computer is manufactured by using a light-emitting device having a light-emitting element of the present invention for the display portion 9203. By using the light-emitting device of the present invention, a computer having a display portion with a long lifetime, low power consumption and high reliability, can be provided.

FIG. 5C shows a goggle-type display which includes a main body 9301, display portions 9302, arm portions 9303, and the like. The goggle-type display is manufactured by using a light-emitting device having a light-emitting element of the present invention for the display portion 9302. By using the light-emitting device of the present invention, a goggle-type display having a display portion with a long lifetime, low power consumption and high reliability, can be provided.

FIG. 5D shows a cell phone which includes a main body 9401, a casing 9402, a display portion 9403, an audio input portion 9404, an audio output portion 9405, operation keys 9406, an external connection port 9407, an antenna 9408, and the like. The cell phone is manufactured by using a light-emitting device having a light-emitting element of the present invention for the display portion 9403. By using the light-emitting device of the present invention, a cell phone having a display portion with a long lifetime, low power consumption and high reliability, can be provided. In addition, the power consumption of the cell phone can be suppressed by displaying white characters against black in the display portion 9403.

FIG. 5E shows a camera which includes a main body 9501, a display portion 9502, a casing 9503, an external connection port 9504, a remote control receiving portion 9505, an image receiving portion 9506, a battery 9507, an audio input portion 9508, operation keys 9509, an eyepiece portion 9510, and the like. The camera is manufactured by using a light-emitting device having a light-emitting element of the present invention for the display portion 9502. By using the light-emitting device of the present invention, a camera having a display portion with a long lifetime, low power consumption and high reliability, can be provided.

As thus described, a light-emitting device having a light-emitting element according to the present invention can be applied in an extremely wide range, and the light-emitting device can be applied to electronic devices of every field. By using a light-emitting device having a light-emitting element of the present invention, highly reliable electronic devices having a long lifetime with low power consumption can be provided.

### EXAMPLE 1

A synthesis method of 3-[N-(9-phenylcarbazole-3-yl)-N-phenylamino]-9-phenylcarbazole (PCzPCA1) which is represented by the structural formula (12) will be described as one example of a carbazole derivative of the present invention.

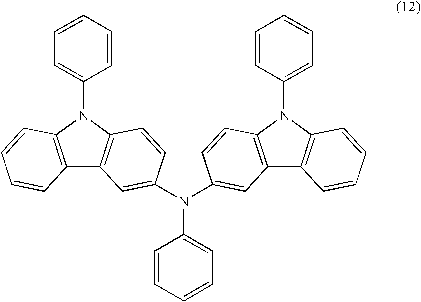

[Step 1] A synthesis method of 3-bromo-9-phenylcarbazole is described. A synthesis scheme of 3-bromo-9-phenylcarbazole is shown by (A-3).

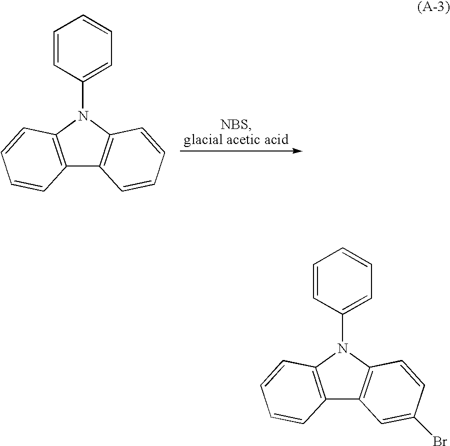

Firstly, 24.3 g (100 mmol) of 9-phenylcarbazole was dissolved in 600 ml of glacial acetic acid, and 17.8 g (100 mmol) of N-bromosuccinimide was slowly added thereto. The mixture was stirred overnight at a room temperature. This glacial acetic acid solution was dropped in 1 L of ice water while stirring it. A precipitated white solid was washed three times with water. This solid was dissolved in 150 ml of diethyl ether, and washed with a saturated sodium hydrogencarbonate solution and water. This organic layer was dried with magnesium sulfate, and filtered. The obtained filtrate was concentrated. About 50 ml of methanol was added into the thus obtained concentrated solution and uniformly dissolved therein. This solution was left still to precipitate a white solid. This solid was collected and dried to obtain 28.4 g (the yield: 88%) of 3-bromo-9-phenylcarbazole, which was white powder.

[Step 2] A synthesis method of 3-(N-phenylamino)-9-phenylcarbazole (PCA) will be described. A synthesis scheme of PCA is shown by (A-4).

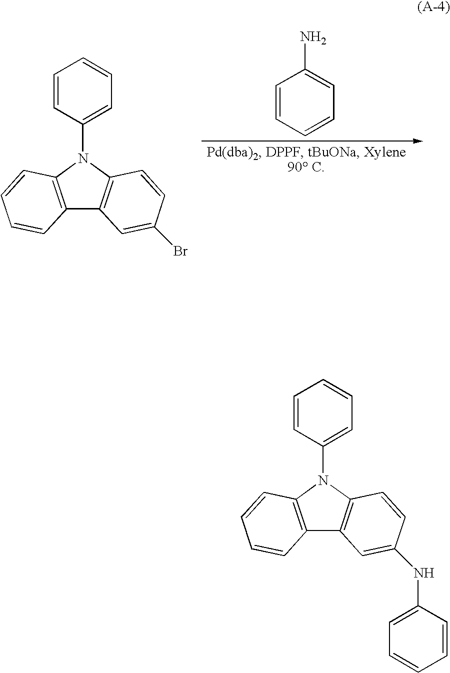

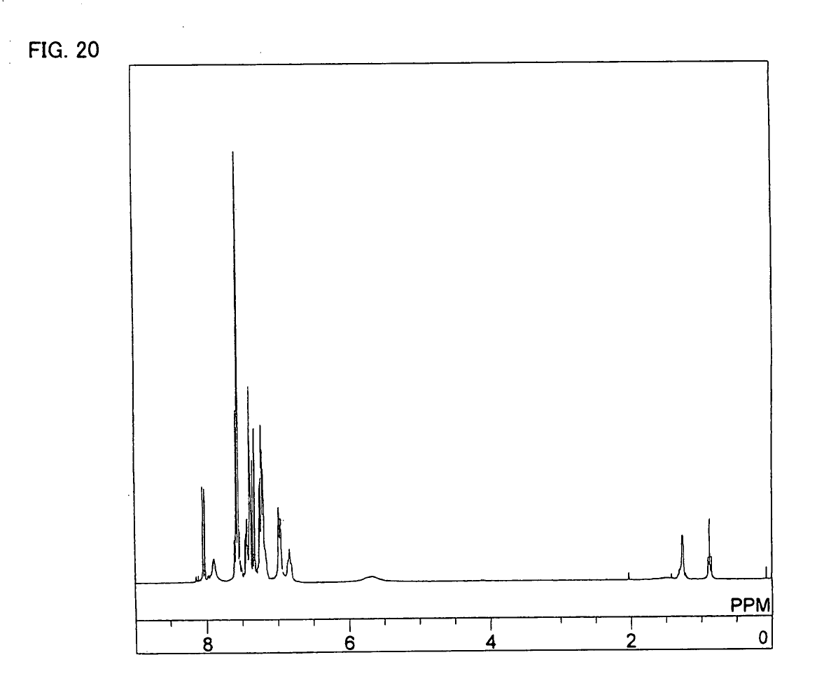

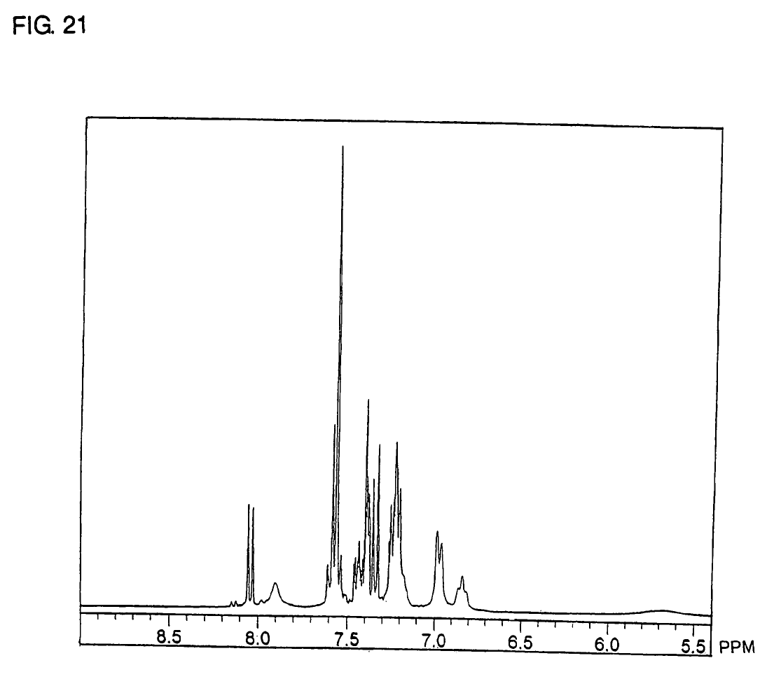

Under nitrogen, 110 ml of dehydrated xylene and 7.0 g (75 mmol) of aniline were added to a mixture of 19 g (60 mmol) of 3-bromo-9-phenylcarbazole, 340 mg (0.6 mmol) of bis(dibenzylideneacetone)palladium (0), 1.6 g (3.0 mmol) of 1,1-bis (diphenylphosphino) ferrocene, and 13 g (180 mmol) of sodium-tert-butoxide. This mixture was stirred while heating under the nitrogen atmosphere at 90° C. for 7.5 hours. After the termination of the reaction, about 500 ml of heated toluene was added to the suspension and this suspension was filtered through florisil, alumina and Celite®. The thus obtained filtrate was concentrated and hexane-ethyl acetate was added to the concentrated solution and it was irradiated with an ultrasonic wave. The thus obtained suspension was filtered and the residue was dried to obtain 15 g (the yield: 75%) of 3-(N-phenylamino)-9-phenylcarbazole which was cream-colored powder. NMR data are shown below. ¹H-NMR (300 MHz, CDCl₃): δ=5.69 (s, 1H), 6.84 (t, J=6.9, 2H), 6.97 (d, J=7.8, 2H), 7.20-7.61 (m, 13H), 7.90 (s, 1H), 8.04 (d, J=7.8, 1H). FIG. 20 shows a chart of ¹H-NMR, and FIG. 21 shows an enlarged view of the portion of 5.5 to 9.0 ppm in FIG. 20.

[Step 3] A synthesis method of 3-iodo-9-phenylcarbazole is described. A synthesis scheme of 3-iodo-9-phenylcarbazole is shown by (A-5).

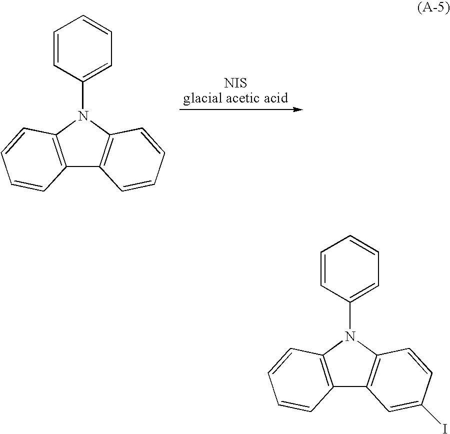

24.3 g (100 mmol) of 9-phenylcarbazole was dissolved in 600 ml of glacial acetic acid, 22.5 g (100 mmol) of N-iodosuccinimide was slowly added thereto, and then stirring was carried out at a room temperature from overnight. The generated precipitation was filtered and the residue was washed by a saturated sodium hydrogencarbonate water solution, water, and methanol, then was dried. 24.7 g (yield 67%) of 3-iodo-9-phenylcarbazole which was white powder, was obtained.

A synthesis of 3-iodo-9-phenylcarbazole can also be conducted with the following method. A synthesis scheme of 3-iodo-9-phenylcarbazole is shown by (A-5b).

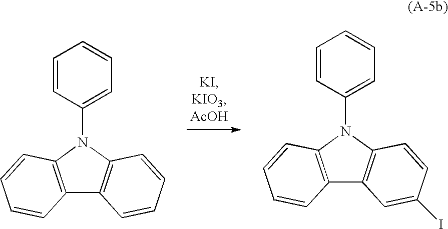

10 g (10.0 mmol) of 9-phenyl carbazole, 838 mg (5.0 mmol) of potassium iodide, 1.1 g (5.0 mmol) of potassium iodate, and 30 mL of glacial acetic acid were put in a three-neck flask and refluxed for 1 hour at 120° C. After the reaction, the reaction solution was cooled sufficiently and added into water, extracted with toluene, an organic layer was washed with saturated sodium chloride solution once, was dried with magnesium sulfate. This solution was filtered naturally, and the obtained filtrate was concentrated. Then, it was recrystallized with acetone and methanol. 8.0 g (the yield: 50%) of a white solid, which was an objective substance, was obtained.

By employing a synthesis method shown by a synthesis scheme (A-5b), 3-iodo-9-phenyl carbazole can be synthesized using a more inexpensive material, thereby reducing costs.

### [Step 4]

A synthesis method of 3-[N-(9-phenylcarbazole-3-yl)-N-phenyl amino]-9-phenylcarbazole (PCzPCA1) is described. A synthesis scheme of PCzPCA1 is shown by (A-6).

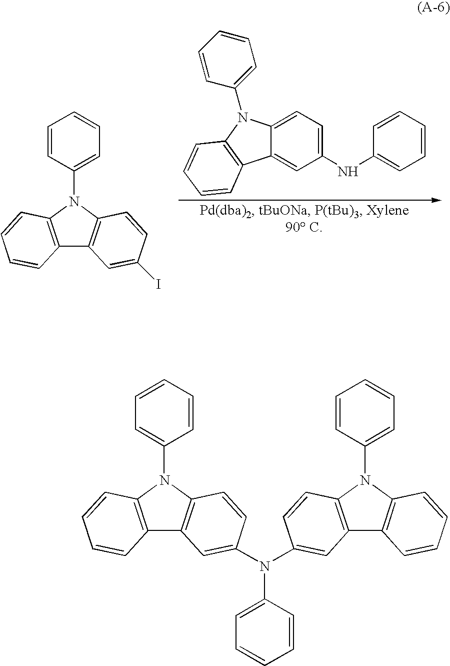

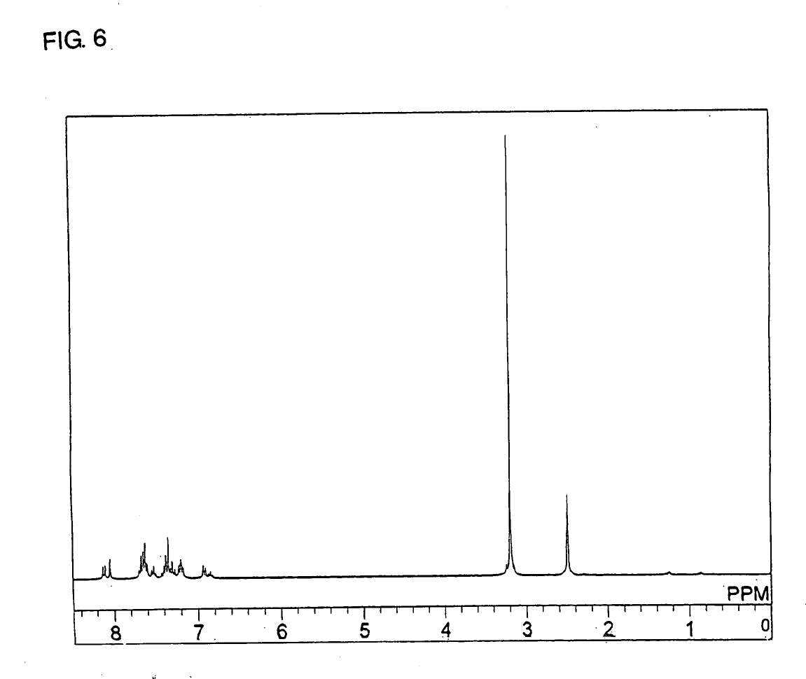

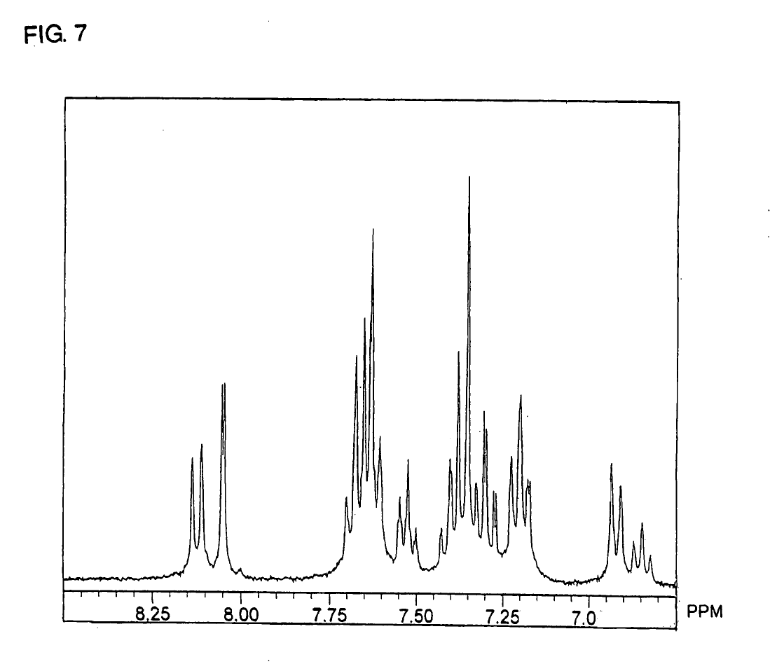

Under nitrogen, 40 ml of dehydrated xylene was added to a mixture of 3.7 g (10 mmol) of 3-iodo-9-phenylcarbazole, 3.4 g (10 mmol) of PCA, 57 mg (0.1 mmol) of bis(dibenzylideneacetone)palladium (0), 0.2 mL (0.5 mmol) of tri-tert-butylphosphine 49 wt % hexane solution, and 3.0 g (30 mmol) of sodium-tert-butoxide. This mixture was stirred while heating under nitrogen atmosphere at 90° C. for 6.5 hours. After the termination of the reaction, about 500 ml of heated toluene was added to the suspension and this suspension was filtered through florisil, alumina and Celite®. The thus obtained filtrate was concentrated and the concentrated solution was purified by using silica gel column chromatography (toluene:hexane=1:1). This was concentrated and hexane-ethyl acetate was added thereinto to conduct recrystallization. 3.2 g (the yield: 56%) of 3-[N-(9-phenylcarbazole-3-yl)-N-phenylamino]-9-phenylcarbazole which was cream-colored powder, was obtained. NMR data are shown below. ¹H-NMR (300 MHz, DMSO-d): δ=6.85 (t, J=7.5, 1H), 6.92 (d, J=7.8, 2H), 7.17-7.70 (m, 22H), 8.05 (d, J=2.1, 2H), 8.12 (d, J=7.8, 2H). FIG. 6 shows a chart of ¹H-NMR, and FIG. 7 shows an enlarged view of the portion of 6.75 to 8.50 ppm in FIG. 6.

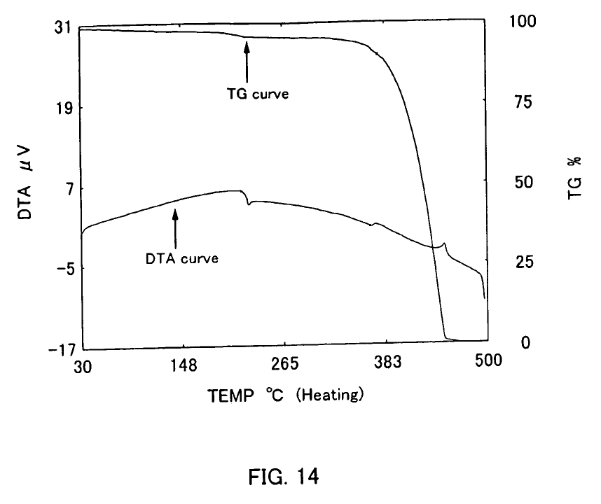

The thermogravimetry-differential thermal analysis (TG-DTA) of the thus obtained PCzPCA1 was performed. The results are shown in FIG. 14. In FIG. 14, the vertical axis on the left side indicates the heat quantity (μV) and the vertical axis on the right side indicates the gravity (%; the gravity at the start of measurement is expressed as 100%). Furthermore, the lower horizontal axis represents a temperature (° C.). By utilizing a thermo-gravimetric/differential thermal analyzer (TG/DTA 320, Seiko Instruments Inc.), thermo-physical properties were measured at the temperature rising velocity of 10° C./min under a nitrogen atmosphere. As a result, based on the gravity-temperature relation (thermogravimetric measurement), the temperature at which the gravity becomes 95% or less of the gravity at the start of the measurement, was 375° C. under normal pressure.

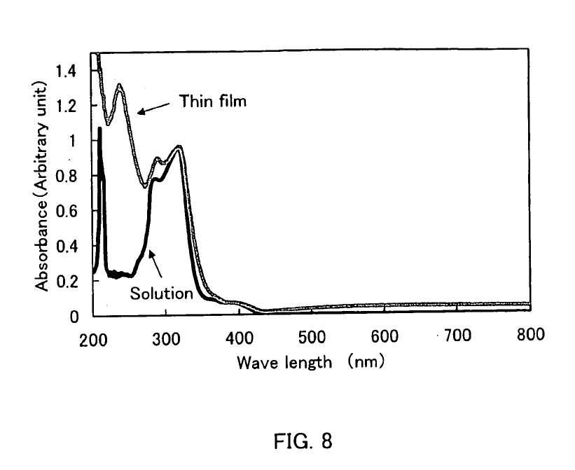

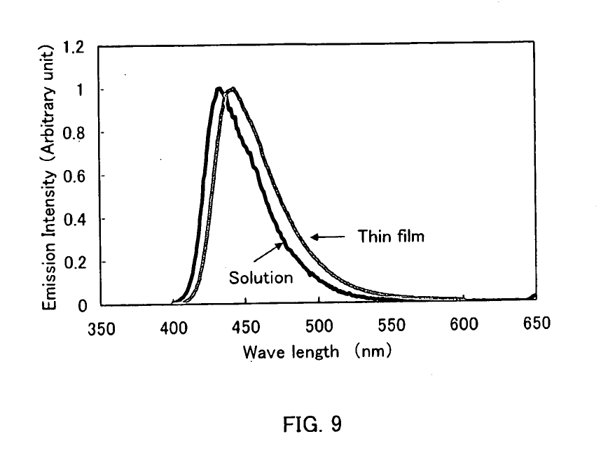

Absorption spectra of the toluene solution of PCzPCA1 and a thin film of PCzPCA1 are shown in FIG. 8. An ultraviolet-visible spectrophotometer (manufactured by JASCO Corporation, V-550) was used for the measurement. The solution was put in a quartz cell and a thin film was deposited on a quartz substrate as samples, and absorption spectra of them, from which an absorption spectrum of quartz was taken, were shown in FIG. 8. In FIG. 8, the horizontal axis indicates wavelength (nm) and the vertical axis indicates absorbance (arbitrary unit). The maximum absorption wavelength was 320 nm in the case of the toluene solution, and 321 nm in the case of the thin film. Emission spectra of the toluene solution of PCzPCA1 and the thin film of PCzPCA1 are shown in FIG. 9. In FIG. 9, the horizontal axis indicates wavelength (nm) and the vertical axis indicates emission intensity (arbitrary unit). The maximum emission wavelength was 435 nm (excitation wavelength 325 nm) in the case of the toluene solution, and 443 nm (excitation wavelength 380 nm) in the case of the thin film.

Further, the HOMO level and LUMO level of PCzPCA1 in a state of a thin film were measured. A value of the HOMO level was obtained by converting a value of ionization potential measured by a photoelectron spectrometer (manufactured by Riken Keiki Co., Ltd., AC-2) into a negative value. A value of the LUMO level was obtained by using an edge of the absorption spectrum of the thin film in FIG. 8 as an energy gap and adding the value of the absorption edge to the value of the HOMO level. As a result, the HOMO level and the LUMO level were −5.17 eV and −1.82 eV, respectively.

In addition, an oxidation reaction characteristic of PCzPCA1 was measured by a cyclic voltammetry (CV) measurement. Further, an electrochemical analyzer (ALS model 600A, BAS Inc.) was used for the measurement.

As for a solution used in the CV measurement, dehydrated dimethylformamide (DMF) was used as a solvent. Tetraperchlorate-n-butylammonium (n-Bu₄NClO₄), which was a supporting electrolyte, was dissolved in the solvent such that the concentration of the tetraperchlorate-n-butylammonium was 100 mmol/L. Also, the PCzPCA1, which was an object to be measured, was dissolved therein such that the concentration thereof was set to be 1 mmol/L. Further, a platinum electrode (a PTE platinum electrode, BAS Inc.) was used as a work electrode. A platinum electrode (a VC-3 Pt counter electrode (5 cm), BAS Inc.) was used as an auxiliary electrode. An Ag/Ag⁺ electrode (an RE 5 nonaqueous reference electrode, BAS Inc.) was used as a reference electrode.

The oxidation reaction characteristic was measured as follows. After a potential of the work electrode with respect to the reference electrode was changed to 0.5 V from −0.16 V, a scan for changing the potential to −0.16 V from 0.5 V was set as one cycle, and 100 cycle measurements were carried out. Further, the scanning speed of the CV measurement was set to be 0.1 V/s.

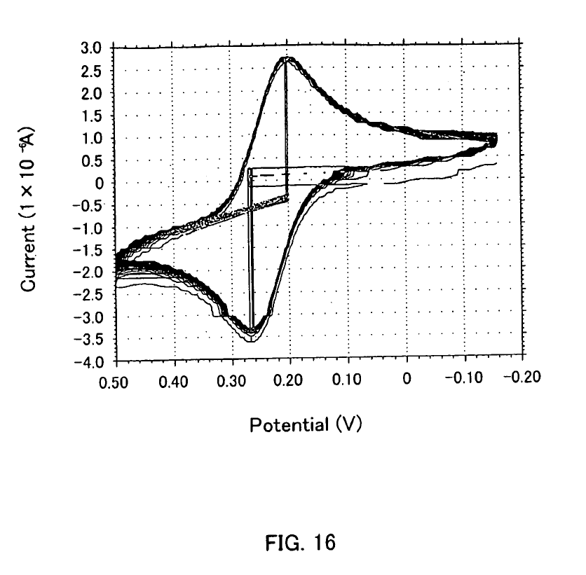

Results of measuring the oxidation reaction characteristic of PCzPCA1 are shown in FIG. 16. In FIG. 16, the horizontal axis represents a potential (V) of the work electrode with respect to the reference electrode, while the vertical axis represents current flowing between the work electrode and the auxiliary electrode (1×10⁻⁶ A). According to FIG. 16, it was known that an oxidation potential was 0.27 V (vs. Ag/Ag⁺ electrode). Although the scan was repeated 100 times, the peak position and the peak intensity of the CV curve were hardly changed in the oxidation reaction. Thus, it was known that the carbazole derivative of the present invention, was absolutely stable for the oxidation reaction.

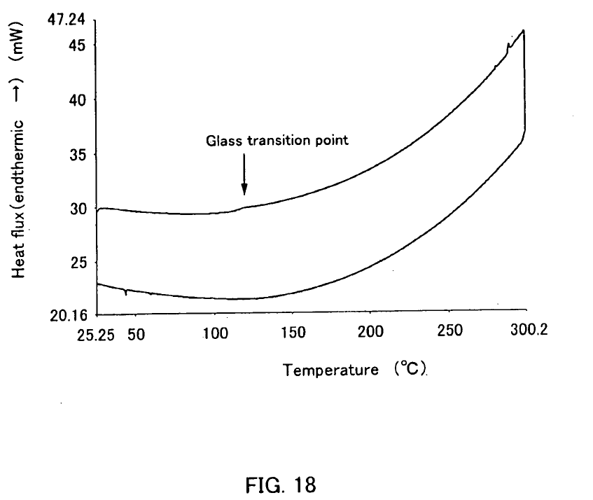

The glass transition temperature of the obtained compound PCzPCA1 was examined with a differential scanning calorimeter (Pyris 1 DSC manufactured by Perkin Elmer Co., Ltd.) Measurement results by DSC are shown in FIG. 18. According to the measurement results, it was found that the glass transition temperature of the obtained compound was 112° C. As just described, the obtained compound exhibits as high as 120° C., and has excellent heat resistance. In addition, the crystallization peak of the obtained compound is not shown in FIG. 18, and thus, it can be found that the obtained compound is difficult to be crystallized.

### EXAMPLE 2

A synthesis method of 3,6-bis[N-(9-phenylcarbazole-3-yl)-N-phenylamino]-9-phenylcarbazole (PCzPCA2), which is represented by the structural formula (38), will be described as one example of a carbazole derivative of the present invention.

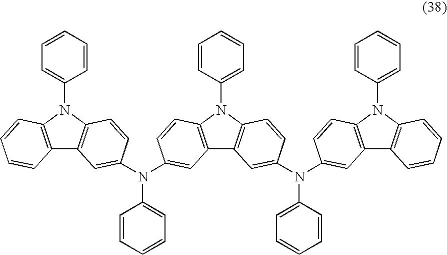

[Step 1] A synthesis method of 3,6-diiodo-9-phenylcarbazole is described. A synthesis scheme of 3,6-diiodo-9-phenylcarbazole is shown by (A-7).

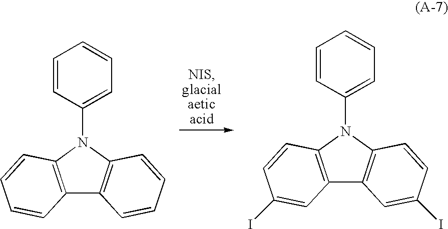

Firstly, 24.3 g (100 mmol) of 9-phenylcarbazole was dissolved in 700 ml of glacial acetic acid, and 44.9 g (200 mmol) of N-iodosuccinimide was slowly added thereto. The mixture was stirred overnight at a room temperature. The generated precipitation was filtered and the residue was washed by a saturated sodium hydrogencarbonate water solution, water, and methanol, then was dried. 47.0 g (yield 95%) of 3,6-diiodo-9-phenyl carbazole which was white powder, was obtained.

[Step 2] A synthesis method of 3,6-bis[N-(9-phenylcarbazole-3-yl)-N-phenylamino]-9-phenylcarbazole (PCzPCA2) will be described. A synthesis scheme of PCzPCA2 is shown by (A-8).

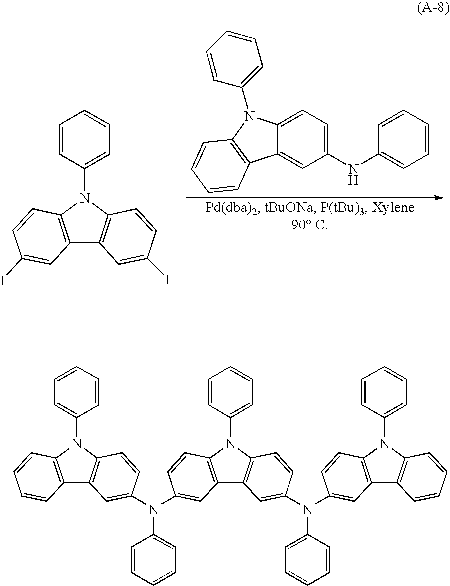

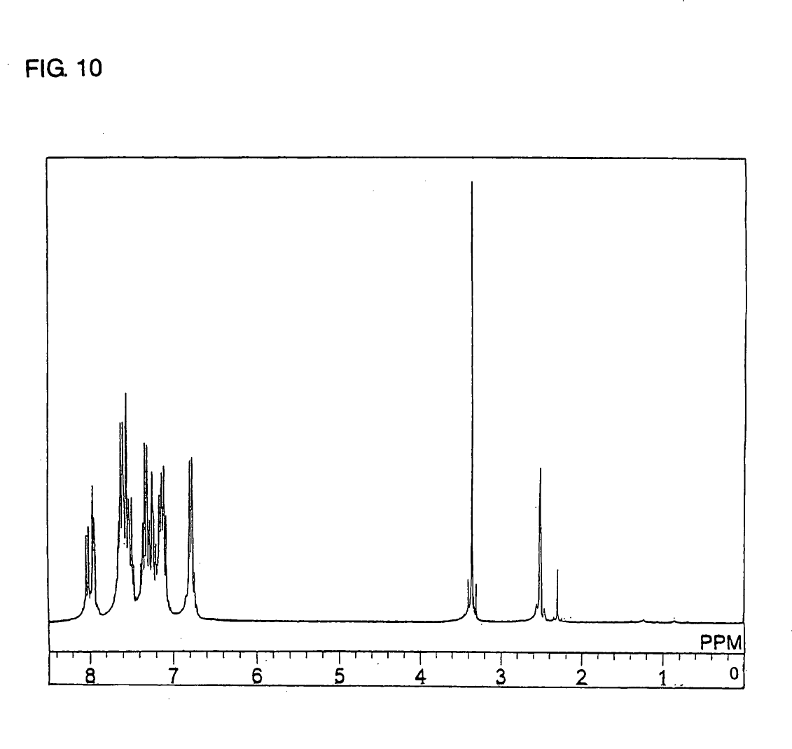

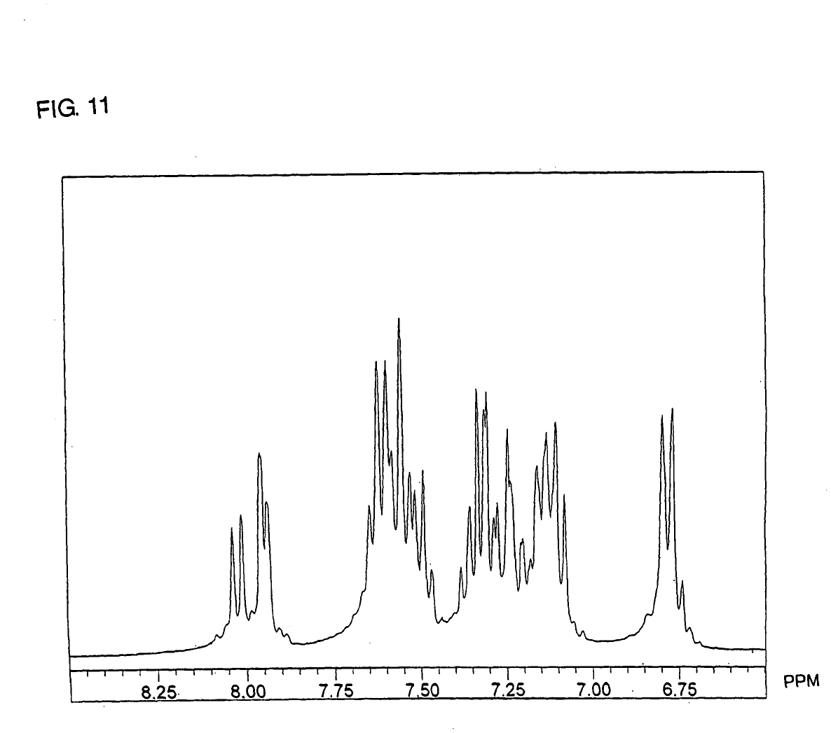

Under nitrogen, 30 ml of dehydrated xylene was added to a mixture of 2.5 mg (5 mmol) of 3,6-diiodo-9-phenylcarbazole, 3.4 g (10 mmol) of PCA, 30 mg (0.05 mmol) of bis(dibenzylidene acetone) palladium (0), 0.2 mL of a hexane solution with 49 wt % of tri-tert-butylphosphine, and 3.0 g (30 mmol) of sodium-tert-butoxide. This mixture was stirred while heating under nitrogen atmosphere at 90° C. for 6.5 hours. After the termination of the reaction, about 500 ml of heated toluene, was added to the suspension and this suspension was filtered through florisil, alumina and Celite®. The thus obtained filtrate was concentrated and the concentrated solution was purified by using silica gel column chromatography (toluene:hexane=1:1). This was concentrated and hexane-ethyl acetate was added thereinto to conduct recrystallization. 2.5 g (the yield: 55%) of 3,6-bis[N-(9-phenylcarbazole-3-yl)-N-phenylamino]-9-phenylcarbazole which was cream-colored powder, was obtained. NMR data are shown below. ¹H-NMR (300 MHz, DMSO-d): δ=6.74-6.80 (m, 6H), 7.08-7.64 (m, 33H), 7.94-8.04 (m, 6H). FIG. 10 shows a chart of ¹H-NMR, and FIG. 11 shows an enlarged view of the portion of 6.50 to 8.50 ppm in FIG. 10.

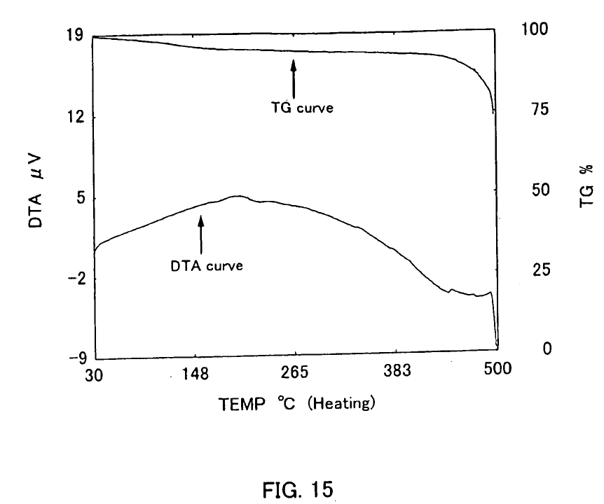

The thermogravimetry-differential thermal analysis (TG-DTA) of the thus obtained PCzPCA2 was performed. The results are shown in FIG. 15. In FIG. 15, the vertical axis on the left side indicates the heat quantity (IV) and the vertical axis on the right side indicates the gravity (%; the gravity at the start of measurement is expressed as 100%). Furthermore, the lower horizontal axis represents a temperature (° C.). By utilizing a thermo-gravimetric/differential thermal analyzer (TG/DTA 320, Seiko Instruments Inc.), thermophysical properties were measured at the temperature rising velocity of 10° C./min under nitrogen atmosphere. As a result, based on the gravity-temperature relation (thermogravimetric measurement), the temperature at which the gravity becomes 95% or less of the gravity at the start of the measurement, was 476° C. under normal pressure.

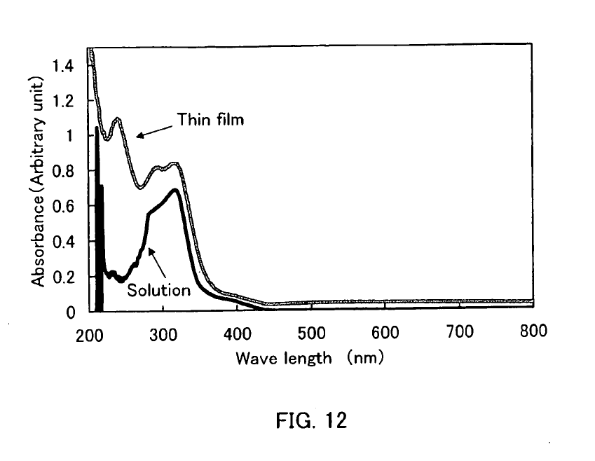

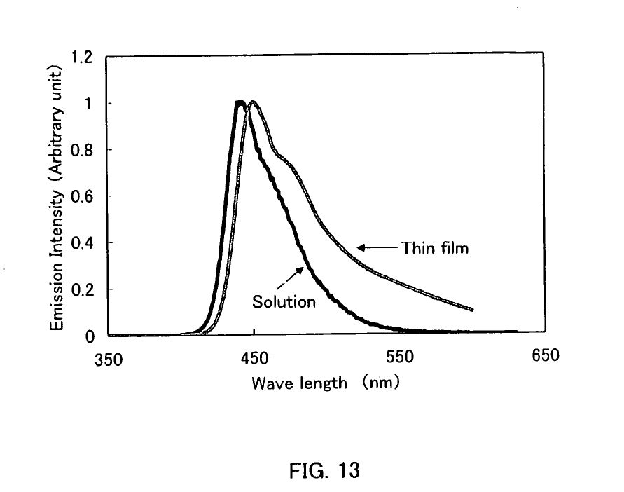

Absorption spectra of the toluene solution of PCzPCA2 and a thin film of PCzPCA2 are shown in FIG. 12. An ultraviolet-visible spectrophotometer (manufactured by JASCO Corporation, V-550) was used for the measurement. The solution was put in a quartz cell, and the thin film was deposited on a quartz substrate as samples, and absorption spectra of them, from which absorption spectrum of quartz was taken, are shown in FIG. 12. In FIG. 12, the horizontal axis indicates wavelength (nm) and the vertical axis indicates absorbance (arbitrary unit). The largest absorption wavelength was 320 nm in the case of the toluene solution, and 320 nm in the case of the thin film. Emission spectra of the toluene solution of PCzPCA2 and the thin film of PCzPCA2 are shown in FIG. 13. In FIG. 13, the horizontal axis indicates wavelength (nm) and the vertical axis indicates emission intensity (arbitrary unit). The highest emission wavelength was 442 nm (excitation wavelength 325 nm) in the case of the toluene solution, and 449 nm (excitation wavelength 320 nm) in the case of the thin film.

Further, the HOMO level and LUMO level of PCzPCA2 in a state of a thin film were measured. A value of the HOMO level was obtained by converting a value of ionization potential measured by a photoelectron spectrometer (manufactured by Riken Keiki Co., Ltd., AC-2) into a negative value. A value of the LUMO level was obtained by using an edge of the absorption spectrum of the thin film in FIG. 12 as an energy gap and adding the value of the absorption edge to the value of the HOMO level. As a result, the HOMO level and the LUMO level were −5.10 eV and −1.75 eV, respectively.

In addition, an oxidation reaction characteristic of PCzPCA2 was measured by a cyclic voltammetry (CV) measurement. Further, an electrochemical analyzer (ALS model 600A, BAS Inc.) was used for the measurement.

As for a solution used in the CV measurement, dehydrated dimethylformamide (DMF) was used as a solvent. Tetraperchlorate-n-butylammonium (n-Bu₄NClO₄), which was a supporting electrolyte, was dissolved in the solvent such that the concentration of the tetraperchlorate-n-butylammonium was 100 mmol/L. Also, the PCzPCA2, which was an object to be measured, was dissolved such that the concentration thereof was set to be 1 mmol/L. Further, a platinum electrode (a PTE platinum electrode, BAS Inc.) was used as a work electrode. A platinum electrode (a VC-3 Pt counter electrode (5 cm), BAS Inc.) was used as an auxiliary electrode. An Ag/Ag⁺ electrode (an RE 5 nonaqueous reference electrode, BAS Inc.) was used as a reference electrode.

The oxidation reaction characteristic was measured as follows. After a potential of the work electrode with respect to the reference electrode was changed to 0.33 V from −0.01 V, a scan for changing the potential to −0.01 V from 0.33 V was set as one cycle, and 100 cycle measurements were carried out. Further, the scanning speed of the CV measurement was set to be 0.1 V/s.

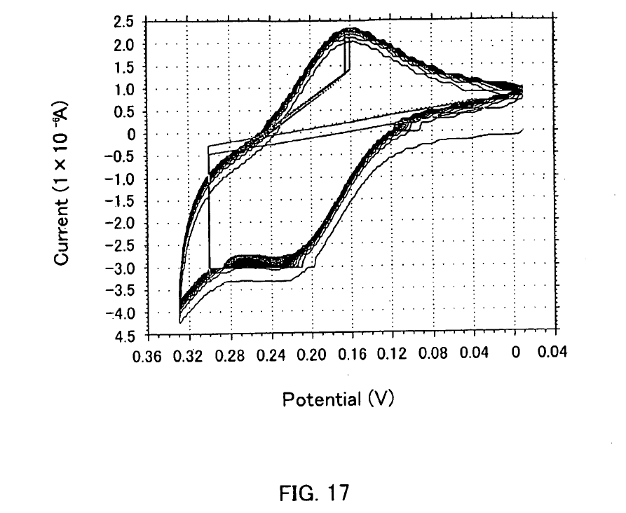

Results of measuring the oxidation reaction characteristic of the PCzPCA2 are shown in FIG. 17. In FIG. 17, the horizontal axis represents a potential (V) of the work electrode with respect to the reference electrode, while the vertical axis represents current flowing between the work electrode and the auxiliary electrode (1×10⁻⁶ A). According to FIG. 17, it was known that an oxidation potential was 0.22 V (vs. Ag/Ag⁺ electrode). Although the scan was repeated 100 times, the peak position and the peak intensity of a CV curve were hardly changed in the oxidation reaction. Thus, it was known that the carbazole derivative of the present invention, was absolutely stable for the oxidation reaction.

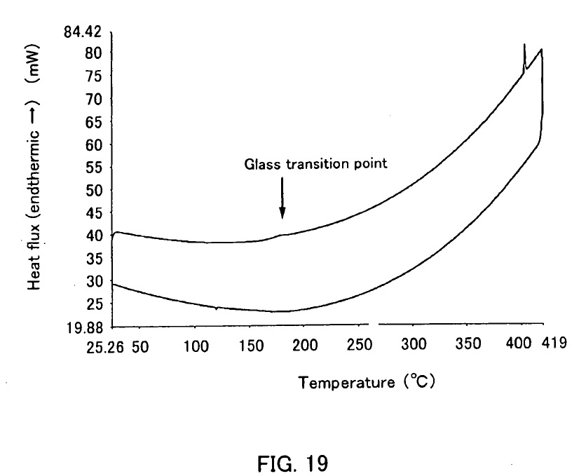

The glass transition temperature of the obtained compound PCzPCA2 was examined with a differential scanning calorimeter (Pyris 1 DSC manufactured by Perkin Elmer Co., Ltd.). Measurement results by DSC are shown in FIG. 19. According to the measurement results, it can be found that the glass transition temperature of the obtained compound was 168° C. As just described, the obtained compound exhibits a high glass transition temperature of as high as 168° C., and has excellent heat resistance. In addition, the crystallization peak of the obtained compound is not shown in FIG. 19, and thus, it can be found that the obtained compound is difficult to be crystallized.

### EXAMPLE 3

A synthesis method of 3-[N-(1-naphtyl)-N-(9-phenylcarbazole-3-yl)amino]-9-phenylcarbazole (PCzPCN1), which is represented by the structural formula (17), will be described as one example of a carbazole derivative of the present invention.

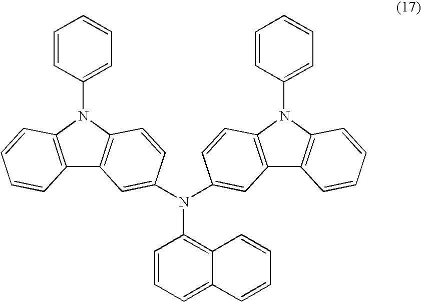

[Step 1] A synthesis method of 3-[N-(1-naphtyl)amino]-9-phenylcarbazole (PCN) is described. A synthesis scheme of PCN is shown by (A-9).

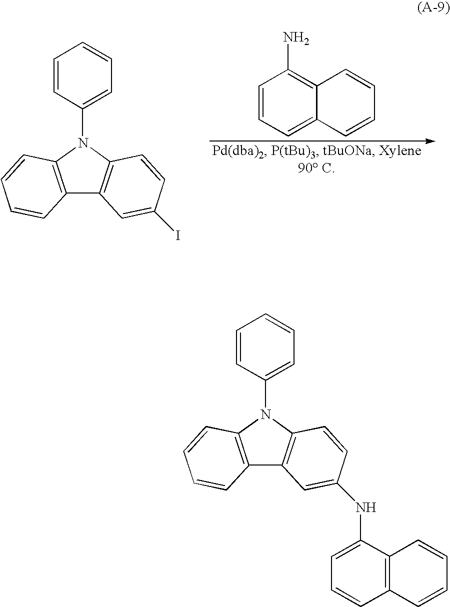

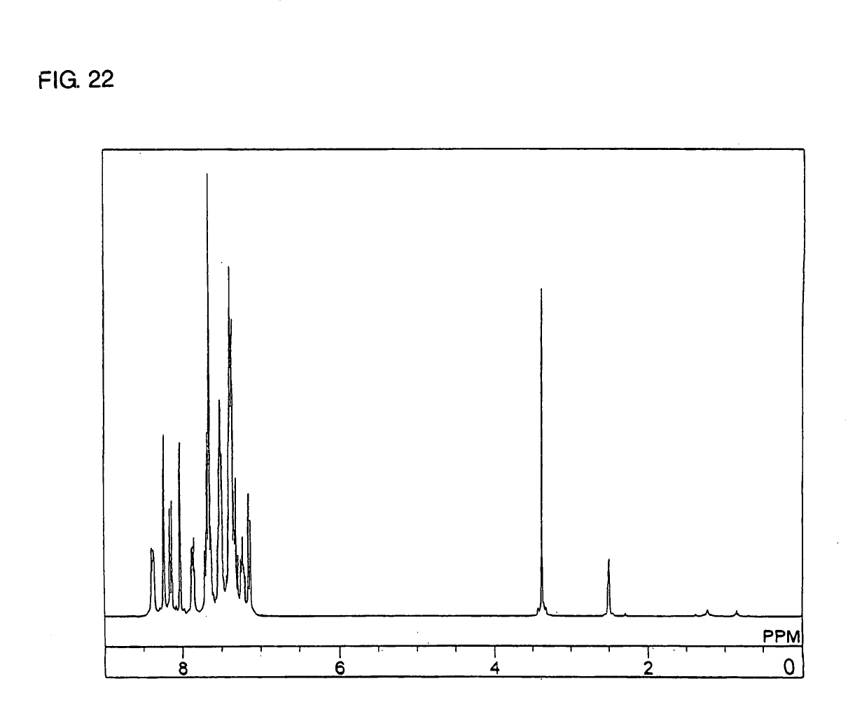

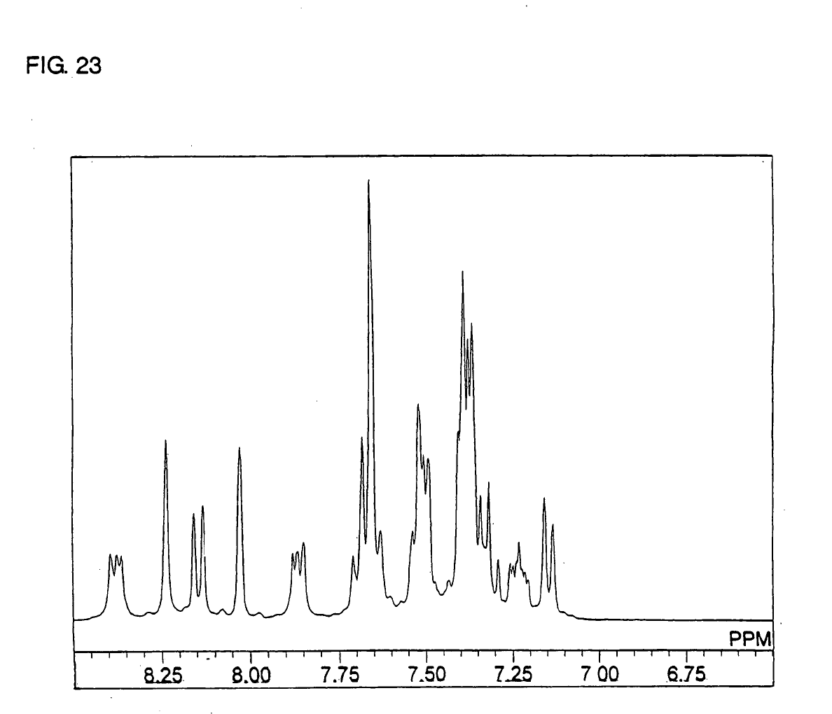

Under nitrogen, 12 ml of dehydrated xylene was added to a mixture of 3.7 g (10 mmol) of 3-iodo-9-phenylcarbazole, 1.6 g (5 mmol) of 1-aminonaphtalene, 60 mg (0.1 mmol) of bis(dibenzylidencacetone)palladium(0), 0.2 mL of a hexane solution with 49 wt % of tri-tert-butylphosphine, and 3.0 g (30 mmol) of sodium-tert-butoxide. This mixture was stirred while heating under nitrogen atmosphere at 90° C. for 7 hours. After the termination of the reaction, about 200 ml of heated toluene was added to the suspension and this suspension was filtered through florisil, alumina and Celite®. The thus obtained filtrate was concentrated and the concentrated solution was purified by using silica gel column chromatography (toluene:hexane=1:1). This was concentrated and the obtained concentrated solution was recrystallized with hexane-ethyl acetate. 1.5 g (the yield: 79%) of 3-[N-(1-naphtyl)amino]-9-phenylcarbazole which was cream-colored powder, was obtained. NMR data are shown below. ¹H-NMR (300 MHz, DMSO-d): δ=7.13-7.71 (m, 15H), 7.85-7.88 (m, 1H), 8.03 (s, 1H), 8.15 (d, J=7.8, 1H), 8.24 (s, 1H), 8.36-8.39 (m, 1H). FIG. 22 shows a chart of ¹H-NMR, and FIG. 23 shows an enlarged view of the portion of 6.50 to 8.50 ppm in FIG. 22.

### [Step 2]

Next, a synthesis method of 3-[N-(1-naphtyl)-N-(9-phenylcarbazole-3-yl)amino]-9-phenylcarbazole (PCzPCN1) is described. A synthesis scheme of PCzPCN1 is represented by (A-10).

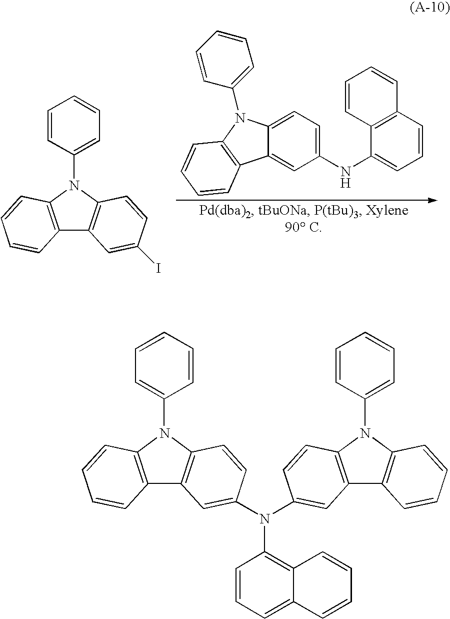

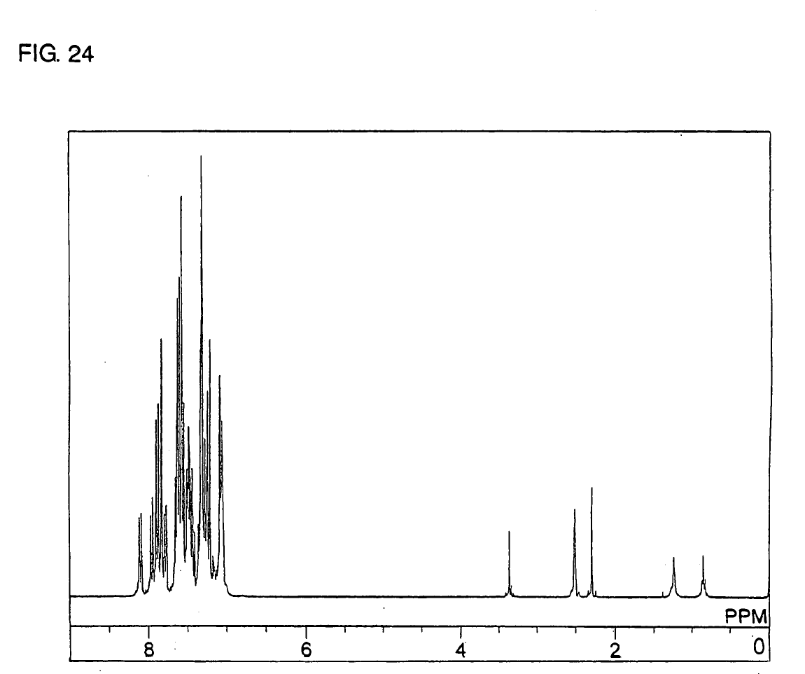

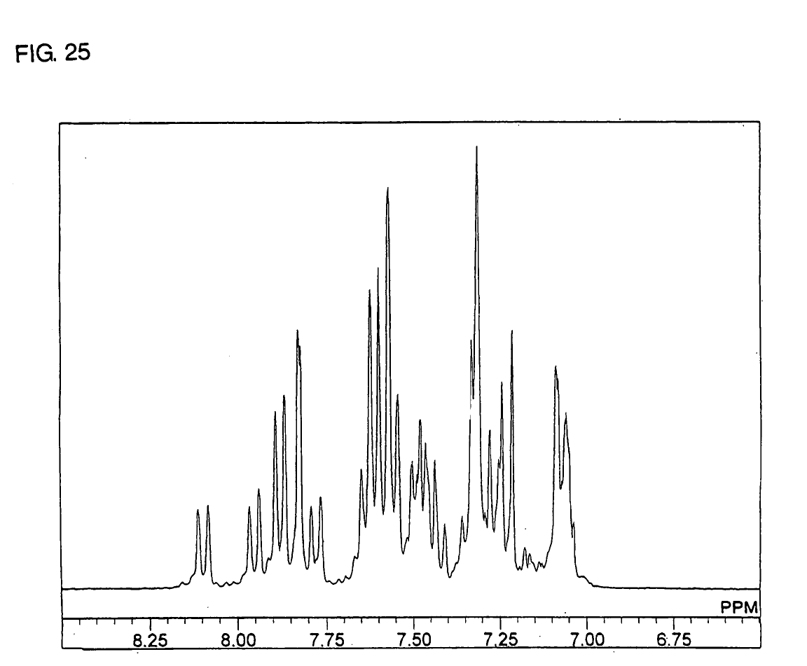

Under nitrogen, 7 ml of dehydrated xylene was added to a mixture of 1.8 g (5 mmol) of 3-iodo-9-phenylcarbazole, 2.5 g (6.6 mmol) of PCN, 30 mg (0.05 mmol) of bis(dibenzylideneacetone)palladium(0), 0.2 mL (0.5 mmol) of a hexane solution with 49 wt % of tri-tert-butylphosphine, and 700 mg (7 mmol) of sodium-tert-butoxide. This mixture was stirred while heating under nitrogen atmosphere at 90° C. for 4.5 hours. After the termination of the reaction, about 500 ml of heated toluene was added to the suspension and this suspension was filtered through florisil, alumina and Celite®. The thus obtained filtrate was concentrated and the concentrated solution was purified by using silica gel column chromatography (toluene:hexane=1:1). This was concentrated and the obtained concentrated solution was recrystallized with hexane-ethyl acetate. 2.1 g (the yield: 62%) of PCzPCN1 which was yellow powder, was obtained. NMR data are shown below. ¹H-NMR (300 MHz, DMSO-d): δ 7.04-7.65 (m, 24H), 7.78 (d, J=8.4, 1H), 7.82 (d, J=2.1, 2H), 7.88 (d, J=7.8, 2H), 7.95 (d, J=8.4, 1H), 8.10 (d, J=9.0, 1H). FIG. 24 shows a chart of ¹H-NMR, and FIG. 25 shows an enlarged view of the portion of 6.50 to 8.50 ppm in FIG. 24.

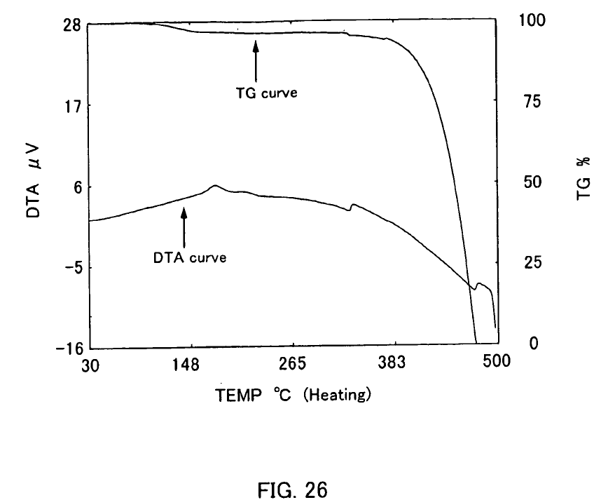

The thermogravimetry-differential thermal analysis (TG-DTA) of the thus obtained PCzPCN1 was performed in the same manner as Examples 1 and 2. The results are shown in FIG. 26. In FIG. 26, the vertical axis on the left side indicates the heat quantity (μV) and the vertical axis on the right side indicates the gravity (%; the gravity at the start of measurement is expressed as 100%). Furthermore, the lower horizontal axis represents a temperature (° C.). By utilizing a thermo-gravimetric/differential thermal analyzer (TG/DTA 320, Seiko Instruments Inc.), thermophysical properties were measured at the temperature rising velocity of 10° C./min under nitrogen atmosphere. As a result, based on the gravity-temperature relation (thermogravimetric measurement), the temperature at which the gravity becomes 95% or less of the gravity at the start of the measurement, was 400° C. under normal pressure.

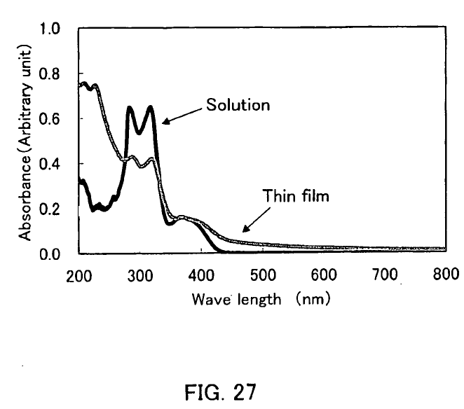

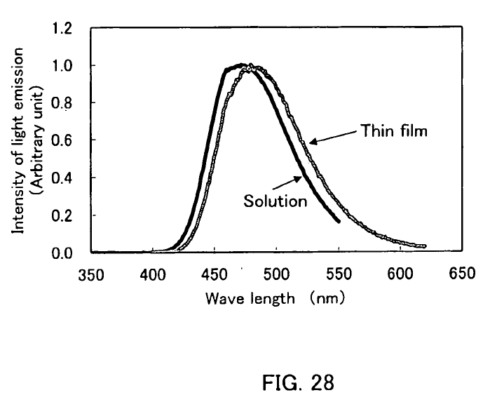

Absorption spectra of the toluene solution of PCzPCN1 and a thin film of PCzPCN1 are shown in FIG. 27. An ultraviolet-visible spectrophotometer (manufactured by JASCO Corporation, V-550) was used for the measurement. The solution was put in a quartz cell, and the thin film was deposited on a quartz substrate as samples, and absorption spectra of them, from which absorption spectrum of quartz was taken, are shown in FIG. 27. In FIG. 27, the horizontal axis indicates wavelength (nm) and the vertical axis indicates absorbance (arbitrary unit). The largest absorption wavelength was 314 nm in the case of the toluene solution, and 320 nm in the case of the thin film. Emission spectra of the toluene solution of PCzPCN1 and the thin film of PCzPCN1 are shown in FIG. 28. In FIG. 28, the horizontal axis indicates wavelength (nm) and the vertical axis indicates emission intensity (arbitrary unit). The highest emission wavelength was 475 nm (excitation wavelength 320 nm) in the case of the toluene solution, and 485 nm (excitation wavelength 320 nm) in the case of the thin film.

Further, the HOMO level and LUMO level of PCzPCN1 in a state of a thin film were measured. A value of the HOMO level was obtained by converting a value of ionization potential measured by a photoelectron spectrometer (manufactured by Riken Keiki Co., Ltd., AC-2) into a negative value. A value of the LUMO level was obtained by using an edge of the absorption spectrum of the thin film in FIG. 27 as an energy gap and adding the value of the absorption edge to the value of the HOMO level. As a result, the HOMO level and the LUMO level were −5.15 eV and −2.82 eV, respectively.

In addition, an oxidation reaction characteristic of the PCzPCN1 was measured by a cyclic voltammetry (CV) measurement. Further, an electrochemical analyzer (ALS model 600A, BAS Inc.) was used for the measurement.

As for a solution used in the CV measurement, dehydrated dimethylformamide (DMF) was used as a solvent. Tetraperchlorate-n-butylammonium (n-BU₄NClO₄), which was a supporting electrolyte, was dissolved in the solvent such that the concentration of the tetraperchlorate-n-butylammonium was 100 mmol/L. Also, the PCzPCN1, which was an object to be measured, was dissolved such that the concentration thereof was set to be 1 mmol/L Further, a platinum electrode (a PTE platinum electrode, BAS Inc.) was used as a work electrode. A platinum electrode (a VC-3 Pt counter electrode (5 cm), BAS Inc.) was used as an auxiliary electrode. An Ag/Ag⁺ electrode (an RE 5 nonaqueous reference electrode, BAS Inc.) was used as a reference electrode.

The oxidation reaction characteristic was measured as follows. After a potential of the work electrode with respect to the reference electrode was changed to 0.50 V from −0.20 V, a scan for changing the potential to −0.20 V from 0.50 V was set as one cycle, and 100 cycle measurements were carried out. Further, the scanning speed of the CV measurement was set to be 0.1 V/s.

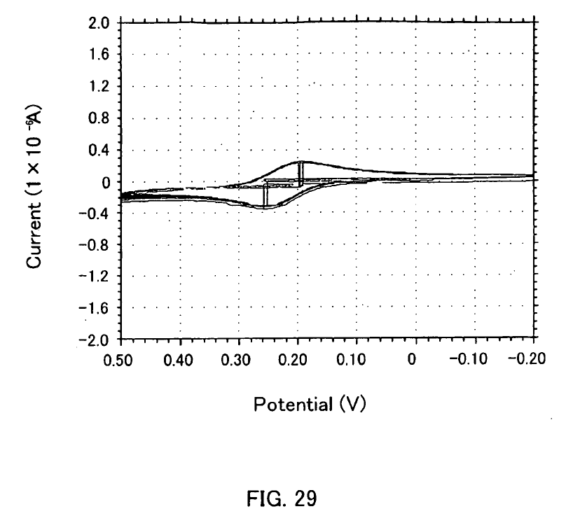

Results of measuring the oxidation reaction characteristic of the PCzPCN1 are shown in FIG. 29. In FIG. 29, the horizontal axis represents a potential (V) of the work electrode with respect to the reference electrode, while the vertical axis represents current flowing between the work electrode and the auxiliary electrode (1×10⁻⁶ A). According to FIG. 29, it was known that the oxidation potential was 0.25 V (vs; Ag/Ag⁺ electrode). Although the scan was repeated 100 times, the peak position and the peak intensity of a CV curve were hardly changed in the oxidation reaction. Thus, it was known that the carbazole derivative of the present invention, was absolutely stable for the oxidation reaction.

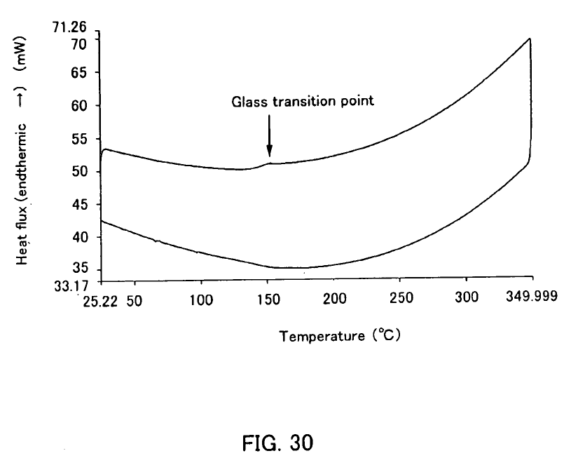

The glass transition temperature of the obtained compound PCzPCN1 was examined with a differential scanning calorimetry (Pyris 1 DSC manufactured by Perkin Elmer Co., Ltd.) Measurement results by DSC are shown in FIG. 30. According to the measurement results, it can be found that the glass transition temperature of the obtained compound was 142° C. As just described, the obtained compound exhibits a glass transition temperature of as high as 142° C., and has excellent heat resistance. In addition, the crystallization peak of the obtained compound is not shown in FIG. 30, and thus, it can be found that the obtained compound is difficult to be crystallized.

### EXAMPLE 4

Example 4 will describe another synthesis method of 3-[N-(9-phenyl carbazole-3-yl)-N-phenylamino]-9-phenylcarbazole (PCzPCA1), which is a different method from that of Example 1. A synthesis scheme of PCzPCA1 is shown by (D-1).

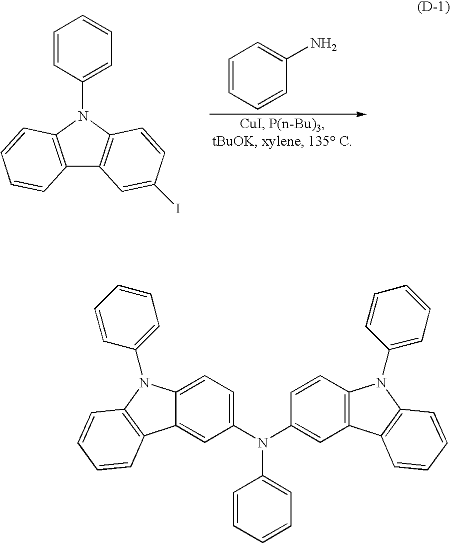

1.60 mg (4.33 mmol) of 3-iodo-9-phenylcarbazole, 19.0 mg (0.1 mmol) of copper iodide(I), 1.10 g (10 mmol) of tert-butoxy potassium, 1.0 mL of tri-n-butylphosphine (0.2 mol/L dehydrogenated hexane solution) were put in a 200-mL three-neck flask, and the atmosphere of the flask was substituted by nitrogen, 10 mL of xylene, 0.2 mL of aniline (2.1 mmol, 195.6 mg) were added thereto, and refluxed at 135° C. for 6 hours. The reaction solution was cooled at a room temperature, and 100 mL of toluene was added thereto, then the mixture was filtered through florisil, and Celite®. The obtained filtrate was washed with water twice and the water phase was extracted with toluene twice, the extracted solution and an organic phase which was washed with water were mixed and washed with saturated sodium chloride solution, and dried with magnesium sulfate. The solution was filtrated naturally, and a compound obtained by concentrating the filtrate was subjected to silica gel chromatography (a mixture solution of toluene and hexane) to obtain an objective substance. 140 mg (the yield: 21%) of a light-yellow solid was obtained.

By employing the synthesis method shown in Example 4, a carbazole derivative of the present invention can be obtained by one step of reaction.

### EXAMPLE 5

Example 5 will describe another synthesis method of 3-[N-(1-naphtyl)-N-(9-phenylcarbazole-3-yl)amino]-9-phenylcarbazole (PCzPCN1) represented by the structural formula (17), which is a different method from that of Example 3. A synthesis scheme of PCzPCN1 is shown by (D-2).

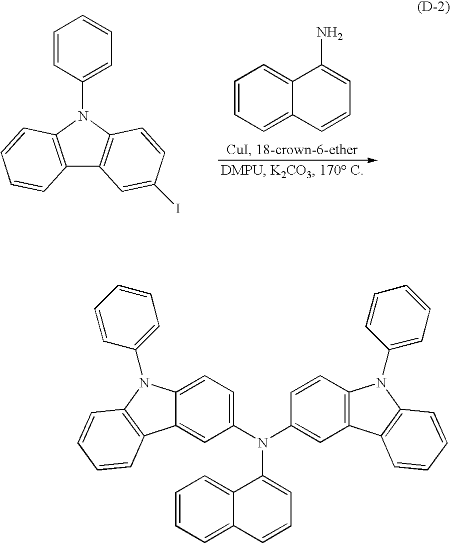

3.69 g (0.01 mol) of 3-iodo-9-phenylcarbazole, 716 mg (5 mmol) of 1-naphtylamine, 385 mg (2 mmol) of copper iodide, 2.74 g (0.02 mol) of potassium carbonate, and 771 mg (0.02 mol) of 18-crown 6-ether were put in a 200-mL three-neck flask, and the atmosphere of the flask was substituted by nitrogen, 8 mL of DMPU was added thereto, and stirred at 170° C. for 24 hours. The reaction solution was cooled at a room temperature, washed with water twice and water phase Was extracted with toluene twice, the extracted solution and organic phase, which had been washed in advance, were mixed and washed with saturated sodium chloride solution, and dried with magnesium sulfate. The solution was filtrated naturally, and a compound obtained by concentrating the filtrate was purified with silica gel chromatography (hexane:toluene=7:3) to obtain an objective substance, i.e., 1.52 g (the yield: 48%) of a light-yellow solid.

By employing the synthesis method shown in Example 5, a carbazole derivative of the present invention can be obtained by one step of reaction.

### EXAMPLE 6

A synthesis method of 3-{N-[9-(4-biphenylyl)carbazole-3-yl]-N-phenylamino}-9-(4-biphenylyl)carbazole (BCzBCA1), which is represented by the structural formula (70), will be described as one example of a carbazole derivative of the present invention.

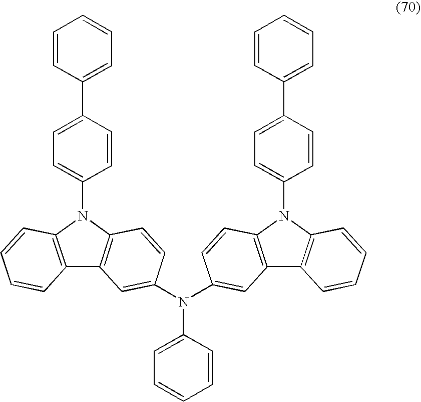

[Step 1] A synthesis method of 9-(4-biphenylyl)carbazole is described. A synthesis scheme of 9-(4-biphenylyl)carbazole is represented by (B-1).

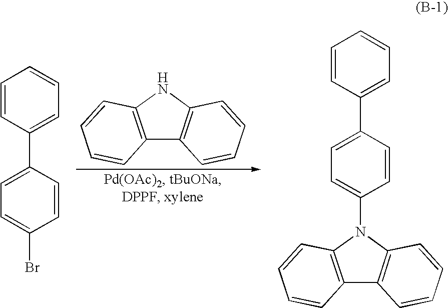

12 g (50 mmol) of 4-bromobiphenyl, 8.4 g (50 mmol) of carbazole, 230 mg (1 mmol) of palladium acetate, 1.8 g (3.0 mmol) of 1,1-bis(diphenylphosphino)ferrocene, and 13 g (180 mmol) of sodium-tert-butoxide were added in a three-neck flask and the atmosphere of the flask was substituted by nitrogen, and then, 80 mL of dehydrogenated xylene was added, and deaerated. Under nitrogen atmosphere, it was stirred at 120° C. for 7.5 hours. After the termination of the reaction, about 600 mL of heated toluene was added to this suspension, and filtrated twice through florisil, alumina and Celite®. The obtained filtrate was concentrated and hexane was added thereto, recrystallization was conducted. This was filtrated, and the residue was collected and dried to obtain 14 g (the yield: 87%) of 9-(4-biphenylyl) carbazole which was cream-colored powder.

[Step 2] A synthesis method of 3-bromo-9-(4-biphenylyl)carbazole is described. A synthesis scheme of 3-bromo-9-(4-biphenylyl)carbazole is shown by (B-2).

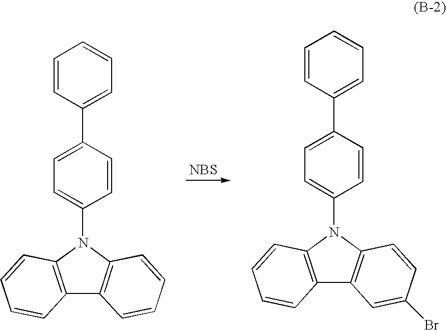

3.1 g (10 mmol) of 9-(4-biphenylyl)carbazole was dissolved in 100 mL of chloroform, and 1.8 g (10 mmol) of N-bromosuccinimide was added thereto slowly. This was stirred overnight (for about 24 hours), and then, washed with water. Magnesium sulfate was added to this to remove water and it was filtered. The filtrate was concentrated to be collected, and dried. 3.7 g (the yield: 95%) of 3-bromo-9-(4-biphenylyl)carbazole, which was beige powder, was obtained.

[Step 3] A synthesis method of 3-iodo-9-(4-biphenylyl)carbazole is described. A synthesis scheme of 3-iodo-9-(4-biphenylyl)carbazole is represented by (B-3).

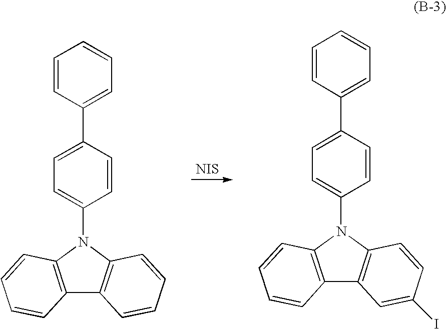

3.2 g (10 mmol) of 9-(4-biphenylyl) carbazole was dissolved in a mixture solution of 200 mL of glacial acetic acid, 200 mL of toluene and 50 mL of ethyl acetate. 2.3 g (10 mmol) of N-iodosuccinimide was added thereto slowly. This was stirred overnight (for about 24 hours), and then, washed with water, a sodium thiosulfate water solution, and saturated sodium chloride solution. Magnesium sulfate was added to this to remove water. This was concentrated and added with acetone and hexane, then recrystallized with ultra sonic wave. This was filtered to obtain a residue. The residue was collected and dried. 4.4 g (the yield: 98%) of 3-iodo-9-(4-biphenylyl)carbazole, which was beige powder, was obtained.

[Step 4A] synthesis method of N-[(4-biphenylyl)carbazole-3-yl]-N-phenylamine (BCA) is described. A synthesis scheme of BCA is represented by (B-4).

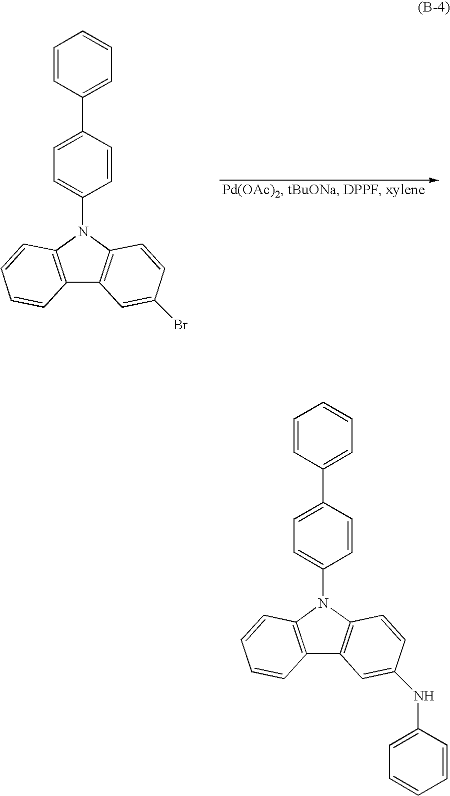

3.7 g (9.2 mmol) of 3-bromo-9-(4-biphenyl)carbazole, 63 mg (0.3 mmol) of palladium acetate, 330 mg (0.6 mmol) of 1,1-bis(diphenylphosphino) ferrocene, and 1.5 g (15 mmol) of sodium-tert-butoxide were added in a three-neck flask and the atmosphere of the flask was substituted by nitrogen, and then, 20 mL of dehydrogenated xylene was added, and deaerated. Then, 9.3 g (10 mmol) of aniline was added thereto. Under nitrogen atmosphere, it was stirred at 130° C. for 4 hours. After the termination of the reaction, about 300 mL of heated toluene was added to this suspension, and filtrated through florisil, alumina and Celite®. The obtained residue was concentrated and hexane was added thereto. Then, it was precipitated by ultra sonic wave. This was filtrated, and the filtrate was dried to obtain 3.5 g (the yield: 93%) of N-[(4-biphenylyl)carbazole-3-yl]-N-phenylamine (BCA) which was cream-colored powder.

[Step 5] A synthesis method of 3-{N-[9-(4-biphenyl yl)carbazole-3-yl]-N-phenylamino}-9-(4-biphenylyl)carbazole (BCzBCA1) is described. A synthesis scheme of BCzBCA1 is represented by (B-5).

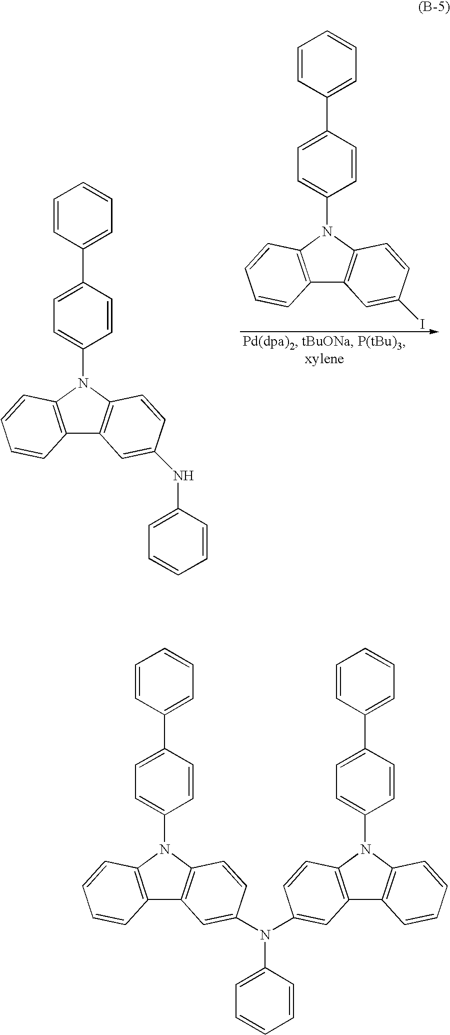

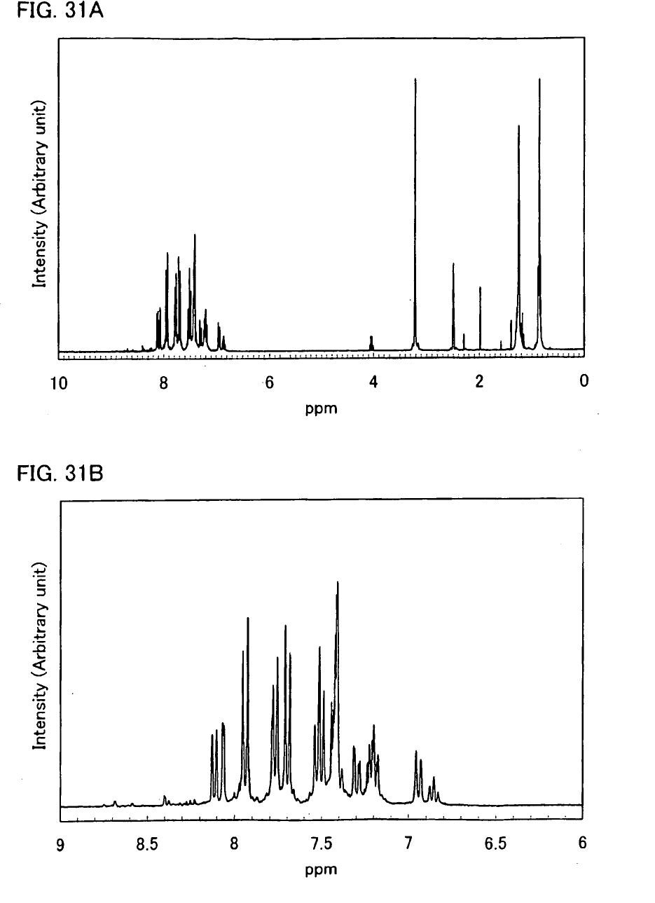

3.5 g (7.9 mmol) of 3-iodo-9-(4-biphenyl)carbazole, 3.3 g (8.0 mmol) of N-[(4-biphenyl)carbazole-3-yl]-N-phenylamine, 230 mg (0.4 mmol) of bis(dibenzylideneacetone)palladium (0) and 1.2 g (12 mmol) of sodium-tert-butoxide were added in a three-neck flask and the atmosphere of the flask was substituted by nitrogen, and then, 30 mL of dehydrogenated xylene was added, and deaerated. 1.4 mL (1.2 mmol) of a hexane solution with 10 wt % of tri-tert-butylphosphine was added thereto. Under nitrogen atmosphere, it was stirred at 110° C. for 3 hours. After the termination of the reaction, about 500 mL of heated toluene was added to this suspension, and filtrated through florisil, alumina and Celite®. The obtained filtrate was concentrated and was obtained using silica gel column chromatography (toluene:hexane=1:1). This was concentrated and hexane is added thereto. Then, it was precipitated by ultra sonic wave. 1.1 g (the yield: 19%) of 3-{N-[9-(4-biphenylyl)carbazole-3-yl]-N-phenylamino}-9-(4-biphenylyl)carbazole (BCzBCA1), which was cream-colored powder, was obtained. Data of ¹H-NMR was shown below. ¹H-NMR (300 MHz, DMSO-d): δ=6.86 (t, J=7.2, 1H), 6.94 (d, i=7.8, 2H), 7.18-7.24 (m, 4H), 7.30 (dd, J=8.9, 1.8, 2H), 7.41-7.54 (m, 12H), 7.70 (d, J=8.4, 4H), 7.77 (d, J=7.2, 4H), 7.94 (d, J=8.4, 4H), 8.06 (d, J=2.1, 2H), 8.12 (d, J=7.8, 2H). FIGS. 31A and 31B each show a chart of ¹H-NMR, and FIG. 31B shows an enlarged view of the portion of 6.0 to 9.0 ppm in FIG. 31A. Data of ¹³C-NMR was shown below. (75.5 MHz, DMSO-d): δ=109.6, 110.7, 117.4, 119.4, 119.7, 119.8, 120.5, 120.5, 122.4, 123.7, 125.0, 126.2, 126.5, 126.8, 127.5, 128.1, 128.8, 136.0, 136.9, 139.1, 139.1, 140.6, 140.8, 149.3. FIG. 32 shows a chart of ¹³C-NMR. FIG. 32B shows an enlarged view of the portion of 6.0 to 9.0 ppm in FIG. 32A.

The thermogravimetry-differential thermal analysis (TG-DTA) of the thus obtained BCzBCA1 was performed in the same manner as Examples 1 to 3. By utilizing a thermo-gravimetric/differential thermal analyzer (TG/DTA 320, Seiko Instruments Inc.), thermophysical properties were measured at the temperature rising velocity of 10° C./min under nitrogen atmosphere. As a result, based on the gravity-temperature relation (thermogravimetric measurement), the temperature at which the gravity becomes 95% or less of the gravity at the start of the measurement, was 425° C. under normal pressure.

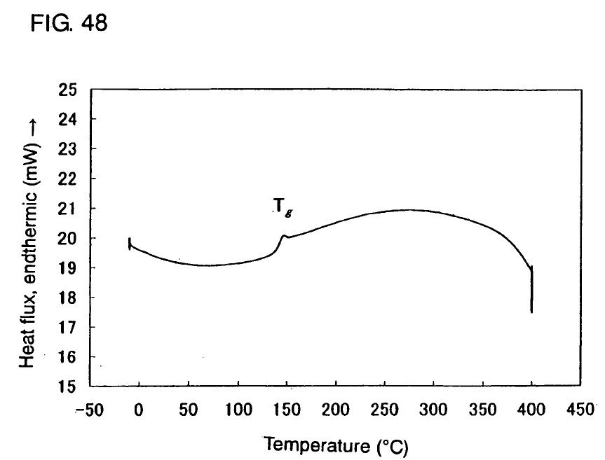

The glass transition temperature (T_{g}) was measured with a differential scanning calorimeter (Pyris 1 DSC manufactured by Perkin Elmer Co., Ltd.). After the sample was heated to 400° C. from −10° C. at 40° C./min, it was cooled at 40° C./min. After that, the temperature was risen up to 400° C. at 10° C./min, and thus, a DSC chart shown in FIG. 48 was obtained. According to this chart, it can be found that the glass transition temperature (Tg) of BCzBCA1 was 137° C. Therefore, it can be understood that BCzBCA1 had a high glass transition temperature. In this measurement, an absorption peak which shows a melting point was not observed.

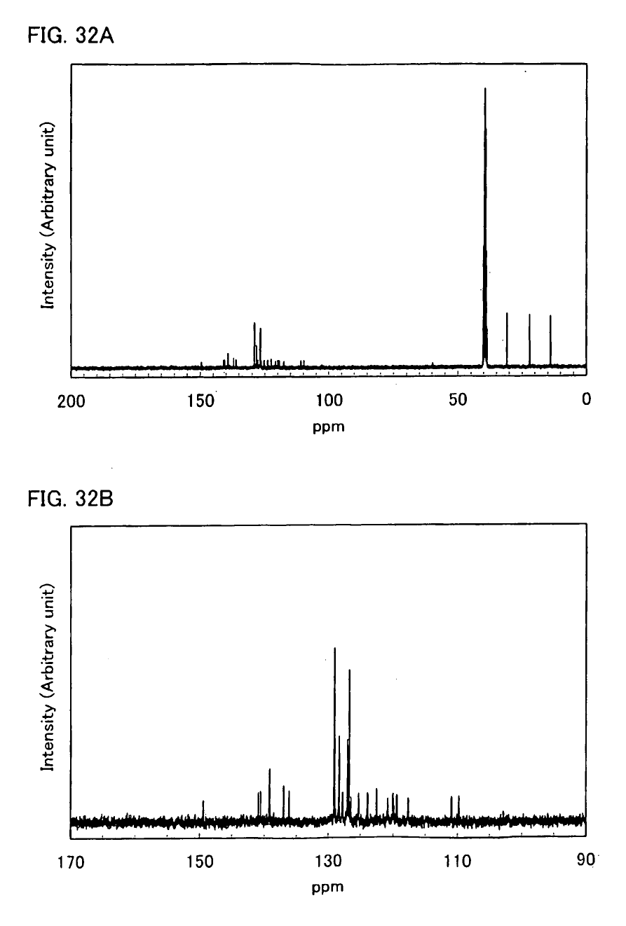

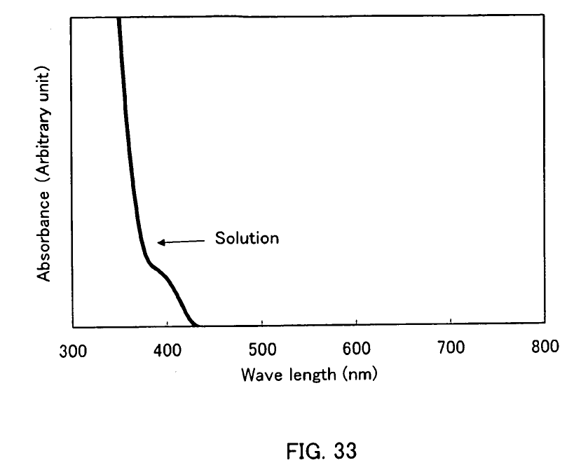

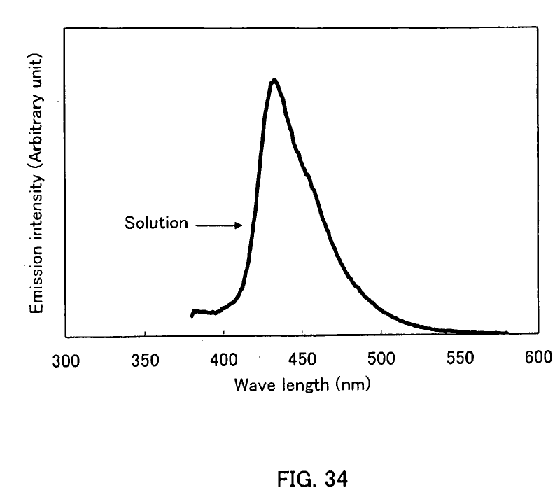

An absorption spectrum of the toluene solution of BCzBCA1 was shown in FIG. 33. An ultraviolet-visible spectrophotometer (manufactured by JASCO Corporation, V-550) was used for the measurement. The solution was put in a quartz cell as a sample, and the absorption spectrum, from which an absorption spectrum of quartz was taken, is shown in FIG. 33. In FIG. 33, the horizontal axis indicates wavelength (nm) and the vertical axis indicates absorbance (arbitrary unit). The largest absorption wavelength was 395 nm in the case of the toluene solution. Emission spectra of the toluene solution of BCzBCA1 is shown in FIG. 34. In FIG. 34, the horizontal axis indicates wavelength (nm) and the vertical axis indicates emission intensity (arbitrary unit). The highest emission wavelength was 434 nm (excitation wavelength 323 nm) in the case of the toluene solution.

### EXAMPLE 7

A synthesis method of 3,6-bis[N-(1-naphtyl)-N-(9-phenylcarbazole-3-yl)amino]-9-(4-biphenylyl)carbazole (BCzPCN2), which is represented by the structural formula (71), will be described as one example of a carbazole derivative of the present invention.

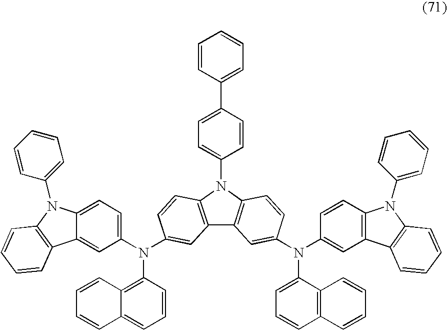

[Step 1] A synthesis method of 3,6-dibromo-9-(4-biphenylyl)carbazole is described. A synthesis scheme of 3,6-dibromo-9-(4-biphenylyl)carbazole is represented by (C-1).

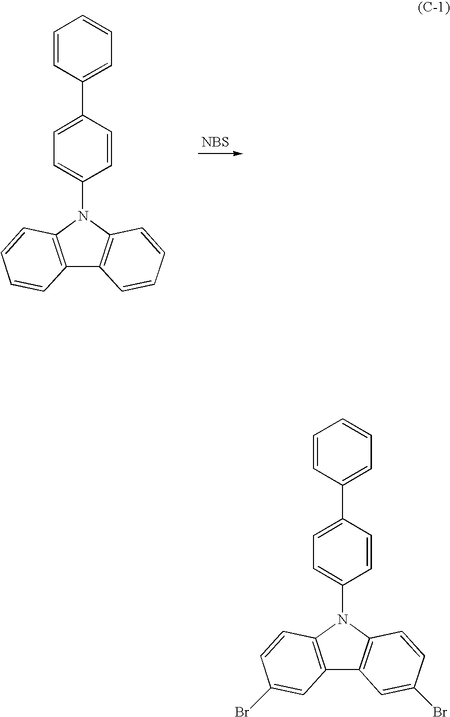

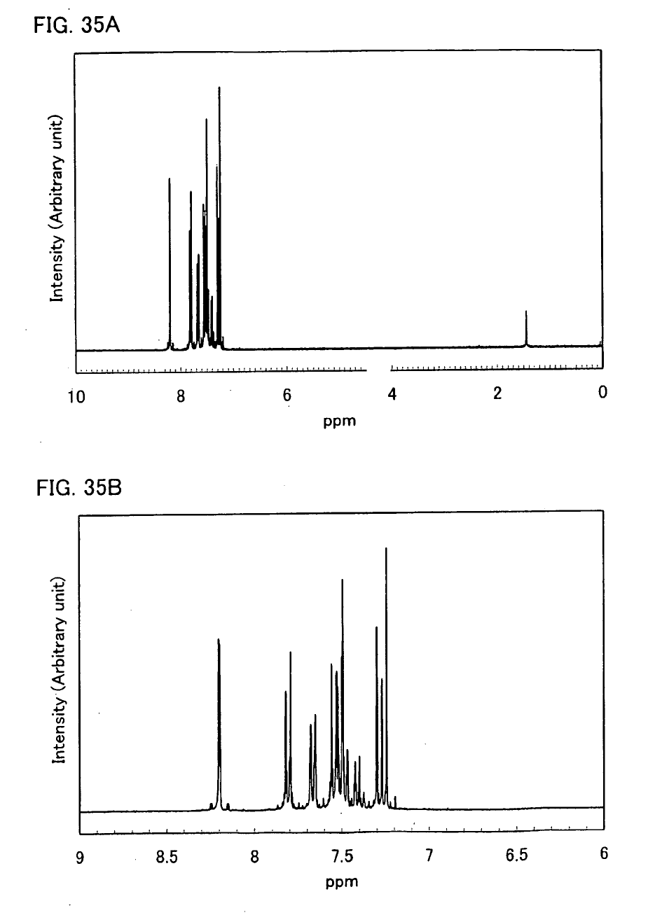

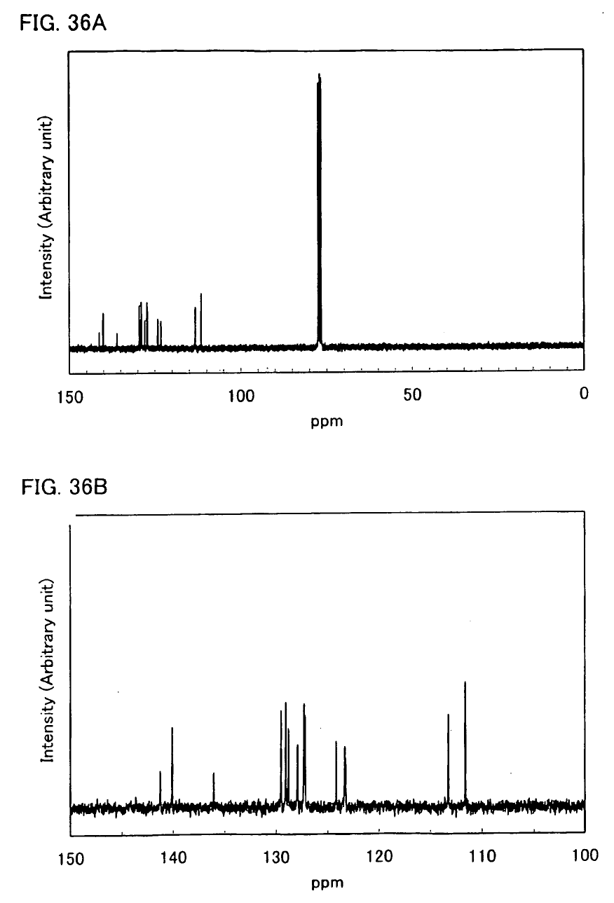

9.6 g (30 mmol) of 9-(4-biphenylyl)carbazole was dissolved in a mixture solution of glacial acetic acid 50 mL, toluene 250 mL and ethyl acetate 250 mL. 13 g (75 mmol) of N-bromosuccinimide was added thereto slowly. This was stirred for 5 days (for about 100 hours), and then, washed with water, a sodium thiosulfate water solution, and was neutralized by a sodium hydroxide solution, and then, washed with water again. Magnesium sulfate was added to this to remove water and filtered to obtain a filtrate. This was concentrated to be collected, and dried. 15 g of 3,6-dibromo-9-(4-biphenylyl)carbazole, which was beige powder, was obtained with yield of 100%. Data of NMR is shown below. ¹H-NMR (300 MHz, CDCl₃-d) δ=7.29 (d, J=8.7, 2H), 7.40 (t, J=7.5, 1H), 7.47-7.56 (m, 6H), 7.67 (d, J=7.5, 2H), 7.81 (d, J=8.4, 2H), 8.20 (d, J=2.1, 2H). FIG. 35 shows a chart of ¹H-NMR. FIG. 35B shows an enlarged view of the portion of 6.0 to 9.0 ppm in FIG. 35A. Data of ¹³C-NMR is shown below. ¹³C-NMR (75.5 MHz, CDCl₃-d); δ=111.6, 113.3, 123.3, 123.3, 124.2, 127.2, 127.3, 127.9, 128.8, 129.0, 129.5, 136.1, 140.1, 141.3. FIG. 36 shows a chart of ¹³C-NMR. FIG. 36B shows an enlarged view of the portion of 100 to 150 ppm in FIG. 36A.

[Step 2] A synthesis method of 3,6-bis[N-(1-naphtyl)-N-(9-phenylcarbazole-3-yl)amino]-9-(4-biphenylyl)carbazole (BCzPCN2) is described. A synthesis scheme of BCzPCN2 is represented by (C-2).

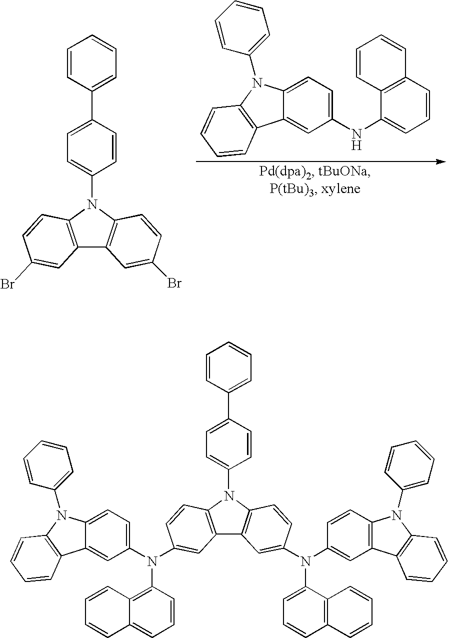

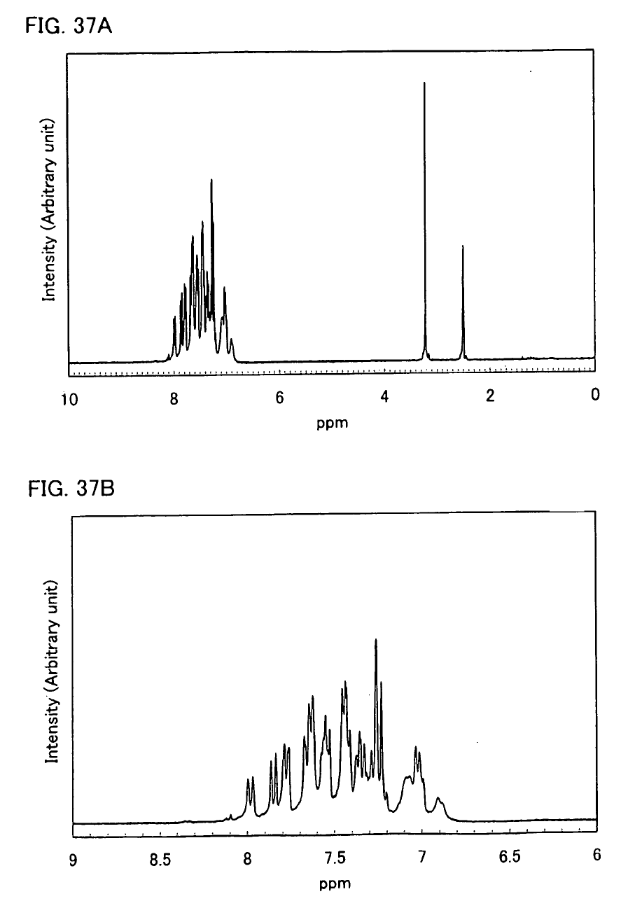

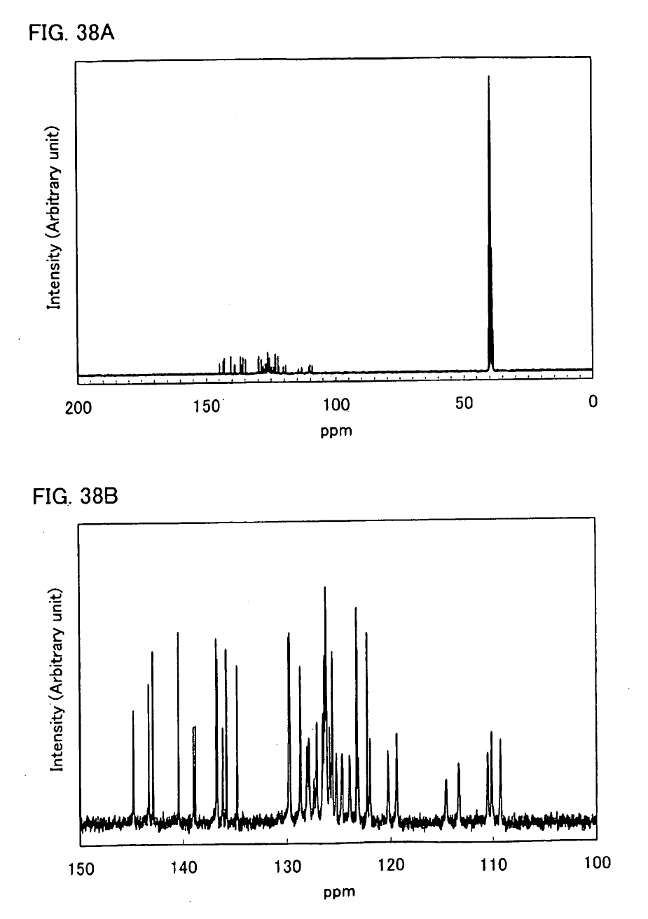

2.4 g (5.0 mmol) of 3,6-dibromo-9-(4-biphenylyl)carbazole, 3.8 g (10 mmol) of PCN, 580 mg (1.0 mmol) of bis(dibenzylideneacetone) palladium (0), 6.0 mL (3 mmol) of a hexane solution with 10 wt % of tri-tert-butylphosphine, and 3.0 g (30 mmol) of sodium-tert-butoxide were added and the atmosphere of the flask was substituted by nitrogen, and then, 10 mL of dehydrogenated xylene was added, and deaerated. This was heated to be stirred at 130° C. for 12 hours. After the termination of the reaction, about 550 mL of heated toluene was added to this suspension, and filtrated through florisil, alumina and Celite®. The obtained filtrate was concentrated and was obtained using silica gel column chromatography (toluene:hexane=2:1). This was concentrated and hexane was added thereto. Then, it was precipitated by ultra sonic wave. 2.7 g (the yield: 51%) of 3,6-bis[N-(1-naphtyl)-A-(9-phenylcarbazole-3-yl)amino]-9-(4-biphenylyl)carbazole (BCzPCN2), which was a lemon-colored powder, was obtained. Data of NMR is shown below. ¹H-NMR (300 MHz, DMSO-d): δ=6.88-7.67 (m, 45H), 7.76-7.79 (d, J=7.8, 4H), 7.84-7.86 (d, J=7.8, 2H), 7.97-7.99 (d, J=7.8, 2H). FIGS. 37A and 37B each show a chart of ¹H-NMR, and FIG. 37B shows an enlarged view of the portion of 6.0 to 9.0 ppm in FIG. 37A. Data of ¹³C-NMR is shown below. (75.5 MHz, DMSO-d): δ=109.3, 110.1, 110.5, 113.3, 113.3, 114.5, 114.6, 119.4, 120.2, 122.0, 122.2, 123.1, 123.2, 123.3, 124.0, 124.7, 125.2, 125.6, 125.9, 126.2, 126.4, 126.5, 127.1, 127.4, 127.9, 128.1, 128.7, 129.7, 129.8, 134.8, 135.8, 136.1, 136.7, 136.8, 138.8, 139.0, 140.4, 142.9, 143.3, 144.8. FIGS. 38A and 38B each show a chart of ¹³C-NMR. FIG. 38B shows an enlarged view of the portion of 100 to 150 ppm in FIG. 38A.

The thermogravimetry-differential thermal analysis (TG-DTA) of the thus obtained BCzPCN2 was performed in the same manner as Examples 1 to 4. By utilizing a thermo-gravimetric/differential thermal analyzer (TG/DTA 320, Seiko Instruments Inc.), thermophysical properties were measured at the temperature rising velocity of 10° C./min under nitrogen atmosphere. As a result, based on the gravity-temperature relation (thermogravimetric measurement), the temperature at which the gravity becomes 95% or less of the gravity at the start of the measurement, was 500° C. or more under normal pressure.

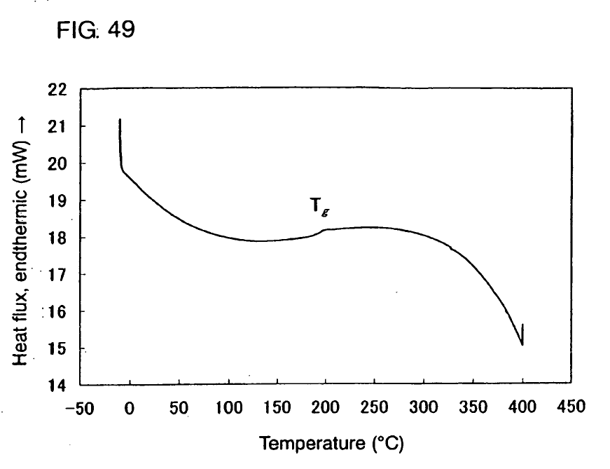

The glass transition temperature (T_{g}) was measured with a differential scanning calorimeter (Pyris 1 DSC manufactured by Perkin Elmer Co., Ltd.). After the sample was heated to 400° C. from −10° C. at 40° C./min, it was cooled to −10° C. at 40° C./min. After that, the temperature was risen up to 400° C. at 10° C./min, and thus, a DSC chart shown in FIG. 49 as obtained. According to this chart, it can be found that the glass transition temperature (Tg) of BCzPCN2 was 185° C. Therefore, it can be understood that BCzPCN2 had a high glass transition temperature. In this measurement, an absorption peak which shows a melting point was not observed.

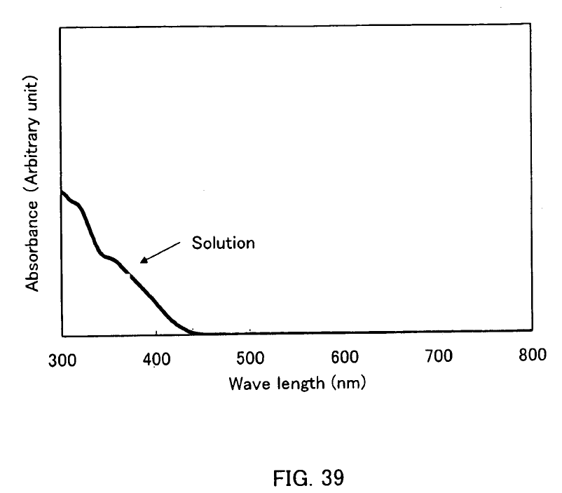

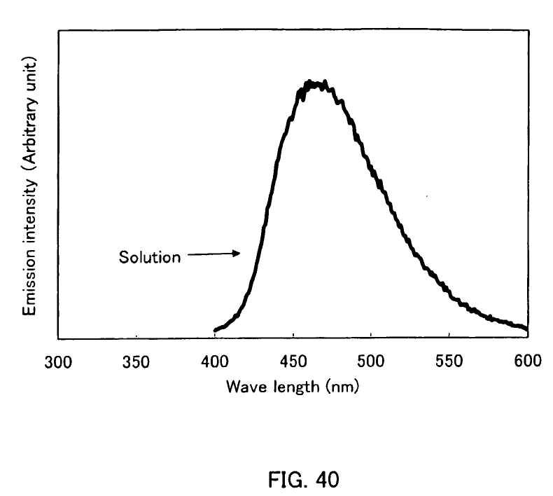

An absorption spectrum of the toluene solution of BCzPCN2 is shown in FIG. 39. An ultraviolet-visible spectrophotometer (manufactured by JASCO Corporation, V-550) was used for the measurement. The solution was put in a quartz cell as a sample, and the absorption spectrum, from which absorption spectrum of quartz was taken, is shown in FIG. 39. In FIG. 39, the horizontal axis indicates wavelength (nm) and the vertical axis indicates absorbance (arbitrary unit). The largest absorption wavelength was 370 nm in the case of the toluene solution. The emission spectrum of the toluene solution of BCzPCN2 is shown in FIG. 40. In FIG. 40, the horizontal axis indicates wavelength (nm) and the vertical axis indicates emission intensity (arbitrary unit). The highest emission wavelength was 465 nm (excitation wavelength 320 nm) in the case of the toluene solution.

### EXAMPLE 8

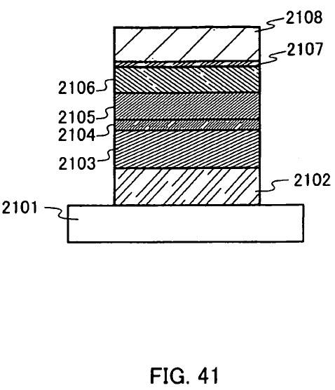

Example 8 describes a light-emitting element using a carbazole derivative of the present invention with reference to FIG. 41.

Indium in oxide including silicon oxide was formed by sputtering over a glass substrate 2101 first to form a first electrode 2102. The thickness was 110 nm and the area of the electrode was 2 mm×2 mm.

The substrate provided with the first electrode was fixed on a substrate holder which was provided in a vacuum evaporation apparatus, in such a way that the surface provided with the first electrode faces downwardly. After that, the air inside the vacuum evaporation apparatus was evacuated to about 10⁻⁴ Pa. Then, a 50-nm thick film of 3-[N-(9-phenylcarbazole-3-yl)-N-phenylamino]-9-phenylcarbazole (PCzPCA1) represented by the structural formula (12), was formed by an evaporation method using resistant heating, thereby forming a hole injecting layer 2103 on the first electrode 2102.

A 10-nm thick NPB film was formed over the hole injecting layer 2103 to form a hole transporting layer 2104 by an evaporation method using resistant heating.

Further, Alq and coumarin 6 were deposited by co-evaporation, and a 40-nm-thick light-emitting layer 2105 was formed over the hole transporting layer 2104. Herein, the weight ratio of Alq and coumarin 6 (Alq:coumarin 6) was adjusted to 1:0.01.

Thereafter, a 10-nm-thick Alq layer was formed over the light-emitting layer 2105 by an evaporation method using resistant heating, to form an electron transporting layer 2106.

Further, a 1-nm-thick lithium fluoride film was formed over the electron transporting layer 2106 to form an electron injecting layer 2107.

Lastly, a 200-nm-thick aluminum film was formed over the electron injecting layer 2107 by an evaporation method using resistant heating, to form a second electrode 2108. In this manner, the light-emitting element of Example 8 was manufactured.

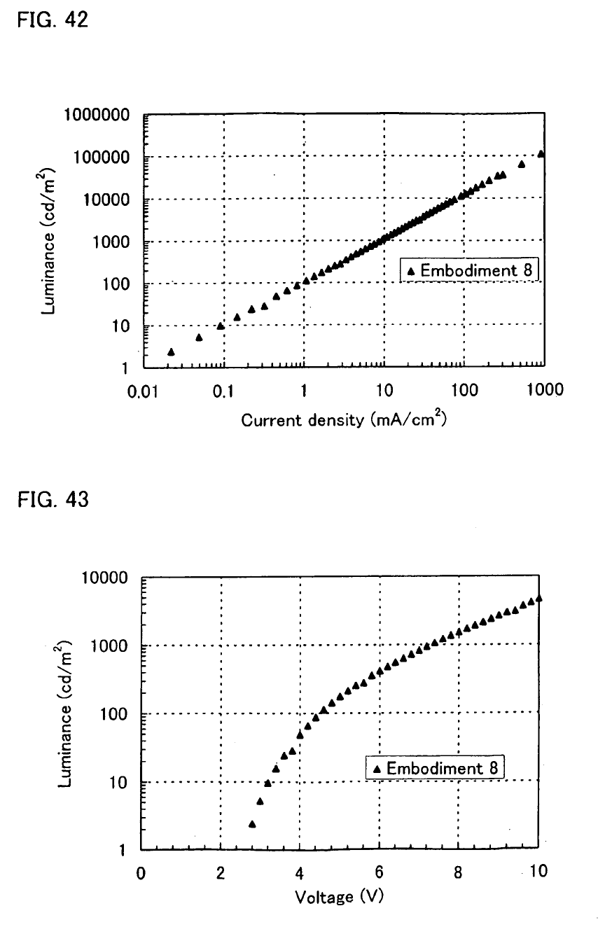

FIG. 42 shows current density-luminance characteristics of the light-emitting element of Example 8. FIG. 43 shows voltage-luminance characteristics. In the light-emitting element of Example 8, a voltage of 7.2 V was applied, and green light emission of CIE chromaticity coordinate (x, y)=(0.32, 0.61), which was derived from coumarin 6, was obtained in 940 cd/m².

As described above, a light-emitting element having an excellent property can be obtained by using a derivative carbazole of the present invention for a hole injecting layer.

### EXAMPLE 9

Example 9 describes a light-emitting element using a carbazole derivative of the present invention with reference to FIG. 41.

Indium tin oxide including silicon oxide was formed by sputtering over a glass substrate 2101 first, and a first electrode 2102 was formed. The thickness was 110 nm and the area of the electrode was 2 mm×2 mm.

A substrate provided with the first electrode was fixed on a substrate holder which was provided in a vacuum evaporation apparatus, in such a way that the surface provided with the first electrode faces downwardly. After that, the air inside the vacuum evaporation apparatus was evacuated to about 10⁻⁴ Pa. Then, a 50-nm thick film of 3,6-bis[N-(9-phenylcarbazole-3-yl)-N-phenylamino]-9-phenylcarbazole (PCzPCA2) represented by the structural formula (38) was formed by an evaporation using resistant heating, thereby forming a hole injecting layer 2103 on the first electrode 2102.

In the same manner as Example 8, a hole transporting layer, a light-emitting layer, an electron transporting layer, an electron injecting layer, and a second electrode were formed to manufacture a light-emitting element of Example 9.

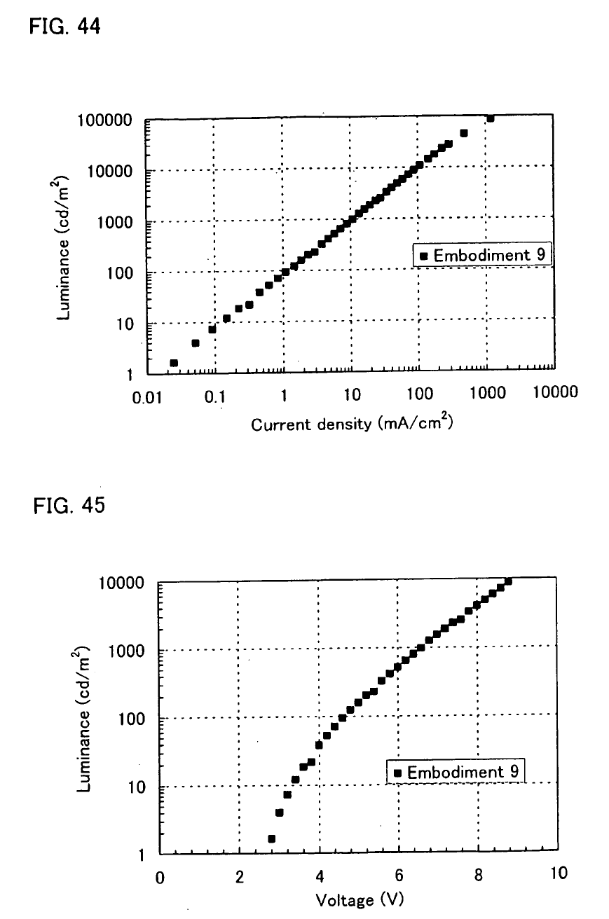

FIG. 44 shows current density-luminance characteristics of the light-emitting element of Example 9. FIG. 45 shows voltage-luminance characteristics. In the light-emitting element of Example 9, a voltage of 6.6 V was applied, and green light emission, which was derived from coumarin 6 of CIE chromaticity coordinate (x, y)=(0.32, 0.61) was obtained in 993 cd/m².

As described above, a light-emitting element having an excellent property can be obtained by using a derivative carbazole of the present invention for a hole injecting layer.

### EXAMPLE 10

Example 9 describes a light-emitting element using a carbazole derivative of the present invention with reference to FIG. 41.

Indium tin oxide including silicon oxide was formed by sputtering over a glass substrate 2101 first, and a first electrode 2102 was formed. The thickness was 110 nm and the area of the electrode was 2 mm×2 mm.

The substrate provided with the first electrode was fixed on a substrate holder which was provided in a vacuum evaporation apparatus, in such a way that the surface provided with the first electrode faces downwardly. After that, the air inside the vacuum evaporation apparatus was evacuated to about 10⁻⁴ Pa. Then, a 50-nm thick film of 3-[N-(1-naphtyl)-N-(9-phenylcarbazole-3-yl)amino]-9-phenylcarbazole (PCzPCN1) represented by the structural formula (17), was formed by an evaporation using resistant heating, thereby forming a hole injecting layer 2103 on the first electrode 2102.

In the same manner as Example 8, a hole transporting layer, a light-emitting layer, an electron transporting layer, an electron injecting layer, and a second electrode were formed to manufacture a light-emitting element of Example 10.

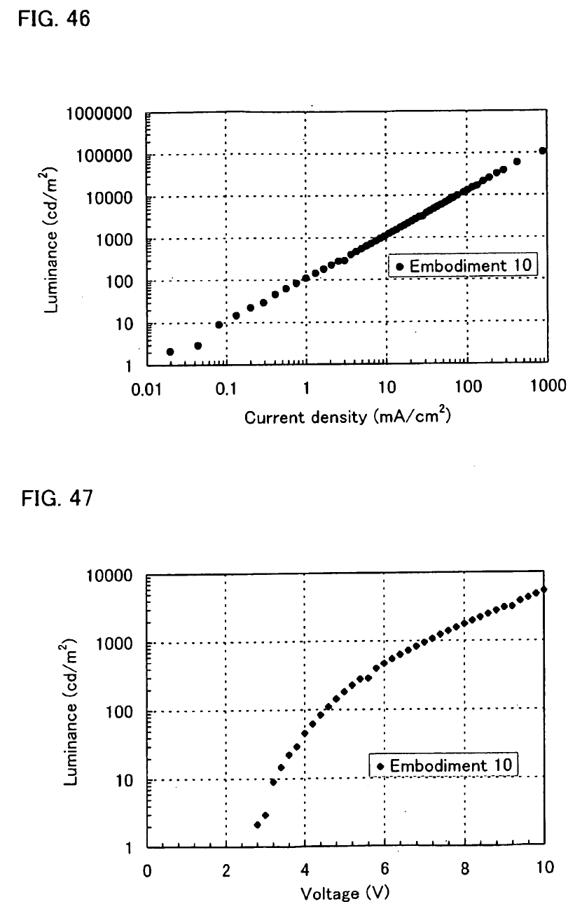

FIG. 46 shows current density-luminance characteristics of the light-emitting element of Example 10. FIG. 47 shows voltage-luminance characteristics. In the light-emitting element of Example 10, a voltage of 7.0 V was applied, and green light emission of CIE chromaticity coordinate (x, y)=(0.32, 0.61), which was derived from coumarin 6 was obtained in 946 cd/m².

As described above, a light-emitting element having an excellent property can be obtained by using a derivative carbazole of the present invention for a hole injecting layer.

This application is based on Japanese Patent Application serial No. 2004-381155 filed in Japan Patent Office on Dec. 28, 2004, and Japanese Patent Application serial No. 2005-085020 filed in Japan Patent Office on Mar. 23, 2005, the entire contents of which are hereby incorporated by reference.

1. A carbazole derivative represented by a general formula (1): 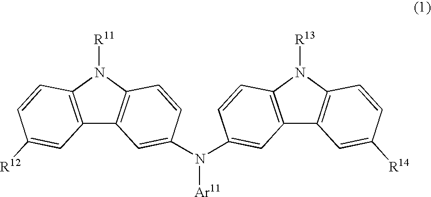
wherein at least one of R¹¹ and R¹³ represents hydrogen, an alkyl group having 1 to 6 carbon atoms, an aryl group having 6 to 25 carbon atoms, a heteroaryl group having 5 to 9 carbon atoms, an arylalkyl group, or an acyl group having 1 to 7 carbon atoms,
wherein Ar¹¹ represents an aryl group having 6 to 25 carbon atoms or a heteroaryl group having 5 to 9 carbon atoms,
wherein R¹² represents hydrogen, an alkyl group having 1 to 6 carbon atoms, or an aryl group having 6 to 12 carbon atoms,
wherein R¹⁴ represents hydrogen, an alkyl group having 1 to 6 carbon atoms, an aryl group having 6 to 12 carbon atoms, or a substituent shown by the general formula (2), 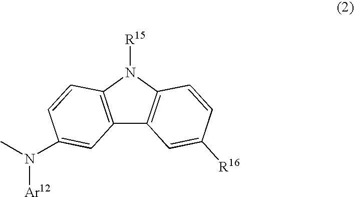
wherein R¹⁵ represents hydrogen, an alkyl group having 1 to 6 carbon atoms, an aryl group having 6 to 25 carbon atoms, a heteroaryl group having 5 to 9 carbon atoms, an aryl alkyl group, or an acyl group having 1 to 7 carbon atoms,
wherein Ar¹² represents an aryl group having 6 to 25-carbon atoms, or a heteroaryl group having 5 to 9 carbon atoms, and
wherein R¹⁶ represents hydrogen, an alkyl group having 1 to 6 carbon atoms, or an aryl group having 6 to 12 carbon atoms.

2. The carbazole derivative according to claim 1, wherein at least one of R¹¹ and R¹³ is an aryl group having 6 to 25 carbon atoms, or a heteroaryl group having 5 to 9 carbon atoms.

3. The carbazole derivative according to claim 1, wherein R¹¹ and R¹³ are the same.

4. The carbazole derivative according to claim 1, wherein at least one of R¹¹ and R¹³ is phenyl.

5. The carbazole derivative according to claim 1, wherein R¹² represents hydrogen, tert-butyl, phenyl, or biphenyl.

6. The carbazole derivative according to claim 1, wherein R¹⁴ represents hydrogen, tert-butyl, phenyl, or biphenyl.

7. The carbazole derivative according to claim 1, wherein R¹⁴ is a substituent represented by a general formula (2)

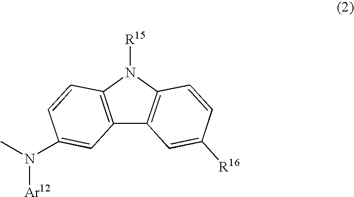

8. The carbazole derivative according to claim 7, wherein R¹⁵ represents an aryl group having 6 to 25 carbon atoms, or a hetero aryl group having 5 to 9 carbon atoms.

9. The carbazole derivative according to claim 7, wherein R¹⁶ represents hydrogen, tert-butyl, phenyl, or biphenyl.

10. A carbazole derivative represented by a general formula (3): 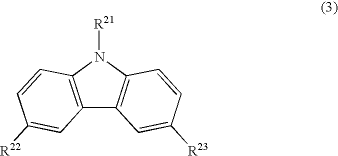
wherein R²¹ represents hydrogen, an alkyl group having 1 to 6 carbon atoms, an aryl group having 6 to 25 carbon atoms, a heteroaryl group having 5 to 9 carbon atoms, an arylalkyl group, or an acyl group having 1 to 7 carbon atoms,
wherein R²² represents hydrogen, an alkyl group having 1 to 6 carbon atoms, or an aryl group having 6 to 12 carbon atoms,
wherein R²³ represents a substituent shown by the general formula (4), 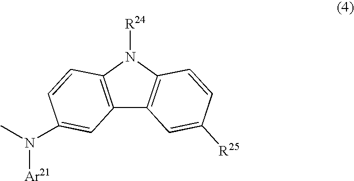
wherein R²⁴ represents hydrogen, an alkyl group having 1 to 6 carbon atoms, an aryl group having 6 to 25 carbon atoms, a heteroaryl group having 5 to 9 carbon atoms, an aryl alkyl group, or an acyl group having 1 to 7 carbon atoms,
wherein Ar²¹ represents an aryl group having 6 to 25 carbon atoms, or a heteroaryl group having 5 to 9 carbon atoms, and
wherein R²⁵ represents hydrogen, an alkyl group having 1 to 6 carbon atoms, or an aryl group having 6 to 12 carbon atoms.

11. The carbazole derivative according to claim 10, wherein R²² represents hydrogen, tert-butyl, phenyl, or biphenyl.

12. A carbazole derivative represented by a general formula (5): 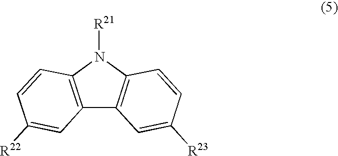
wherein R²¹ represents hydrogen, an alkyl group having 1 to 6 carbon atoms, an aryl group having 6 to 25 carbon atoms, a heteroaryl group having 5 to 9 carbon atoms, and an arylalkyl group, or an acyl group having 1 to 7 carbon atoms,
wherein R²² and R²³ represent a substituent represented by the general formula (6), 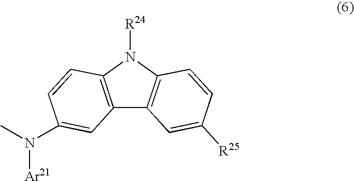
wherein R²⁴ represents hydrogen, an alkyl group having 1 to 6 carbon atoms, an aryl group having 6 to 25 carbon atoms, a heteroaryl group having 5 to 9 carbon atoms, an arylalkyl group, or an acyl group having 1 to 7 carbon atoms,
wherein Ar²¹ represents an aryl group having 6 to 25 carbon atoms, or a heteroaryl group having 5 to 9 carbon atoms, and
wherein R²⁵ represents hydrogen, an alkyl group having 1 to 6 carbon atoms, or a heteroaryl group having 6 to 12 carbon atoms.

13. The carbazole derivative according to claim 12, wherein R²⁵ represents hydrogen, tert-butyl, phenyl, or biphenyl.

14. The carbazole derivative according to claim 10, wherein R²⁴ represents an aryl group having 6 to 25 carbon atoms, or a hetero aryl group having 5 to 9 carbon atoms.

15. The carbazole derivative according to claim 10, wherein R²⁴ is phenyl.

16. The carbazole derivative according to claim 10, wherein R²¹ represents an aryl group having 6 to 25 carbon atoms, or a hetero aryl group having 5 to 9 carbon atoms.

17. The carbazole derivative according to claim 10, wherein R²¹ is phenyl.

18. A carbazole derivative represented by a general formula (7): 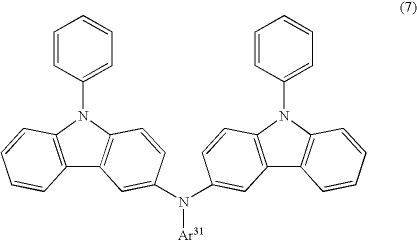
wherein Ar³¹ represents phenyl or naphtyl.

19. A carbazole derivative represented by a general formula (8) 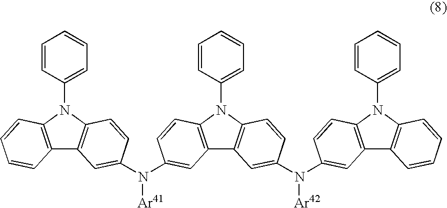
wherein Ar⁴¹ and Ar⁴² each are the same or different and represents one of phenyl and naphtyl.

20. A light-emitting element comprising:
an anode;
a cathode; and
a layer including a luminescent substance between the anode and the cathode, wherein the layer including a luminescent substance comprises the carbazole derivative according to claim 1.

21. The light-emitting element according to claim 20, wherein the carbazole derivative is included in a layer which is in contact with the anode.

22. The light-emitting element according to claim 20, wherein the carbazole derivative is included between the anode and a layer having a light-emitting layer which is included in the layer including a luminescent substance.

23. The light-emitting element according to claim 20, wherein the carbazole derivative is included in a light-emitting layer which is included in the layer including a luminescent substance.

24. A light-emitting device having the light-emitting element according to claim 20.

25. The carbazole derivative according to claim 12, wherein R²⁴ represents an aryl group having 6 to 25 carbon atoms, or a hetero aryl group having 5 to 9 carbon atoms.

26. The carbazole derivative according to claim 12, wherein R²⁴ is phenyl.

27. The carbazole derivative according to claim 12, wherein R²¹ represents an aryl group having 6 to 25 carbon atoms, or a hetero aryl group having 5 to 9 carbon atoms.

28. The carbazole derivative according to claim 12, wherein R²¹ is phenyl.

29. A light-emitting element comprising:
an anode;
a cathode; and
a layer including a luminescent substance between the anode and the cathode,
wherein the layer including a luminescent substance comprises the carbazole derivative according to claim 10.

30. The light-emitting element according to claim 29, wherein the carbazole derivative is included in a layer which is in contact with the anode.

31. The light-emitting element according to claim 29, wherein the carbazole derivative is included between the anode and a layer having a light-emitting layer which is included in the layer including a luminescent substance.

32. The light-emitting element according to claim 29, wherein the carbazole derivative is included in a light-emitting layer which is included in the layer including a luminescent substance.

33. A light-emitting device having the light-emitting element according to claim 29.

34. A light-emitting element comprising:
an anode;
a cathode; and
a layer including a luminescent substance between the anode and the cathode,
wherein the layer including a luminescent substance comprises the carbazole derivative according to claim 12.

35. The light-emitting element according to claim 34, wherein the carbazole derivative is included in a layer which is in contact with the anode.

36. The light-emitting element according to claim 34, wherein the carbazole derivative is included between the anode and a layer having a light-emitting layer which is included in the layer including a luminescent substance.

37. The light-emitting element according to claim 34, wherein the carbazole derivative is included in a light-emitting layer which is included in the layer including a luminescent substance.

38. A light-emitting device having the light-emitting element according to claim 34.

39. A light-emitting element comprising:
an anode;
a cathode; and
a layer including a luminescent substance between the anode and the cathode,
wherein the layer including a luminescent substance comprises the carbazole derivative according to claim 18.

40. The light-emitting element according to claim 39, wherein the carbazole derivative is included in a layer which is in contact with the anode.

41. The light-emitting element according to claim 39, wherein the carbazole derivative is included between the anode and a layer having a light-emitting layer which is included in the layer including a luminescent substance.

42. The light-emitting element according to claim 39, wherein the carbazole derivative is included in a light-emitting layer which is included in the layer including a luminescent substance.

43. A light-emitting device having the light-emitting element according to claim 39.

44. A light-emitting element comprising:
an anode;
a cathode; and
a layer including a luminescent substance between the anode and the cathode,
wherein the layer including a luminescent substance comprises the carbazole derivative according to claim 19.

45. The light-emitting element according to claim 44, wherein the carbazole derivative is included in a layer which is in contact with the anode.

46. The light-emitting element according to claim 44, wherein the carbazole derivative is included between the anode and a layer having a light-emitting layer which is included in the layer including a luminescent substance.

47. The light-emitting element according to claim 44, wherein the carbazole derivative is included in a light-emitting layer which is included in the layer including a luminescent substance.

48. A light-emitting device having the light-emitting element according to claim 44.
